(19) United States
(12) Patent Application Publication
(10) Pub. No.: US 2025/0177197 A1
(43) Pub. Date: Jun. 5, 2025

(54) PORTABLE TEMPERATURE REGULATION DEVICE

(71) Applicant: Shenzhen Lanhe Technologies Co., Ltd., Shenzhen (CN)

(72) Inventors: Weiping LI, Shenzhen (CN); Guang YANG, Shenzhen (CN); Kai LIU, Shenzhen (CN); Yanhui LI, Shenzhen (CN); Lechao LI, Shenzhen (CN); Guowei SONG, Shenzhen (CN); Jinya ZHEN, Shenzhen (CN); Yongjiong WANG, Shenzhen (CN); Junwei CHEN, Shenzhen (CN); You LAI, Shenzhen (CN); Yongsheng YANG, Shenzhen (CN)

(73) Assignee: Shenzhen Lanhe Technologies Co., Ltd., Shenzhen (CN)

(21) Appl. No.: 19/051,101

(22) Filed: Feb. 11, 2025

Related U.S. Application Data
(63) Continuation of application No. PCT/CN2023/112110, filed on Aug. 10, 2023.

(30) Foreign Application Priority Data
Aug. 12, 2022 (CN) 202222135020.2
Aug. 15, 2022 (CN) 202222148501.7
Aug. 22, 2022 (CN) 202222217831.7
Nov. 8, 2022 (CN) 202222986166.8
Dec. 1, 2022 (CN) 202223238968.7
Dec. 1, 2022 (CN) 202223238970.4
Dec. 1, 2022 (CN) 202223239202.0
Dec. 7, 2022 (CN) 202223287219.3
Dec. 19, 2022 (CN) 202223426607.5
Jan. 18, 2023 (CN) 202320180946.5
Feb. 6, 2023 (CN) 202320234673.8
Mar. 8, 2023 (CN) 202320543047.7
Mar. 30, 2023 (CN) 202320779420.9
Apr. 3, 2023 (CN) 202320761221.5

(51) Int. Cl. A61F 7/00

(52) U.S. Cl. CPC A61F 7/0085; A61F 7/007; A61F 2007/0011; A61F 2007/0024; A61F 2007/006; A61F 2007/0064; A61F 2007/0075

(57) ABSTRACT

A portable temperature regulation device defining a wear space, includes a main body, a protrusion portion, and a temperature conducting member at least partly arranged on the protrusion portion. The main body has a first side and a second side. The protrusion portion extends from the first side in a direction away from the second side. In use, the protrusion portion can extend toward a collar covering the back of the user or even to the collar such that the temperature conducting member arranged on the protrusion portion can contact the human back, so as to allow the temperature conducting member to transmit cold or heat to the human back, making it possible to realize temperature regulation even for the back area covered by the collar, meeting the need for cooling or hot compress massage on the human back and improving the user experience of the user.

### CROSS REFERENCE TO RELATED APPLICATIONS

The present application is a continuation application of PCT application No. PCT/CN2023/112110 filed on Aug. 10, 2023, which claims the priority of Chinese patent applications Nos. 202222135020.2 filed on Aug. 12, 2022, 202222148501.7 filed on Aug. 15, 2022, 202222217831.7 filed on Aug. 22, 2022, 202222986166.8 filed on Nov. 8, 2022, 202223238968.7 filed on Dec. 1, 2022, 202223239202.0 filed on Dec. 1, 2022, 202223238970.4 filed on Dec. 1, 2022, 202223287219.3 filed on Dec. 7, 2022, 202223426607.5 filed on Dec. 19, 2022, 202320180946.5 filed Jan. 18, 2023, 202320234673.8 filed on Feb. 6, 2023, 202320543047.7 filed on Mar. 8, 2023, 202320779420.9 filed on Mar. 30, 2023, and 202320761221.5 filed on Apr. 3, 2023. All of the above are hereby incorporated by reference in their entirety.

### TECHNICAL FIELD

The application relates to the technical field of temperature regulation, and more particularly to a portable temperature regulation device.

### BACKGROUND OF THE ART

In recent years, people are increasingly pursuing a more convenient life. In order to meet people's needs for convenient use of temperature regulation devices outdoors, various portable temperature regulation devices have appeared on the market, such as handheld fans and neck-mounted fans, which can be carried by people and can be used outdoors at any time.

However, as people's dependence on the use of portable temperature regulation devices and their functionality continue to increase, existing portable temperature regulation devices can no longer meet people's usage needs. Therefore, how to further improve the structure of portable temperature regulation devices has become an urgent problem to be solved.

### SUMMARY OF THE INVENTION

It is desired to provide an improved portable temperature regulation device.

A portable temperature regulation device defining a wear space, comprises a main body, a protrusion portion, and a temperature conducting member at least partly arranged on the protrusion portion. The main body has a first side and a second side opposite to the first side. The protrusion portion protrudes from the first side of the main body in a direction away from the second side.

The implementation of the technical solution of the application has at least the following beneficial effects: the protrusion portion extends toward a collar covering the human back or to even to inside of the collar such that the temperature conducting member arranged on the protrusion portion can contact the human back, so as to allow the temperature conducting member to transmit cold or heat to the human back, making it possible to realize temperature regulation even for the back area covered by the collar, meeting the need for adjusting cold or hot compress massage on the human back and improving the user experience of the user.

### BRIEF DESCRIPTION OF THE DRAWINGS

In order to more clearly illustrate the technical solutions in the embodiments of the application, the drawings that are necessarily used in the embodiments or the prior art description will be briefly introduced below. Obviously, the drawings described below are only some embodiments of the application. For ordinary technicians in this field, other drawings can be obtained based on these drawings without creative endeavor.

FIG. 1 is a perspective diagram showing a portable temperature regulation device according to a first embodiment of the application.

FIG. 2 is a perspective diagram, taken from a different angle, showing the portable temperature regulation device of FIG. 1.

FIG. 3 is a partial enlarged diagram of portion A of FIG. 2.

FIG. 4 is a schematic diagram showing inside detailed structure of the portable temperature regulation device of FIG. 1 (where portions of outside walls of a main body, a protrusion portion, and arm portions are omitted).

FIG. 5 is an exploded diagram showing the portable temperature regulation device of FIG. 1 (where portions of outside walls of a main body, a protrusion portion, and arm portions are omitted).

FIG. 6 is an exploded diagram showing the portable temperature regulation device of FIG. 1.

FIG. 7 is a perspective diagram showing a portable temperature regulation device according to a second embodiment of the application.

FIG. 8 is a perspective diagram, taken from a different angle, showing the portable temperature regulation device of FIG. 7.

FIG. 8A is a perspective diagram, taken from another different angle, showing the portable temperature regulation device of FIG. 7.

FIG. 9 is a partial enlarged diagram of portion B of FIG. 8.

FIG. 10 is a perspective diagram of the portable temperature regulation device of FIG. 8.

FIG. 11 is a perspective diagram of the portable temperature regulation device of FIG. 8.

FIG. 12 is a sectioned perspective diagram of the portable temperature regulation device of FIG. 8.

FIG. 13 is a perspective diagram showing a portable temperature regulation device according to a third embodiment of the application.

FIG. 14 is a first exploded diagram of the portable temperature regulation device of FIG. 13.

FIG. 15 is a second exploded diagram of the portable temperature regulation device of FIG. 13.

FIG. 16 is a schematic diagram showing a first air guiding member of the portable temperature regulation device of FIG. 13.

FIG. 17 is a second exploded diagram of the portable temperature regulation device of FIG. 13.

FIG. 18 is a schematic diagram showing a heat dissipating member of the portable temperature regulation device of FIG. 13.

FIG. 19 is a perspective diagram showing a middle portion of a portable temperature regulation device according to a fourth embodiment of the application.

FIG. 20 is an exploded diagram showing the middle portion of the portable temperature regulation device of FIG. 19.

FIG. 21 is a schematic structure diagram of a portable temperature regulation device provided in a fifth embodiment of the application.

FIG. 22 is schematic structure diagram, taken from a different angle, of the portable temperature regulation device shown in FIG. 21.

FIG. 23 is an exploded diagram of the portable temperature regulation device provided in the fifth embodiment of the application.

FIG. 24 is a schematic structure diagram of the portable temperature regulation device shown in FIG. 23, with an outside casing removed.

FIG. 25 is a schematic structure diagram of FIG. 24 taken from a different angle.

FIG. 26 is an exploded diagram of FIG. 24.

FIG. 27 is a schematic diagram of FIG. 26 taken from a different angle.

FIG. 28 is a schematic structure diagram showing a flow directing member provided in the fifth embodiment of the application.

FIG. 29 is a schematic structure diagram of a portable temperature regulation device provided in a sixth embodiment of the application.

FIG. 30 is a schematic structure diagram of an arm portion of the portable temperature regulation device shown in FIG. 29.

FIG. 31 is a schematic exploded structure diagram of the arm portion shown in FIG. 30.

FIG. 32 is a further schematic exploded structure diagram of FIG. 31.

FIG. 33 is a schematic exploded structure diagram of one side of an inside casing of FIG. 32.

FIG. 34 is a schematic structure diagram of a covering member of the sixth embodiment of the application.

FIG. 35 is a schematic diagram of a light diffusion plate of the sixth embodiment of the application.

FIG. 36 is a schematic structure diagram of a base portion of the portable temperature regulation device shown in FIG. 29.

FIG. 37 is a schematic exploded structure diagram of the base portion shown in FIG. 36.

FIG. 38 is a schematic exploded structure diagram of the base portion shown in FIG. 36, taken from a different angle.

FIG. 39 is a schematic exploded structure diagram of the structure of one side of the inside casing of FIG. 38.

FIG. 40 is a schematic cross-sectional view showing the outside casing and the inside casing of the base portion shown in FIG. 36 in a disassembled form.

FIG. 41 is a schematic partial enlarged view of area C of FIG. 40.

FIG. 42 is a schematic cross-sectional view showing the outside casing and the inside casing of the base portion shown in FIG. 40 in an assembled form.

FIG. 43 is schematic structure diagram of the outside casing of the base portion shown in FIG. 36.

FIG. 44 is a schematic perspective diagram of a portable temperature regulation device according to a seventh embodiment of the application.

FIG. 45 is a schematic exploded diagram of the portable temperature regulation device of FIG. 44.

FIG. 46 is another schematic exploded diagram of the portable temperature regulation device of FIG. 44.

FIG. 47 is a further schematic exploded diagram of the portable temperature regulation device of FIG. 44.

FIG. 48 is yet a further schematic exploded diagram of the portable temperature regulation device of FIG. 44.

FIG. 49 is a perspective structure diagram of a portable temperature regulation device according to an eighth embodiment of the application.

FIG. 50 is a first exploded view of the portable temperature regulation device shown in FIG. 49.

FIG. 51 is a second exploded view of the portable temperature regulation device shown in FIG. 49.

FIG. 52 is a schematic structure diagram of a first internal seat of FIG. 51.

FIG. 53 is a third exploded view of the portable temperature regulation device shown in FIG. 49.

FIG. 54 is a schematic structure diagram of the first internal seat of FIG. 53.

FIG. 55 is a schematic perspective structure diagram of a miniature centrifugal impeller provided in a ninth embodiment of the application.

FIG. 56 is another schematic perspective view of the miniature centrifugal impeller shown in FIG. 55.

FIG. 57 is a schematic cross-sectional structure diagram of the miniature centrifugal impeller shown in FIG. 55.

FIG. 58 is a schematic diagram illustrating an effect of projection of a first reinforcing rib and a second reinforcing rib shown in FIG. 57 on a projection plane perpendicular to a first direction.

FIG. 59 is a schematic perspective structure diagram of a portable temperature regulation device provided in a tenth embodiment of the application.

FIG. 60 is a schematic cross-sectional structure diagram of the portable temperature regulation device shown in FIG. 59.

FIG. 61 is a schematic perspective structure diagram of a miniature centrifugal impeller shown in FIG. 59.

FIG. 62 is a schematic structure diagram of the miniature centrifugal impeller shown in FIG. 61 taken from a different viewing angle.

FIG. 63 is a schematic diagram showing a relationship of relative positions of the miniature centrifugal impeller and a housing shown in FIG. 61.

FIG. 64 is a schematic view showing a relationship of related dimensions of the miniature centrifugal impeller and the air inlet opening shown in FIG. 61 in a different way of implementation.

FIG. 65 is a schematic perspective structure diagram of a miniature centrifugal impeller provided in an eleventh embodiment of the application.

FIG. 66 is a schematic perspective structure diagram of the miniature centrifugal impeller shown in FIG. 65 taken from a different viewing angle.

FIG. 67 is a top plan diagram of the miniature centrifugal impeller shown in FIG. 65.

FIG. 68 a schematic cross-sectional diagram of the miniature centrifugal impeller shown in FIG. 67 taken along line I-I.

FIG. 69 a bottom plan diagram of the miniature centrifugal impeller shown in FIG. 65.

FIG. 70 a side elevational diagram of the miniature centrifugal impeller shown in FIG. 65.

FIG. 71 is a schematic perspective exploded structure diagram of a centrifugal fan provided in a twelfth embodiment of the application.

FIG. 72 is a schematic structure diagram of a fan speed regulation circuit provided in a thirteenth embodiment of the application.

FIG. 73 is a schematic diagram of a structure of an embodiment of the fan speed regulation circuit shown in FIG. 72.

FIG. 74 is a schematic diagram of a specific structure of a power supply module in the fan speed regulation circuit provided in the thirteenth embodiment of the application.

FIG. 75 is a schematic diagram of a specific structure of a pulse width modulation chip in the fan speed regulation circuit provided in the thirteenth embodiment of the application.

FIG. 76 is a schematic diagram of a structure of a voltage regulation unit in the fan speed regulation circuit provided in the thirteenth embodiment of the application.

FIG. 77 is a schematic diagram of a structure of a switch unit in the fan speed regulation circuit provided in the thirteenth embodiment of the application.

FIG. 78 is a schematic diagram of a structure of a motor protection unit in the fan speed regulation circuit provided in the thirteenth embodiment of the application.

FIG. 79 is a schematic diagram of a structure of a fan assembly provided in a fourteenth embodiment of the application.

FIG. 80 is a schematic exploded diagram of the structure of the fan assembly provided in the fourteenth embodiment of the application.

FIG. 81 is a schematic structure diagram of a stator of FIG. 80.

FIG. 82 is a schematic structure diagram of a motor bracket of FIG. 81.

FIG. 83 is a schematic view illustrating direction of force applications to two pairs of windings in FIG. 80.

FIG. 84 is a schematic structure diagram of a circuit board of FIG. 80.

FIG. 85 is a schematic exploded structure diagram of another fan assembly provided in a fifteenth embodiment of the application.

FIG. 86 is a schematic structure diagram of a stator of FIG. 85.

FIG. 87 is a schematic structure diagram of a motor bracket shown in FIG. 86.

FIG. 88 is a schematic structure diagram of a portable temperature regulation device provided in a sixteenth embodiment of the application.

FIG. 89 is a schematic exploded structure diagram of the portable temperature regulation device of FIG. 88.

FIG. 90 is a schematic exploded structure diagram of a semiconductor cooler device provided in a seventeenth embodiment of the application.

FIG. 91 is a schematic structure diagram of the semiconductor cooler device shown in FIG. 90, with a first substrate, a second substrate, and an encapsulating member removed.

FIG. 92 is a schematic cross-sectional structure diagram of the semiconductor cooler device shown in FIG. 90.

FIG. 93 is a schematic structure diagram of a portable temperature regulation device provided in an eighteenth embodiment of the application.

FIG. 94 is a schematic cross-sectional structure diagram of the portable temperature regulation device shown in FIG. 93.

FIG. 95 is a schematic exploded structure diagram of the portable temperature regulation device shown in FIG. 93.

FIG. 96 is a schematic diagram illustrating a relationship of dimensions of a temperature conducting member and a semiconductor cooler device shown in FIG. 95.

FIG. 97 is a schematic structure diagram of a portable temperature regulation device provided in a nineteenth embodiment of the application.

FIG. 98 is a schematic partial exploded diagram of the portable temperature regulation device shown in FIG. 97.

FIG. 99 is a schematic cross-sectional diagram of the portable temperature regulation device shown in FIG. 97.

FIG. 100 is a schematic diagram of the portable temperature regulation device shown in FIG. 97, with a fixing plate not installed.

FIG. 101 is a schematic partial exploded view of an arm portion, a temperature regulation unit, and a control assembly shown in FIG. 100.

FIG. 102 is a schematic structure diagram of a portable temperature regulation device provided in a twentieth embodiment of the application, with a fixing plate not installed.

FIG. 103 is a schematic structure diagram of a portable temperature regulation device of a twenty-first embodiment of the application.

FIG. 104 is a first schematic exploded diagram of the portable temperature regulation device of FIG. 103.

FIG. 105 is a second schematic exploded diagram of the portable temperature regulation device of FIG. 103.

FIG. 106 is a third schematic exploded diagram of the portable temperature regulation device of FIG. 103.

FIG. 107 is a schematic exploded diagram of a portable temperature regulation device of a twenty-second embodiment of the application.

FIG. 108 is another schematic exploded diagram of the portable temperature regulation device of the twenty-second embodiment of the application.

FIG. 109 is a schematic structure diagram of a portable temperature regulation device of a twenty-third embodiment of the application.

FIG. 110 is a schematic exploded structure diagram of the portable temperature regulation device of FIG. 109.

FIG. 111 is a schematic exploded diagram of a partial structure of the portable temperature regulation device of FIG. 109.

FIG. 112 is a schematic exploded diagram of the partial structure of the portable temperature regulation device of FIG. 111, taken from a different angle.

FIG. 113 is a schematic structure diagram of the portable temperature regulation device of FIG. 109, taken from a different angle.

FIG. 114 is a cross-sectional diagram of the portable temperature regulation device of FIG. 113.

FIG. 115 is a schematic structure diagram of a portable temperature regulation device provided in a twenty-fourth embodiment of the application.

FIG. 116 is a schematic structure diagram of the portable temperature regulation device of FIG. 115, taken from a different angle.

FIG. 117 is a schematic exploded diagram of a partial structure of the portable temperature regulation device of FIG. 115.

FIG. 118 is a further exploded diagram of the partial structure of the portable temperature regulation device of FIG. 117.

FIG. 119 is a schematic exploded diagram of the partial structure of the portable temperature regulation device of FIG. 118, taken from a different angle.

FIG. 120 is a cross-sectional diagram of the portable temperature regulation device of FIG. 115, taken from a different angle.

FIG. 121 is a schematic structure diagram of a portable temperature regulation device provided in a twenty-fifth embodiment of the application.

FIG. 122 is a schematic exploded structure diagram of the portable temperature regulation device of FIG. 121.

FIG. 123 is a schematic exploded diagram of a partial structure of the portable temperature regulation device of FIG. 122.

FIG. 124 is a schematic exploded structure diagram of the portable temperature regulation device of FIG. 121, taken from a different angle.

FIG. 125 is a cross-sectional diagram of the portable temperature regulation device of FIG. 121, taken from a different angle.

### DETAILED DESCRIPTION OF PREFERRED EMBODIMENTS

In order to have a clearer understanding of the technical features, purposes, and effects of the application, the specific ways of implementation of the application will be described in detail with reference to the accompanying drawings. It should be understood that the directional or positional relationship indicated by “front”, “rear”, “up”, “down”, “left”, “right”, “longitudinal”, “transverse”, “vertical”, “horizontal”, “top”, “bottom”, “inside”, and “outside” as used in the specification is based on the directional or positional relationship shown in the drawings, constructed and operated in a specific orientation, and is only for the convenience of describing the present technical solution, and does not indicate that the device or element referred to must have a specific direction, and thus it cannot be understood as a limitation of the application. It should also be noted that unless otherwise clearly specified and limited, the terms “mount”, “interconnect”, “connect”, “fix”, and “arranged” as used in the specification should be construed in a broad sense, such as being fixedly connected, or detachably connected, or integrated as one piece; it can be directly connected, or indirectly connected through an intermediate medium, and it can be the internal connection or an interactive relationship between two elements. When an element is referred to as being “above” or “under” another element, the element can be “directly” or “indirectly” located above the other element, or there may be one or more intermediate elements. The terms “first”, “second”, “third”, and so on as used in the specification are only for the convenience of describing the technical solution, and cannot be construed as indicating or implying relative importance or implicitly indicating the number of the indicated technical features. Therefore, the features defined as “first”, “second”, and “third” may explicitly or implicitly include one or more of the features. For ordinary technicians in this field, the specific meanings of the above terms used in the present invention can be understood according to specific circumstances.

In the following description, specific details such as specific system structures and technologies are provided for the purpose of illustration rather than limitation, so as to provide a thorough understanding of the embodiments of the application. However, it should be clear to those skilled in the art that the application can also be implemented in other embodiments without these specific details. In other cases, detailed descriptions of well-known systems, devices, circuits, and methods are omitted to avoid unnecessary details that obscure the description of the application.

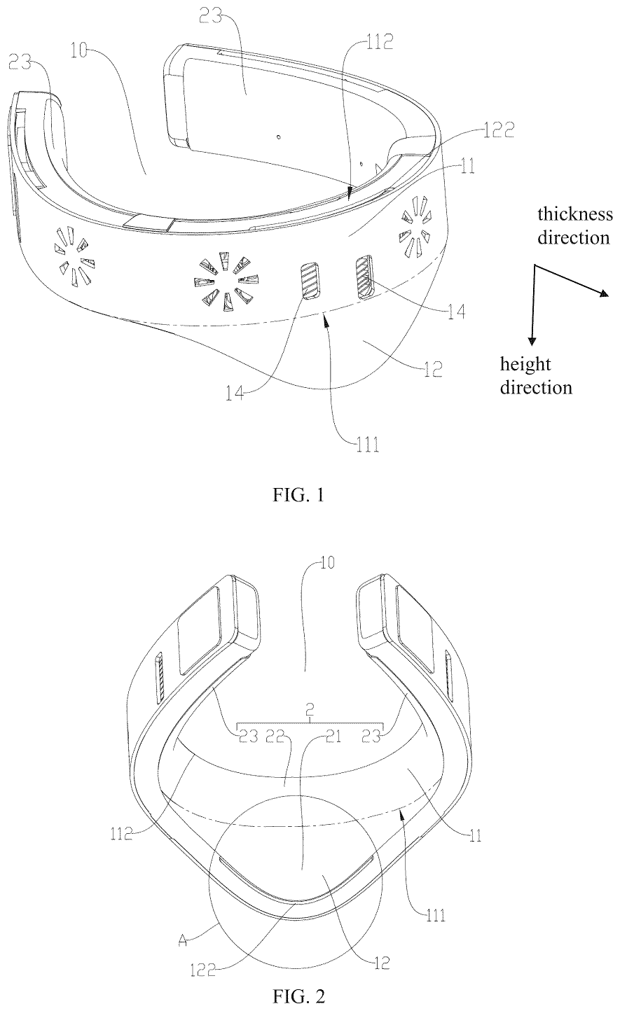

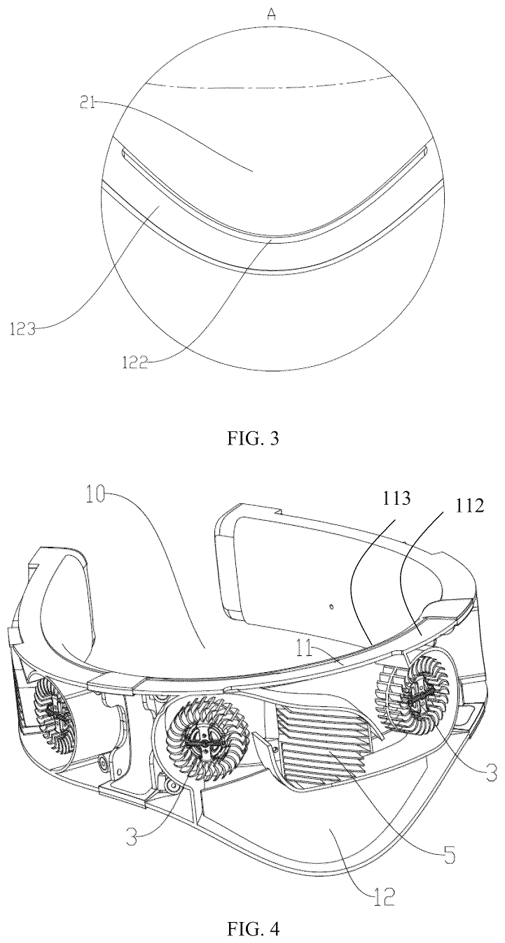

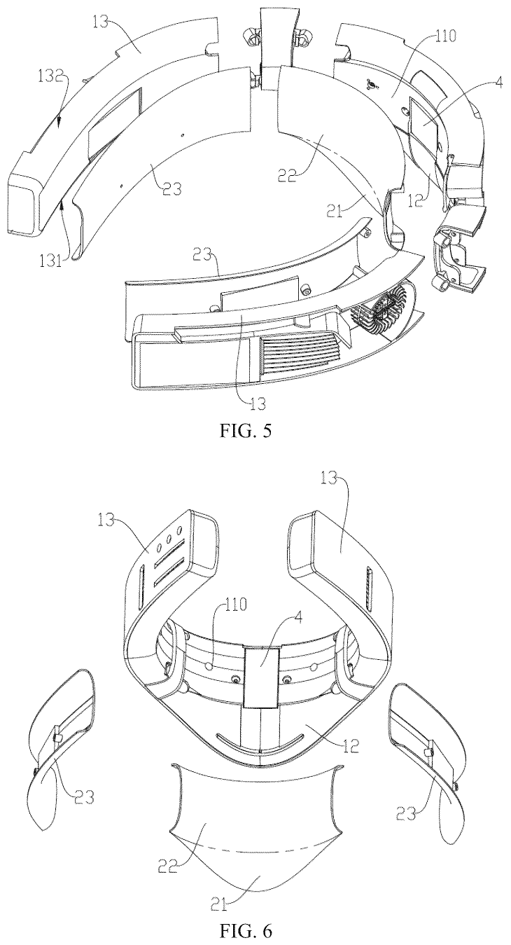

Referring to FIGS. 1-6, a portable temperature regulation device in accordance with an embodiment of the present disclosure defines a wear space 10. The portable temperature regulation device comprises a main body 11, a protrusion portion 12 arranged on the main body 11, and a temperature conducting member 2 at least partly arranged on the protrusion portion 12 for contacting with a human body. The main body 11 has a first side 111, and the protrusion portion 12 is arranged at the first side 111 and extends/protrudes in a direction away from the first side 111.

Specifically, the portable temperature regulation device is a neck-mounted temperature regulation device. The main body 11 is of a U-shaped structure having a free end or an enclosed circular structure having no free end. The main body 11 can individually defines the wear space 10, or the main body 11 and the protrusion portion 12 jointly define the wear space 10. The wear space 10 enables wearing on a portion of the human body, such as the human neck. The temperature conducting member 2 is arranged on a surface of the protrusion portion 12 and faces the wear space 10. The temperature conducting member 2 is preferably made of a material of high thermal conduction efficiency, such as metal materials, for example aluminum materials. With the portable temperature regulation device worn, through the wear space 10, on the human neck, the first side 111 of the main body 11 is in contact with the human shoulders to serve as a supporting surface. As the protrusion portion 12 is arranged on the first side 111 and extends and protrudes in the direction away from the first side 111, the protrusion portion 12 may reach into the collar such that the temperature conducting member 2 is allowed to contact the human back area.

The portable temperature regulation device may be extended, through the protrusion portion 12, toward a collar covering the human back and may be even extended into the collar to contact the human back with the temperature conducting member 2 arranged on the protrusion portion 12, so as to allow the temperature conducting member 2 to conduct cold or heat toward the human back, realizing temperature regulation for the back area covered by the collar, meeting the needs of regulation on the human back for cold compress for cooling or hot compress for massaging, improving the user's experience of use.

The temperature conducting member 2 comprises a first portion 21 arranged on one side surface of the protrusion portion 12 facing the wear space 10. The first portion 21 is extended to one side of the protrusion portion 12 away from the main body 11, so that one side of the protrusion portion 12 away from the main body 11 can conduct temperature when in contact with the human body.

One side of the protrusion portion 12 that is away from the main body 11 is provided with a back-side air outlet opening 122, and the first portion 21 is arranged to avoid the back-side air outlet opening 122 or the first portion 21 is provided with a through hole corresponding to the back-side air outlet opening 122. That is, the first portion 21 does not block the air outlet opening 122.

In the instant embodiment, when the temperature conducting member 2 is conducting cold toward the human back, to increase the effect of cooling of the human back, the back-side air outlet opening 122 is provided on the lower side of the protrusion portion 12 away from the main body 11, and the first portion 21 is arranged to avoid the back-side air outlet opening 122 or the first portion 21 is provided with the through hole corresponding to the back-side air outlet opening 122. The first portion 21 and the back-side air outlet opening 122 operate independently, and the first portion 21 does not block air exiting from the back-side air outlet opening 122. The first portion 21 and the back-side air outlet opening 122 do not interfere with each other. Through the back-side air outlet opening 122, air inside the protrusion portion 12 can be blown toward the back of the user to achieve a cooling effect. The back-side air outlet opening 122 may also named as the first air outlet opening 122 in this embodiment.

The temperature conducting member 2 comprises a second portion 22 arranged on an inner side surface of the main body 11 facing the wear space 10. The first portion 21 and the second portion 22 are separate or integrated. Specifically, when the first portion 21 and the second portion 22 are separate, it means the first portion 21 and the second portion 22 are two parts separately formed; and when the first portion 21 and the second portion 22 are integrated, it means the first portion 21 and the second portion 22 are one part integrally formed. Thus, temperature can be conducted to the neck through the second portion 22 of the temperature conducting member 2 and the protrusion portion 12 is extended into the collar so as to conduct temperature through the first portion 21 of the temperature conducting member 2 toward the human back, and the area for conducting temperature to the human body is expanded, and a wider range of temperature regulation can be achieved.

When the first portion 21 and the second portion 22 are integrated, the first portion 21 smoothly transits to the second portion 22. Specifically, the contact surface of the first portion 21 is flush with the contact surface of the second portion 22, so that the portable temperature regulation device is more ergonomic. When the human back is in contact with the first portion 21 and the human neck is in contact with the second portion 22, the feeling of touch of the human body for contacting with the temperature conducting member 2 is more comfortable.

The main body 11 comprises a second side 112 opposite to the first side 111, and the second portion 22 is extended to the second side 112. The second side 112 is also named as upper side and the first side 111 is also named as lower side.

The main body 11 comprises a base portion 110 and arm portions 13 respectively arranged at two ends of the base portion 110. The base portion 110 and the two arm portions 13 jointly define the wear space 10. The temperature conducting member 2 comprises a third portion 23 arranged on a side surface of the arm portions 13 facing the wear space 10. In the instant embodiment, there are two third portions 23, which are respectively arranged on the two arm portions 13. The second portion 22 is contactable with the back side of the human neck, and the two third portions 23 are respectively set in contact with the left and right sides of the human neck. The second portion 22 and the third portion 23 are separate or integrated. Specifically, when the second portion 22 and the third portion 23 are separate, it means the second portion 22 and the third portion 23 are two separate parts; and when the second portion 22 and the third portion 23 are integrated, it means the second portion 22 and the third portion 23 are one single integrally formed part. Thus, through the third portions 23 of the temperature conducting member 2, temperature is conducted, through contact, toward sides of the human neck, so that the area for conducting temperature to the human body is further expanded, and an even wider range of temperature regulation can be achieved.

The base portion 110 comprises a first side and a second side opposite to the first side, and the arm portion 13 comprises a third side 131 and a fourth side 132. The third side 131 is on the same side as the first side, and the third side 131 of the arm portion 13 and the first side of the base portion 110 cooperatively form the first side 111 (for example: upper side) of the main body 11. The fourth side 132 of the arm portion 13 is on the same side as the second side of the base portion 110, and the fourth side 132 of the arm portion 13 and the second side of the base portion 110 cooperatively form the second side 112 (for example: lower side) of the main body 11. The third portion 23 is extended to the third side 131 and/or the fourth side 132 in the height direction of main body 11 or the axial direction of the wear space 10. That is, the upper end of the third portion is substantially flush with the third side 131 and/or the lower end of the third portion is substantially flush with the fourth side 132. Preferably, the third portion 23 has a height substantially equal to that of the arm portion 13. The upper end of the third portion 23 is substantially flush with the upper end/side of the arm portion 13 and the lower end of the third portion 23 is substantially flush with the lower end/side of the arm portion 13.

In the instant embodiment, the third portions 23 are arranged to substantially flush with the third sides 131 and/or the fourth sides 132 of the arm portions 13. On the one hand, the contact area with the human body neck can be significantly increased, and on the other hand, after the human body wears the portable temperature regulation device of the application, when the human body moves the head, such as turning the head angle, the third portion 23 can constantly keep contact with the human neck, making the experience of conducting temperature through the temperature conducting member 2 to the human body better.

An outer contour of the protrusion portion 12 away from the main body 11 is a curved structure. As such, the shape of the protrusion portion 12 conforms to ergonomics and has a rounded configuration, so that during a course of wearing, the feeling is more comfortable for the protrusion portion 12 contacting or even hitting the human body, not easy to be scratched or pricked. Further, compared with a linear structure, the curved structure can reduce the contact area of a user contacting the side of the protrusion portion 12 away from the main body 11, thereby further increasing the wearing comfort.

The curved structure comprises an inclined surface 123 that is inclined from the side away from the wear space 10 toward the side close to the wear space 10. The inclined surface 123 is configured to prevent the end surface of the protrusion portion 12 from abutting against the back of the human body to cause discomfort to the user when the user raises his head. Thus, the arrangement of the inclined surface 123 makes the configuration of the protrusion portion 12 more ergonomic and the shape more rounded, thereby improving the wearing comfort.

The portable temperature regulation device comprises a temperature regulation member 4 arranged on the main body 11, the arm portions 13, and/or the protrusion portion 12 for cooling and/or heating. The temperature conducting member 2 is in thermal conduction contact with the temperature regulation member 4 to conduct the temperature of the temperature regulation member 4 to the human body. Specifically, the temperature regulation member 4 can be a thermoelectric cooler such as a semiconductor cooling sheet, and when supplied with electrical power, two opposite ends thereof respectively form a cold end and a hot end, and switching the direction of electrical current, the cold end and the hot end are switched with each other. The temperature conducting member 2 conducts cold or heat to the human body.

The portable temperature regulation device comprises a fan 3 arranged in the main body 11 or the protrusion portion 12 and a heat dissipating member 5 arranged in the main body 11 or the protrusion portion 12. One end of the temperature regulation member 4 is in thermal conduction contact with the heat dissipating member 5, and another end of the temperature regulation member 4 is in thermal conduction contact with the temperature conducting member 2. One side (for example outer side) of the main body 11 or the protrusion portion 12 opposite to the wear space 10 is provided with a heat dissipation opening 14, and an airflow generated by the fan 3, after passing through and subjected to heat exchange with the heat dissipating member 5, is blown out through the heat dissipation opening 14 to fulfill heat dissipation. In the embodiment of FIG. 4, the fan 3 and the temperature regulation member 4 are arranged in the main body 11, and the side of the main body 11 away from the wear space 10 is provided, at a location corresponding to the heat dissipating member 5, with the heat dissipation opening 14, so that airflow of the fan 3 is subjected to heat exchange with the heat dissipating member 5 when passing through the heat dissipating member 5 and is then blown out through the heat dissipation opening 14 to achieve heat dissipation. It is noted that it is only when the temperature conducting member 2 needs to conduct cold that the heat dissipating member 5 and the fan 3 need to cooperate to dissipation heat from the hot end of the temperature regulation member 4. The protrusion portion 12 comprises an interior space which is in communication with the first air outlet opening 122 and an air exit opening of the fan 3 such that part of the airflow generated by the fan 3 can flow into the interior space of the protrusion portion 12 via the air exit opening and exit the interior space of the protrusion portion 12 through the first air outlet opening 122. Specifically, the fan 3 is a centrifugal fan including an impeller. Another part of the airflow generated by the fan 3 flows into another space/channel, where the heat dissipating member 5 is accommodated, via another air exit opening of the fan 3. Preferably, in this embodiment, multiple other fans 3 are provided. The side 112 of the main body 11 defines second air outlet openings 113 corresponding to the other fans 3 such that airflow generated by the other fans 3 can be blown to the neck of the user via the second air outlet openings 113.

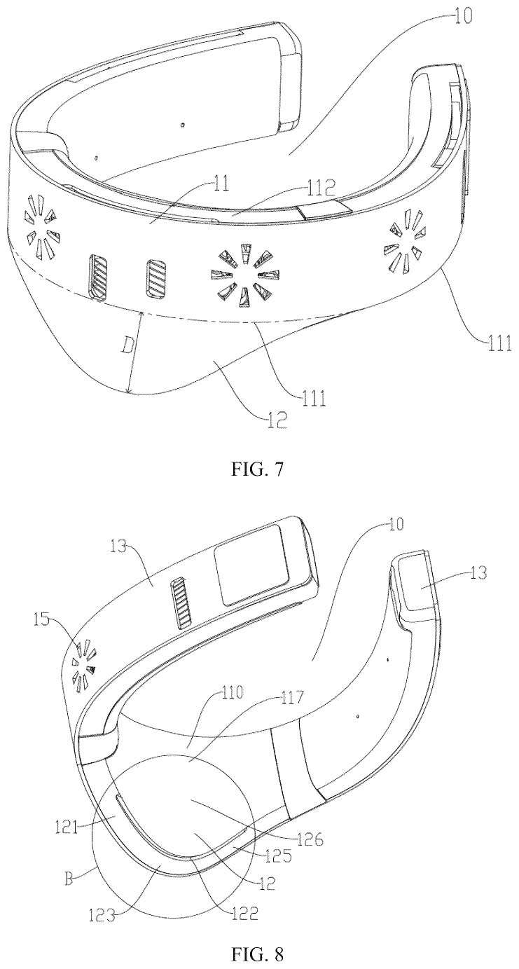

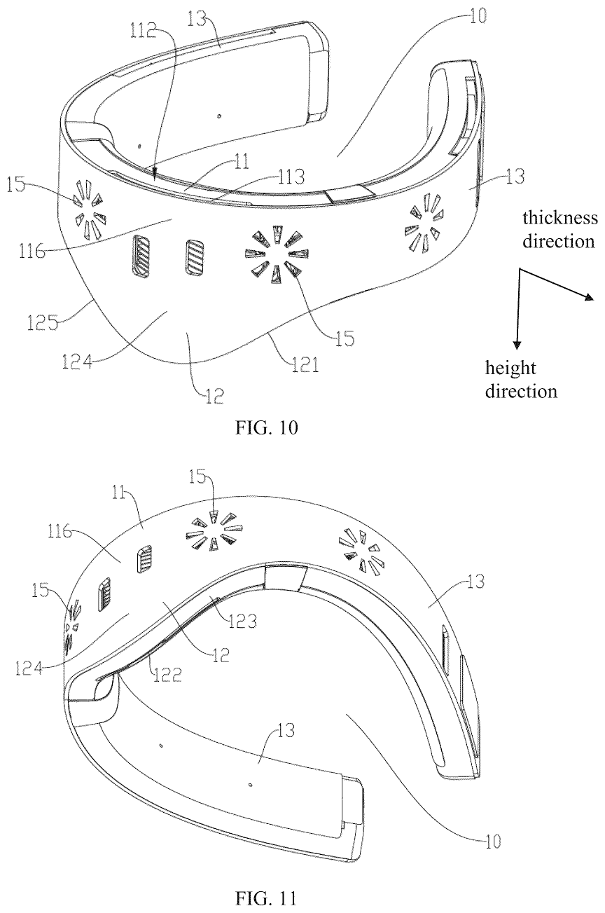

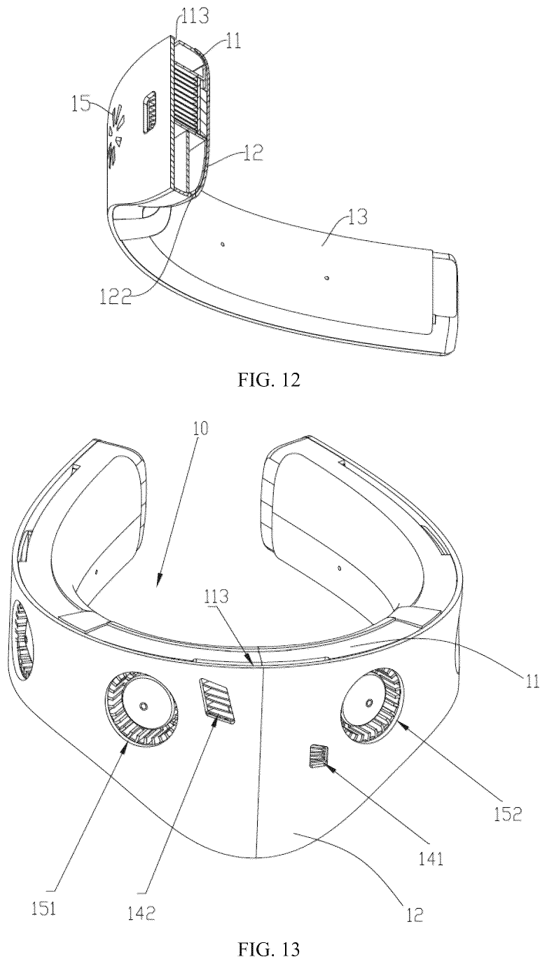

Referring to FIGS. 7-12, a portable temperature regulation device according to a second embodiment of the application is shown, which is basically similar to the portable temperature regulation device of the first embodiment:

In the instant embodiment, the extension distance of the protrusion portion 12 (referring to D in FIG. 7) is greater than 15 mm, so that the first air outlet opening 122 defined in the side of the protrusion portion 12 opposite to the second side 112 can blow air toward the human back, thereby meeting the needs for temperature lowering adjustment of the human back to improve the cooling experience of the user.

Further, the extension distance of the protrusion portion 12 is 20 mm-50 mm, for example 20 mm, 30 mm, 40 mm, or 50 mm. The extension distance allows the protrusion portion 12 to effectively extend into the collar to blow air toward and cool the back of the user without affecting the activities of the user wearing the portable temperature regulation device. For example, after the user wears the portable temperature regulation device on the human neck through the wear space 10, the protrusion portion 12 will not prevent the user from tilting the head backwards.

Further, preferably, the extension distance of the protrusion portion 12 is 30 mm-40 mm, for example 30 mm, 33 mm, 36 mm, or 40 mm. The extension distance allows the protrusion portion 12 to more effectively extend into the collar to blow air toward and cool the back, and also, the extension distance is more suitable, and the wearing experience of the user is more comfortable.

An outer contour of the side of the protrusion portion 12 opposite to the second side 112 has a curved configuration.

In the instant embodiment, during the use of the portable temperature regulation device, since the end side of the protrusion portion 12 opposite to the second side 112 is in contact with the user's back, making the end side of the protrusion portion 12 opposite to the second side 112 a curved structure makes the shape thereof more rounded and conforming to ergonomic design. On the one hand, the user can feel more comfortable when in contact with the end side of the protrusion portion 12 opposite to the second side 112, without feeling scratched or stung, improving wear comfort. On the other hand, compared with a linear structure, the curved structure can reduce the contact area when the user is in contact with the end side of the protrusion portion 12 opposite to the second side 112, thereby further improving wear comfort.

Further, the curved structure comprises a first slanted wall 121 and a second slanted wall 125. The first slanted wall 121 and the second slanted wall 125 are connected to each other and the distance between the two gradually decreases in the direction away from the second side. That is, the width of the protrusion portion 12 gradually decreases in the height direction away from the second side of the main body 11.

In the instant embodiment, one end of the first slanted wall 121 is connected to the first side 111 of the main body 11, and another end of the first slanted wall 121 is connected to one end of the second slanted wall 125. Another end of the second slanted wall 125 is also connected to the first side 111. Also, the first slanted wall 121 and the second slanted wall 125 are connected to each other and the distance between the two gradually decreases in the direction away from the second side, so that the first slanted wall 121 and the second slanted wall 125 together form a “V”-shaped structure with a round transition. Compared with a linear structure, this arrangement can reduce the contact area when the user is in contact with the end side of the protrusion portion 12 opposite to the second side 112, thereby further improving wear comfort.

Further, the first air outlet opening 122 is arranged on the first slanted wall 121 and the second slanted wall 125.

In the instant embodiment, the first air outlet opening 122 is provided on both of the first slanted wall 121 and the second slanted wall 125, and one end of the first air outlet opening 122 is arranged on the first slanted wall 121, while the other end is on the second slanted wall 125, so that the first air outlet opening 122 has also a “V”-shape. This arrangement makes air exiting from the first air outlet opening 122 exhibit a diffusing way of air egress, increasing the air exiting area of the first air outlet opening 122, thereby increase the air blowing area on the human back. Of course, the number of the first air outlet opening 122 can be two, of which one first air outlet opening 122 is arranged on the first slanted wall 121 and another one first air outlet opening 122 is arranged on the second slanted wall 125.

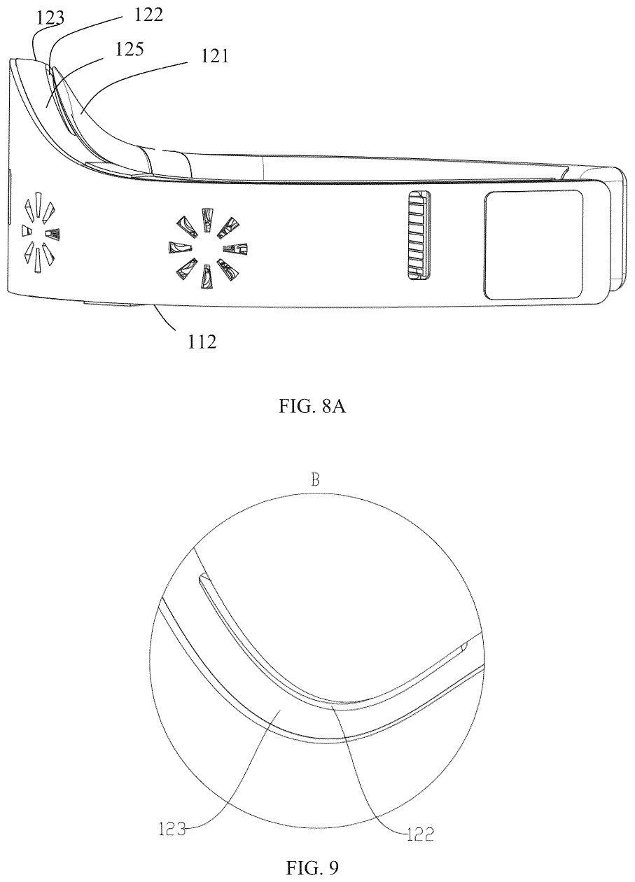

Further, the curved structure comprises an inclined end surface 123, and the first air outlet opening 122 is defined at the inclined end surface 123. Preferably, the first air outlet opening 122 is defined at the inner side of the inclined end surface 123 close to the wear space 10. In a thickness direction from the outer side away from the wear space 10 to the inner side close to the wear space 10, the inclined end surface 123 inclines in a direction toward the second side 112 of the main body 11, as shown in FIG. 8A. Providing the first air outlet opening 122 at the inclined end surface 123 helps prevent the user's back skin from blocking the first air outlet opening 122 to hinder air exiting from the first air outlet opening 122 when the user is in contact with the end of the protrusion portion 12 opposite to the second side 112, thereby increasing the reliability of air exiting. The inclined end surface 123 also functions to prevent the end of the protrusion portion 12 from contacting and hitting the back of the user to cause discomfort to the user when the user raises the head. Therefore, the arrangement of the inclined surface 123 makes the configuration of the protrusion portion 12 more ergonomic and the appearance more rounded, improving wear comfort.

Further, the first air outlet opening 122 is arranged at the edge of the inclined surface 123 close to the wear space 10. Through such a positional arrangement, air blowing out of the first air outlet opening 122 can be guided along the surface of the human back, further improving the cooling effect of the application.

The main body 11 comprises a base portion 110 and arm portions 13 respectively arranged at two ends of the base portion 110. The base portion 110 and the two arm portions 13 jointly define the wear space 10. The base portion 110 and the protrusion portion 12 are integrally formed as a one-piece structure.

In the instant embodiment, the base portion 110 and the protrusion portion 12 may be integrally formed as a one-piece structure. The integrally formed one-piece structure can reduce the assembling and positioning process between the base portion 110 and the protrusion portion 12, helping improve the production yield rate and reduce production cost of the portable temperature regulation device of the application.

Further, the main body 11 comprises a main body outside wall 116 opposite to the wear space 10 in a thickness direction. The protrusion portion 12 comprises a protrusion portion outside wall 124 opposite to the wear space 10 in the thickness direction. The main body outside wall 116 smoothly transitions to the protrusion portion outside wall 124. When the neck-mounted temperature regulation device is in use, the protrusion portion 12 can extend into the collar so that air can be blown toward the back of the user from the protrusion portion 12, fulfilling temperature regulation of the back. Since the main body outside wall 116 smoothly transitions to the protrusion portion outside wall 124, the protrusion portion 12 can easily and smoothly extend into the collar of the user to blow air to the back of the user, not easily hindered by the shape of the main body outside wall 116 and the protrusion portion outside wall 124, and also, the collar is propped up to a small extent, not too abrupt, and has better concealment. The main body outside wall 116 is flush with the protrusion portion outside wall 124, so that the protrusion portion 12 can be easily and smoothly extended into the collar as much as possible, and also, the collar is propped up to an extent as small as possible, and is not abrupt, exhibiting better concealment.

The main body 11 comprises a main body inside wall 117 facing the wear space 10 in the thickness direction. The protrusion portion 12 comprises a protrusion portion inside wall 126 facing the wear space 10 in the thickness direction. The main body inside wall 117 smoothly transitions to the protrusion portion inside wall 126, so that when the neck-mounted temperature regulation device is set on the neck, the neck feels comfortable in contact with the main body inside wall 117 and the protrusion portion inside wall 126.

One or more air inlet openings 15 that allow air to enter the portable temperature regulation device are defined in the outside wall 116 of the main body 11 or the outside wall 124 of the protrusion portion 12 opposite to the wear space 10. One or more fans 3 are arranged in the main body 11 or the protrusion portion 12. The fans 3 are arranged to correspond to the air inlet openings 15 and the first air outlet opening 122. The fan functions to generate accelerated airflows which enter the portable temperature regulation device from the air inlet openings 15 and flow toward the first air outlet opening 122 to be guided out by the first air outlet opening 122.

Further, a second air outlet opening 113 is arranged on the second side 112 of the main body 11 such that parts of the airflows generated by the fans 3 can be blown out via the second air outlet opening 113.

In the instant embodiment, the second side 112 of the main body 11 is provided with the second air outlet opening 113 to allow parts of the airflows to be blown out therethrough. The second air outlet opening 113 may allow air to be blown toward the neck or the head. When the portable temperature regulation device is worn on the neck, the protrusion portion 12 extends toward the back of the human body, allowing the first air outlet opening 122 to precisely blow air toward the back of the user for cooling, and at the same time, the second air outlet opening 113 allow air to be blown toward the neck of user for cooling. Namely, the portable temperature regulation device can effectively blow air toward the human back, while also blowing air toward the human neck, thereby improving the practical use of the portable temperature regulation device, and also bringing a better use experience to the user.

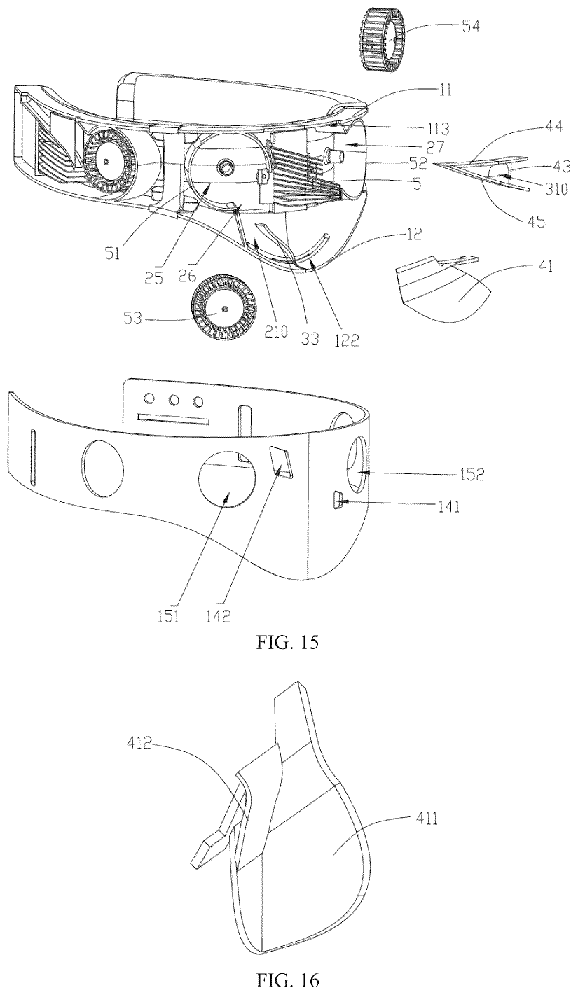

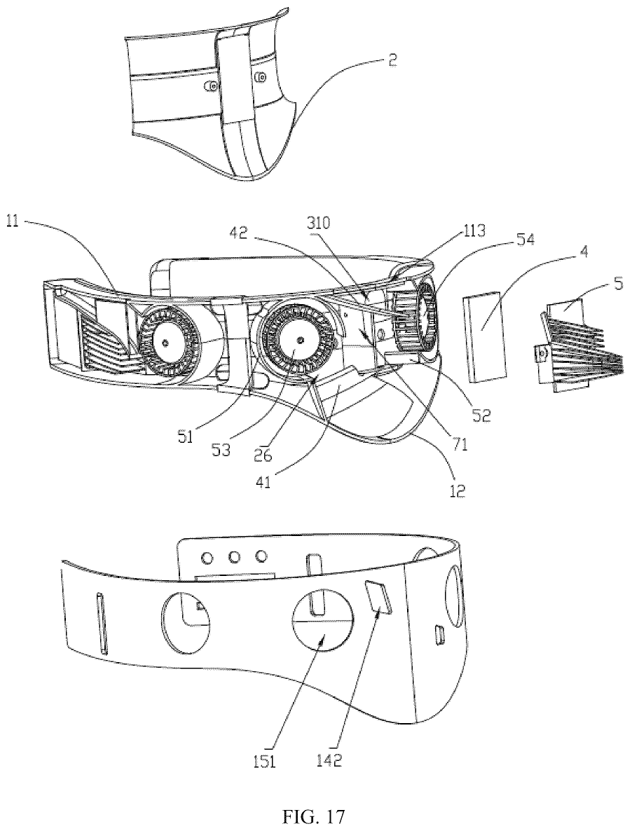

As shown in FIGS. 13-18, further, a middle portion of the main body 11 and the protrusion portion 12 together constitute a middle portion of the portable temperature regulation device. A first air channel 210 and a second air channel 310 are provided in the interior of the middle portion. The first air channel 210 communicates with the first air outlet opening 122, and the second air channel 310 communicates with the second air outlet opening 113. The first air channel 210 can independently supply air to the first air outlet opening 122, and the second air channel 310 can independently supply air to the second air outlet opening 113, so as to optimize the internal air supply structure of the portable temperature regulation device. After the airflow that forms wind is regulated by the independent air supply structures of the first air channel 210 and the second air channel 310, the airflow can be smoothly blown out to the outside through the first air outlet opening 122 and the second air outlet opening 113.

Further, the portable temperature regulation device comprises a first air guiding member 41. The first air guiding member 41 is arranged in the interior of the middle portion. The first air guiding member 41 and an inside wall of the middle portion form the first air channel 210. In the production process, to reduce the difficulty of production of the first air channel 210, the first air guiding member 41 can be fixed in the interior of the middle portion by means of mounting. In other embodiments, the first air guiding member 41 is integrally formed with the inside wall of the middle portion, and the first air guiding member 41 and the inside wall of the middle portion form an air channel to achieve an effect of guiding airflow.

Further, the first air guiding member 41 comprises an air guide plate 411 and a protection plate 412, which are arranged to form an included angle. The air guide plate 411 is connected to the protection plate 412, and the air guide plate 411 is hermetically connected to the side wall of the protrusion portion 12 opposite to the second air outlet opening 113, and the protection plate 412 is hermetically connected to the side wall of the protrusion portion 12 facing the wear space 10, so that no air leakage may occur between the two ends of the first air channel 210, and airflow can be effectively guided toward the first air outlet opening 122. Specifically, one end of the air guide plate 411 away from the first air outlet opening 122 is expanded outward to form a horn-like entrance, so that airflow, after entering the entrance, can be compressed by the first air channel 210 that is located away from the entrance, further making the airflow more concentrated when flowing toward the first air outlet opening 122, thereby making the airflow stronger in flowing out of the first air outlet opening 122.

Further, the side of the air guide plate 411 opposite to the first air channel 210, the side of the protection plate 412 opposite to the first air channel 210, and the side wall of the middle portion together form an accommodation compartment for mounting a circuit board, in order to prevent the circuit board from interfering with the airflow flowing through the first air channel.

In the instant embodiment, the middle portion is provided with an air inlet opening 15. A partition member 33 is arranged in the first air channel 210. The partition member 33 divides the first air channel 210 into a first sub air channel and a second sub air channel. One end of the first sub air channel and one end of the second sub air channel are both in communication with the air inlet opening 15, and the other end of the first sub air channel and the other end of the second sub air channel are both in communication with the first air outlet opening 122. The first air outlet opening 122 is of a flat elongated shape, making the air exiting from the first air outlet opening 122 more uniform. The partition member 33 dividing the first air channel 210 into the first sub air channel and the second sub air channel makes the air exiting volume more uniform at each position of the first air outlet opening 122 when the air is eventually blown out of the first air outlet opening 122.

In the instant embodiment, the portable temperature regulation device comprises a second air guiding member 42. The second air guiding member 42 is arranged in the interior of the middle portion. The second air guiding member 42 comprises a bottom plate 43, a first side plate 44, and a second side plate 45. The first side plate 44 and the second side plate 45 are respectively connected to opposite sides of the bottom plate 43, and the first side plate 44 and the second side plate are arranged in a “V” shape. The bottom plate 43, the first side plate 44, the second side plate 45, and the inside wall of the middle portion form the second air channel 310. During the production process, to reduce the difficulty of production of the second air channel 310, the second air guiding member 42 can be formed or assembled and then fixed in the interior of the middle portion by means of mounting. In other embodiments, the second air guiding member 42 is integrally formed with the inside wall of the middle portion, and the second air guiding member 42 and the inside wall of the middle portion form an air channel to achieve an effect of guiding airflow.

As shown in FIGS. 13-17, in a third embodiment of the application, compared with the second embodiment, further, in the instant embodiment, the middle portion is provided with an air inlet opening 15. The air inlet opening 15 comprises a first air inlet opening 151 and a second air inlet opening 152. The middle portion is provided, in the interior thereof, with a first volute 51, a second volute 52, a first fan 53, and a second fan 54. The first volute 51 surrounds and defines a first mounting compartment 25 in communication with the first air inlet opening 151. The first volute 51 is provided with a first opening 26 in communication with the first mounting compartment 25. The first opening 26 communicates with the first air channel 210. The first fan 53 is arranged in the first mounting compartment 25. The second volute 52 surrounds and defines a second mounting compartment 27 in communication with the second air inlet opening 152. The second volute 52 is provided with a second opening 28 in communication with the second mounting compartment 27. The second opening 28 communicates with the second air channel 310. The second fan 54 is arranged in the second mounting compartment 27. With the first volute 51 and the second volute 52 respectively separating the first fan 53 and the second fan 54, the first fan 53 and the second fan 54 do not interfere with each other during operation.

When the first fan 53 is in operation, the first fan 53 draws in air from the first air inlet opening 151 and converts the air into airflow in the first mounting compartment 25, and the airflow is blown toward the first air outlet opening 122 through the first air channel 210, so that the user can feel the coolness. Similarly, when the second fan 54 is in operation, the second fan 54 draws in air from the second air inlet opening 152 and converts the air into airflow in the second mounting compartment 27, and the airflow is blown toward the second air outlet opening 113 through the second air channel 310, so that the user can feel the coolness. That is, the first mounting compartment 25 individually uses the first air inlet opening 151 to take in air, and the second mounting compartment 27 individually uses the second air inlet opening 152 to take in air. When the first air inlet opening 151 is blocked by foreign objects, the second air inlet opening 152 can still normally supply air to the second mounting compartment 27, and the portable temperature regulation device can still blow air through the second air outlet opening 113 for operation. Similarly, when the second air inlet opening 152 is blocked by foreign objects, the first air inlet opening 151 can still normally supply air to the first mounting compartment 25, and the portable temperature regulation device can still blow air through the first air outlet opening 122 for operation. Thus, at least one of the first air outlet opening 122 and the second air outlet opening 113 of the portable temperature regulation device can operate normally.

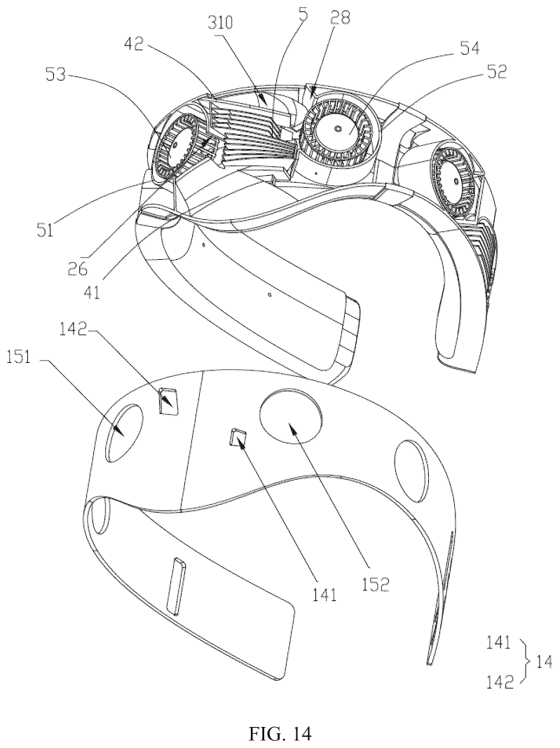

As shown in FIGS. 14-18, further, the portable temperature regulation device comprises a heat dissipating member 5, a temperature conducting member 2, and a temperature regulation member 4. An air channel 71 is arranged in the interior of the middle portion. The air channel 71 is located between the first air channel 210 and the second air channel 310. The heat dissipating member 5 is arranged in an interior of the air channel 71. One side of the middle portion opposite to the wear space 10 is provided with a heat dissipation opening 14 with which the air channel 71 communicates. The temperature conducting member 2 is fixed on the side of the middle portion facing the wear space 10. The temperature regulation member 4 is sandwiched between the heat dissipating member 5 and the temperature conducting member 2 and in thermal conduction connection with the heat dissipating member 5 and the temperature conducting member 2. The temperature regulation member 4 is a semiconductor cooling sheet. When the temperature regulation member 4 cools the temperature conducting member 2, the temperature regulation member 4 correspondingly generates a large amount of heat. The heat is transferred to the heat dissipating member 5 which dissipates heat to the outside of the portable temperature regulation device through the heat dissipation opening 14. When the portable temperature regulation device is worn around the neck, the portable temperature regulation device can not only blow air toward the human body for cooling through the first air outlet opening 122 and the second air outlet opening 113, but also transfers cold to the neck through the temperature conducting member 2 fitting and contacting the neck, thereby making the portable temperature regulation device exhibiting multiple functions for cooling the human body to further improve the utilization of the portable temperature regulation device.

In the instant embodiment, the heat dissipation opening 14 comprises a first heat dissipation opening 141 and a second heat dissipation opening 142. The heat dissipating member 5 divides the air channel 71 into a first air channel and a second air channel. One end of the first air channel is in communication with the first opening 26, and the other end of the first air channel is in communication with the first heat dissipation opening 141. One end of the second air channel is in communication with the second opening 28, and the other end of the second air channel is in communication with the second heat dissipation opening 142. And, the flow direction of airflow passing the first air channel is opposite to the flow direction of airflow passing the second air channel. Specifically, the first air channel and the first air channel 210 separate the airflow blown out of the first opening 26 into two streams, one of the streams being blown out of the first air outlet opening 122 after passing through the first air channel 210 for blowing air to the human back, another one of the streams being blown out of the first heat dissipation opening 141 through the first air channel. When the airflow flows through the first air channel, the airflow takes the heat away from the heat dissipating member 5 to flow out of the first heat dissipation opening 141, so that the heat dissipating member 5 may have heat dissipated and become cooled. The second air channel and the second air channel 310 separate the airflow blown out of the second opening 28 into two streams, one of the streams being blown out of the second air outlet opening 113 after passing through the second air channel 310 for blowing air toward the human neck, another one of the streams being blown out of the second heat dissipation opening 142 through the second air channel. When the airflow flows through the second air channel, the airflow takes heat away from the heat dissipating member 5 to flow out of the second heat dissipation opening 142, so that the heat dissipating member 5 may have heat dissipated and become cooled. Thus, the heat dissipating member 5 can absorb heat from the temperature regulation member 4 continuously.

In the instant embodiment, the heat dissipating member 5 comprises first heat dissipation fins 511 and second heat dissipation fins 512. The first heat dissipation fins 511 and the second heat dissipation fins 512 are both plural. Between two adjacent first heat dissipation fins 511, a first heat dissipation gap 73 is formed. One end of the first heat dissipation gap 73 away from the first opening 26 is in communication with the first heat dissipation opening 141. Between adjacent second heat dissipation fins 512, a second heat dissipation gap 74 is formed. One end of the second heat dissipation gap 74 away from the second opening 28 is in communication with the second heat dissipation opening 142. Air inlet end of the first heat dissipation gap 73 is arranged opposite to air inlet end of the second heat dissipation gap 74. The first heat dissipation gap 73 guides airflow blown out of the first opening 26 toward the first heat dissipation opening 141 to blow out thereof, and the second heat dissipation gap 74 guides airflow blown out of the second opening 28 toward the second heat dissipation opening 142 to blow out thereof. The direction of airflow blown out of the first opening 26 flowing through the first heat dissipation gap 73 is opposite to the direction of airflow blown out of the second opening 28 flowing through the second heat dissipation gap 74. Through the plurality of first heat dissipation fins 511 and the plurality of first heat dissipation gaps 73, and the plurality of second heat dissipation fins 512 and the plurality of second heat dissipation gaps 74, the area of the heat dissipating member 5 contacting the airflow as a whole is increased, and the heat dissipation capacity of the heat dissipating member 5 is enhanced. The end of the first heat dissipation gap 73 close to the first opening 26 is wider than the other end of the first heat dissipation gap 73 away from the first opening 26, which facilitates to accelerate the airflow when the airflow passes through the first heat dissipation gap 73.

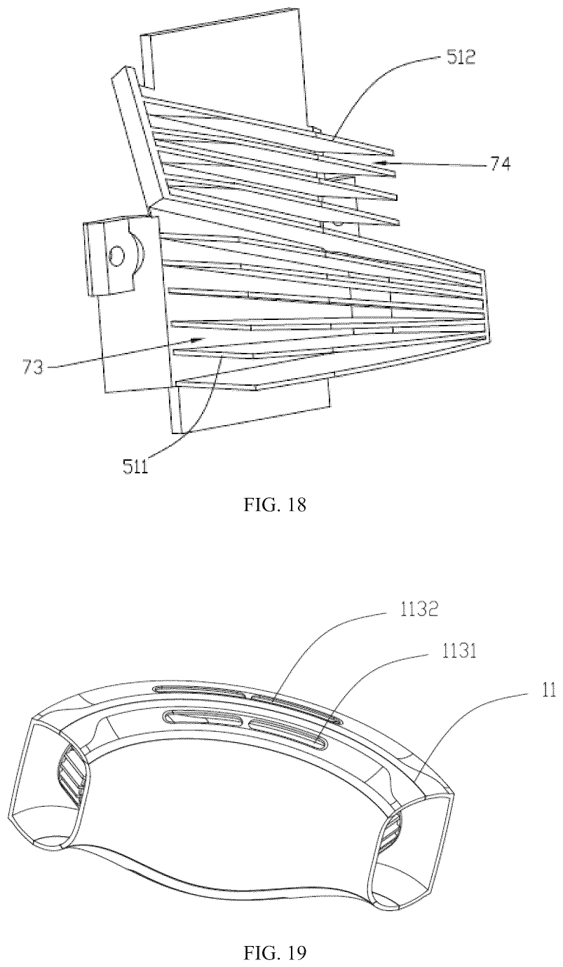

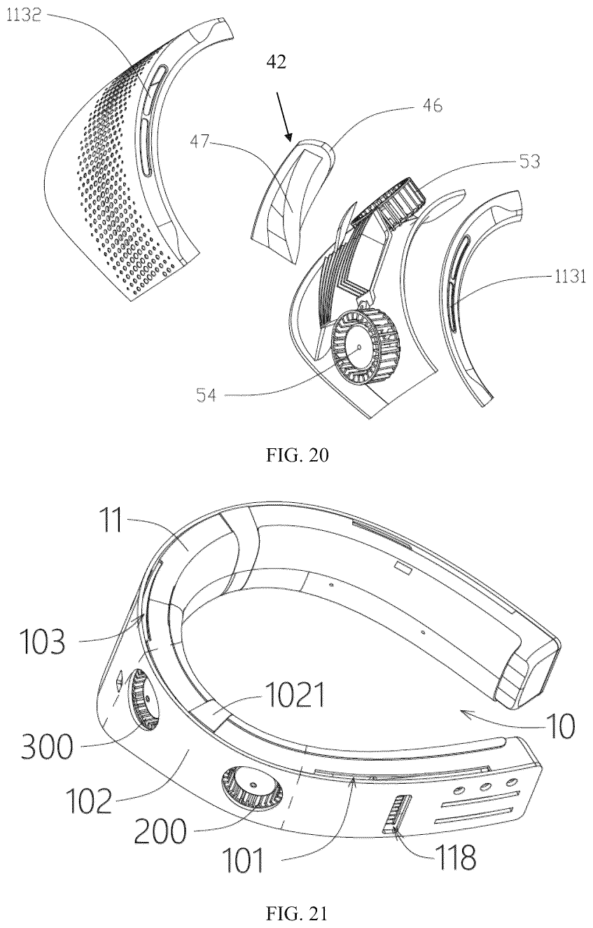

As shown in FIGS. 19 and 20, a fourth embodiment of the application is provided. Compared with the second embodiment, in the instant embodiment, the second air outlet opening 113 comprises a first sub air outlet opening 1131 and a second sub air outlet opening 1132 arranged side by side in the thickness direction of the main body 10. The first sub air outlet opening 1131 is arranged closer to the wearing space 10 than the second sub air outlet opening 1132. The portable temperature regulation device comprises a second air guiding member 42 which is arranged in the interior of the middle portion. The air guide member 42 comprises a first baffle plate 46 and a second baffle plate 47 connected to each other. The first baffle plate 46 and the second baffle plate 47 are integrally formed, and the second baffle plate 47 and the first baffle plate 46 are arranged at an angle. The second baffle plate 47 is connected to a portion of the first baffle plate 46 located between two sides of the first baffle plate 46. The second baffle plate 47 is arranged across two ends of the first baffle plate 46. The first baffle plate 46 and the inside wall of the middle portion form a second air channel 310, and the second baffle plate 47 is located in the second air channel 310 and divides the second air channel 310 into a third sub air channel and a fourth sub air channel. The third sub air channel is in communication with the first sub air outlet opening 1131, and the fourth sub air channel is in communication with the second sub air outlet opening 1132. The third sub air channel and the fourth sub air channel do not interference with each other when guiding airflows such that the volume of air exiting the first sub air outlet opening 1131 and the second sub air outlet opening 1132 is relatively uniform. The structure of other components of this embodiment is the same as that of the above-mentioned the second embodiment.

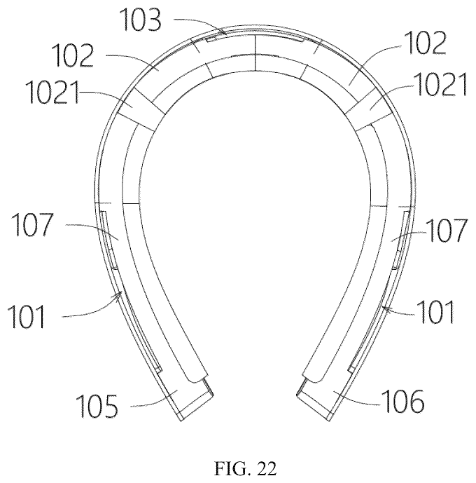

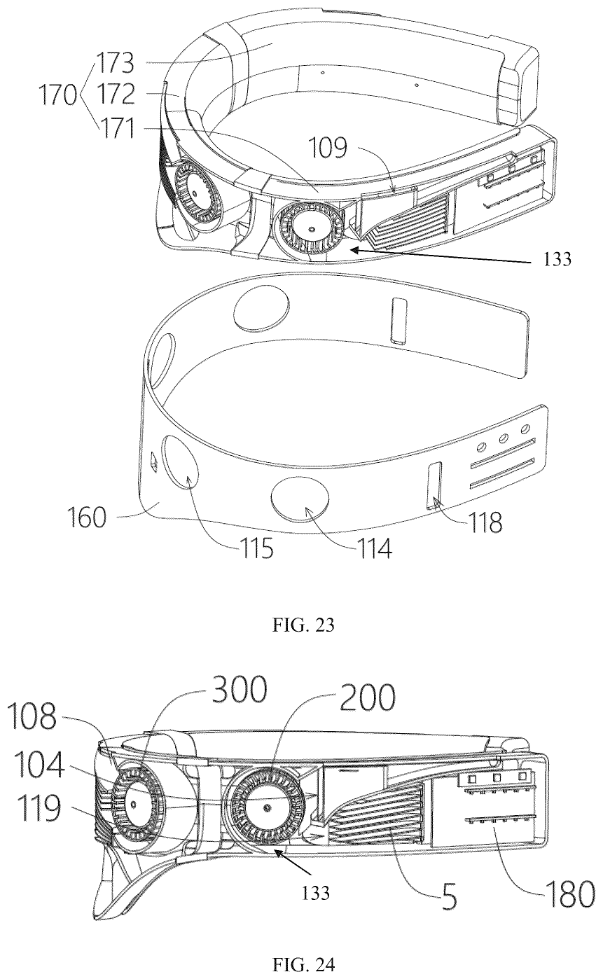

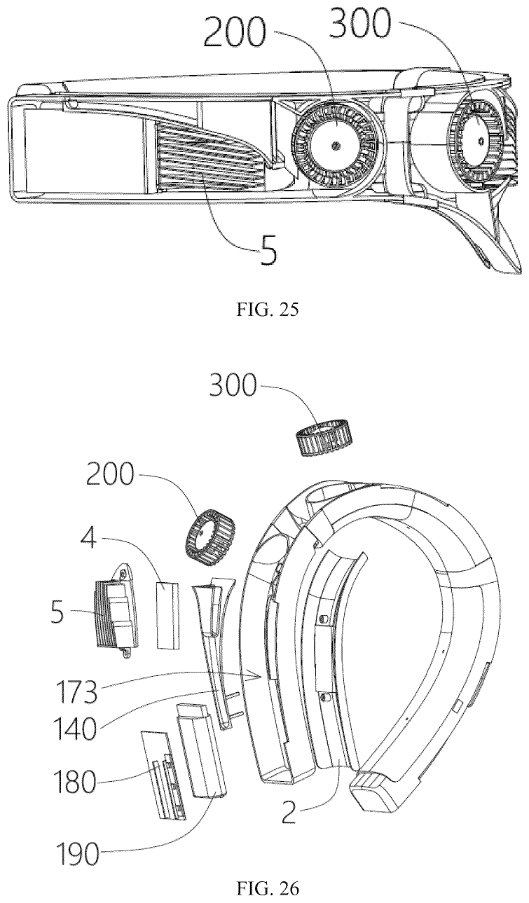

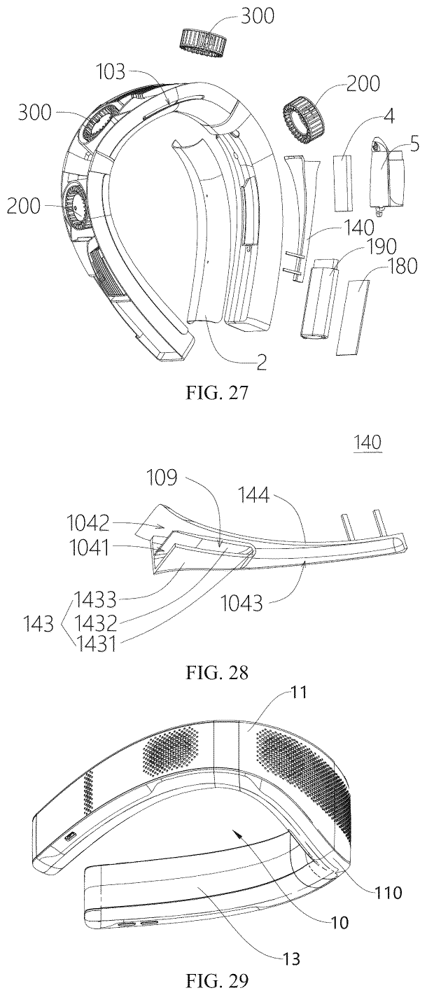

Referring to FIGS. 21-28, a portable temperature regulation device according to a fifth embodiment of the present application comprises a main body 11 which comprises two external housings 107 that are connected. Each of the external housings 107 is provided, in the interior thereof, with a first fan 200 and a second fan 300. The external housing 107 is provided, in sequence along a length direction, with a first air outlet opening 101, an avoidance portion 102, and a second air outlet opening 103. The first fan 200 and the second fan 300 are arranged between the first air outlet opening 101 and the second air outlet opening 103, and the first fan 200 and the second fan 300 are located in the avoidance portion 102. The first fan 200 is configured to supply air to the first air outlet opening 101, and the second fan 300 is configured to supply air to the second air outlet opening 103.

In the instant embodiment, the main body 11 is curved to enclose a wear space 10. An opening in communication with the wear space 10 is formed between two ends of the main body 11. The main body 11 can be formed by joining the two external housing 107, or it is feasible to artificially divide it into the two external housing 107.

Further, the main body 11 has a first end 105 and a second end 106 in a length direction thereof. The length from the avoidance portion 102 which is closer to the first end 105 than the second end 106, to the first end 105 is less than the length from the avoidance portion 102 to the second end 106, and the length from the avoidance portion 102 to the first end 105 is greater than the length from the avoidance portion 102 to the middle of the main body 11.

In the instant embodiment, the avoidance portion 102 is arranged to correspond to one ear of the user. The first end 105 is located at one of the external housings 107, and the second end 106 is located at the other one of the external housings 107. The second air outlet opening 103 is arranged in the middle of the main body 11. Since the avoidance portion 102 is arranged at one of the external housings 107, the length from the avoidance portion 102 arranged at one of the external housings 107 to the first end 105 is less than the length from the same avoidance portion 102 to the second end 106 located at the other one of the external housings 107. Further, since the position of the ear is closer to the back of the head relative to the face of the user, the length from the avoidance portion 102 to the first end 105 being greater than the length from the avoidance portion 102 to the middle of the main body 11 allows the avoidance portion 102 to be located exactly below the ear when the user wears the portable temperature regulation device.

In one embodiment, the external housing 107 is provided, in the interior thereof, with a first air outlet channel 104 and a second air outlet channel 108. The first air outlet channel 104 is located at one side of the first fan 200 away from the middle of the avoidance portion 102. One end of the first air outlet channel 104 is in communication with an exit opening of the first fan 200, where the first fan 200 supplies air out through the exit opening, and another end is extended in a direction away from the first fan 200 and is in communication with the first air outlet opening 101. The second air outlet channel 108 is located at one side of the second fan 300 away from the avoidance portion 102. One end of the second air outlet channel 108 is in communication with an exit opening of the second fan 300, and another end is extended in a direction away from the second fan 300 and is in communication with the second air outlet opening 103.

It should be noted that, in this embodiment, the middle of the avoidance portion 102 refers to the portion of the avoidance portion 102 located between the first fan 200 and the second fan 300.

The cross-sectional area of the first air outlet channel 104 gradually decreases from one end thereof close to the first fan 200 toward another end away from the first fan 200. Since the air volume in the first air outlet channel 104 gradually decreases as being blowing out from the first air outlet opening 101, the air outgoing speed at the end of the first air outlet opening 101 close to the first fan 200 is greater than the air outgoing speed at the end far from the first fan 200. By reducing the cross-sectional area of the first air outlet channel 104, the air outgoing speed at the end of the first air outlet opening 101 away from the first fan 200 can be increased, making the overall air outgoing of the first air outlet opening 101 more uniform.

Further, the first air outlet channel 104 comprises a first sub air channel 1041. One end of the first sub air channel 1041 close to the first air outlet opening 101 is provided with a third air outlet opening 109 in communication with the first air outlet opening 101. The length direction of the third air outlet opening 109 is the same as the length direction of the first air outlet opening 101.

The external housing 107 is further provided, in the interior thereof, with a flow directing member 140. The flow directing member 140 is located on one side of the first fan 200 away from the middle of the avoidance portion 102. The first air outlet channel 104 is at least partly arranged in the flow directing member 140. The flow directing member 140 comprises a flow directing plate 143 which is partially curved to surround and form the first sub air channel 1041.

Further, the flow directing member 140 comprises a base 144 and the flow directing plate 143 is arranged at one end of the base 144. The flow directing plate 143 comprises a curved portion 1431 and a first flow directing portion 1432 and a second flow directing portion 1433 arranged on the same side of the curved portion 1431. The first flow directing portion 1432 and the second flow directing portion 1433 are extended in the length direction of the base 144.

Further, the first air outlet channel 104 further comprises a second sub air channel 1042 and a third sub air channel 1043. The first sub air channel 1041 and the second sub air channel 1042 are arranged side by side at the same end of the third sub air channel 1043, and the second sub air channel 1042 is connected to the third sub air channel 1043.

Specifically, the extension direction of the first sub air channel 1041 is substantially the same as the extension direction of the second sub air channel 1042 and the extension direction of the third sub air channel 1043. The width of the first sub air channel 1041 and the width of the second sub air channel 1042 are both smaller than the width of the third sub air channel 1043.

In one embodiment, the portable temperature regulation device further comprises a temperature conducting member 2 arranged on the external housing 107. The temperature conducting member 2 is at least partially arranged on the avoidance portion 102. The first air outlet opening 101 and the second air outlet opening 103 cannot directly adjust the temperature of the part of the user facing the avoidance portion 102. The arrangement of the temperature conducting member 2 realizes contact type temperature adjustment of the part of the user corresponding to the avoidance portion 102.

Further, the external housing 107 is further provided, in the interior thereof, with a temperature regulation member 4 and a heat dissipating member 5. The temperature conducting member 2 and the heat dissipating member 5 are respectively arranged on opposite sides of the temperature regulation member 4 and is in thermal conduction connection with the temperature regulation member 4.

In the instant embodiment, the temperature regulation member 4 is a semiconductor cooler device, and the temperature conducting member 2 is made of aluminum alloy. The temperature conducting member 2 is arranged on one side of the inside casing 170 of the external housing 107 close to the wear space 10. The temperature conducting member 2 is configured to evenly transfer the cold or heat generated by the semiconductor cooler device to the neck of the user to achieve the function of cooling or heating. The heat dissipating member 5 comprises a plurality of heat dissipation fins for increasing the contact area between the airflow blown by the first fan 200 and the heat dissipating member 5 to improve the heat dissipation efficiency.

In one embodiment, the external housing 107 is further provided with a heat dissipation opening 118. The heat dissipation opening 118 is located at one side of the first fan 200 away from the middle of the avoidance portion 102. The external housing 107 is provided, in the interior thereof, with an air channel 119. The air channel 119 is located at one side of the first fan 200 away from the second fan 300. One end of the air channel 119 is in communication with an exit opening of the first fan 200, and another end is extended in a direction away from the first fan 200 and is in communication with the heat dissipation opening 118. The heat dissipating member 5 is accommodated in the air channel 119.

Specifically, the outside casing 160 of the external housing 107 is provided with a first air inlet opening 114, a second air inlet opening 115, and the heat dissipation opening 118. The first air inlet opening 114 is arranged to face to the air intake side of the first fan 200. The second air inlet opening 115 is arranged to face to the air intake side of the second fan 300. The first air outlet opening 101 is arranged at a junction of the outside casing 160 and the inside casing 170. The external housing 107 is provided, in the interior thereof, with a receiving compartment 133 in communication with the first air outlet opening 101. The first fan 200 is arranged in the receiving compartment 133. The receiving compartment 133 is further provided with a flow directing member 140 arranged in the interior thereof. The first fan 200 is located between the flow directing member 140 and the second fan 300. The flow directing member 140 separates a portion of the receiving compartment 133 to form an air channel 119.

Further, the receiving compartment 133 is also provided, in the interior thereof, with a control circuit board 180 and a power supply 190. The control circuit board 180 is in electrical connection with the power supply 190, the temperature regulation member 4, the first fan 200, and the second fan 300 respectively. The control circuit board 180 is configured to control, according to an instruction inputted by the user, activation/deactivation positions of each fan and operation mode (cooling or heating) and operation temperature of the temperature regulation member 4.

In this embodiment, the main body 11 comprises an upper side and a lower side that are opposite to each other in the height direction thereof. Two first fans 200 and two second fans 300 are mounted in the main body 11. Two second air outlet openings 103 are both located on the upper side of the main body 11, or the two second air outlet opening 103 are both located on the lower side of the main body 11, or one of the second air outlet openings 103 is located on the upper side of the main body 11, while another one of the second air outlet openings 103 is located on the lower side of the main body 11.

In the instant embodiment, the two first air outlet openings 101 are both arranged on the upper side of the main body 11, and one of the second air outlet openings 103 is arranged on the upper side of the main body 11, while another one of the second air outlet openings 103 is located on the lower side of the main body 11. When the user wears the portable temperature regulation device on the neck, the upper part of the main body 11 faces the user's head in order to blow air to the face and the back of the neck of the user. The lower part of the main body 11 faces the user's body in order to blow air to the back of the user for cooling. The avoidance portions 102 correspond to the user's ears in order to prevent the airflow exiting from the first air outlet opening 101 and the second air outlet opening 103 from directly blowing toward the ears, thereby reducing the noise heard by the user.

In one embodiment, the avoidance portion 102 is provided with a flexible connecting member 1021.

Specifically, the main body 11 comprises the outside casing 160 and the inside casing 170 connected to each other, and the inside casing 170 is closer to the wear space 10 than the outside casing 160. The outside casing 160 is a whole elongated strip-like structure. The inside casing 170 comprises a first casing segment 171, a second casing segment 172, and a third casing segment 173 arranged in sequence along the length direction of the outside casing 160. A flexible connecting member 1021 is arranged between the first casing segment 171 and the second casing segment 172, and another flexible connecting member 1021 is arranged between the second casing segment 172 and the third casing segment 173. The flexible connecting members 1021 can be soft rubber. The arrangement of the flexible connecting members 1021 helps improve the capability of deformation of the main body 11 for easing the adjustment of the size of the wear space 10 to suit for wearing by various users.

The portable temperature regulation device of this embodiment comprises the main body 11 which comprises two parts each of which is provided, in the interior thereof, with the first fan 200 and the second fan 300. The external housing 107 of each part is provided, in sequence along the length direction, with the first air outlet opening 101, the avoidance portion 102, and the second air outlet opening 103. The first fan 200 and the second fan 300 are arranged between the first air outlet opening 101 and the second air outlet opening 103, and the first fan 200 and the second fan 300 are located in the avoidance portion 102. When the portable temperature regulation device is worn on the user's neck, the first fan 200 generates an airflow to move in a direction away from the middle of the avoidance portion 102 and blow out from the first air outlet opening 101, and the second fan 300 generates an airflow to move in a direction away from the middle of the avoidance portion 102 and blow out from the second air outlet opening 103. The avoidance portion 102 corresponds, in position, to the user's ear, so that on the one hand, air exiting directly toward the user's ear from the first air outlet opening 101 and the second air outlet opening 103 is avoided to thereby reduce the nose received by the ear, and on the other hand, the airflows generated by the first fan 200 and the second fan 300 are blown out in directions away from the avoidance portion 102 to thereby make the noise not easily entering the user's ear so as to further reduce the nose received by the ears to improve the use experience of the user.

It should be noted that during the course when the airflow generated by the fan passes through the air channel to flow toward the air outlet opening, the airflow and a wall of the air channel constantly rub against each other and generate noise, and the noise is superimposed along the moving direction of the airflow and reaches the maximum at the air outlet opening. When the portable temperature regulation device is worn on the user's neck, since the avoidance portion 102 is the area on the main body 11 that is closest to the user's ear, by arranging the first fan 200 and the second fan 300 at two opposite sides of the avoidance portion 102 and to discharge air in directions away from the middle of the avoidance portion 102, the noise is superimposed along the direction away from the user's ear, thereby reducing the noise received by the ear.

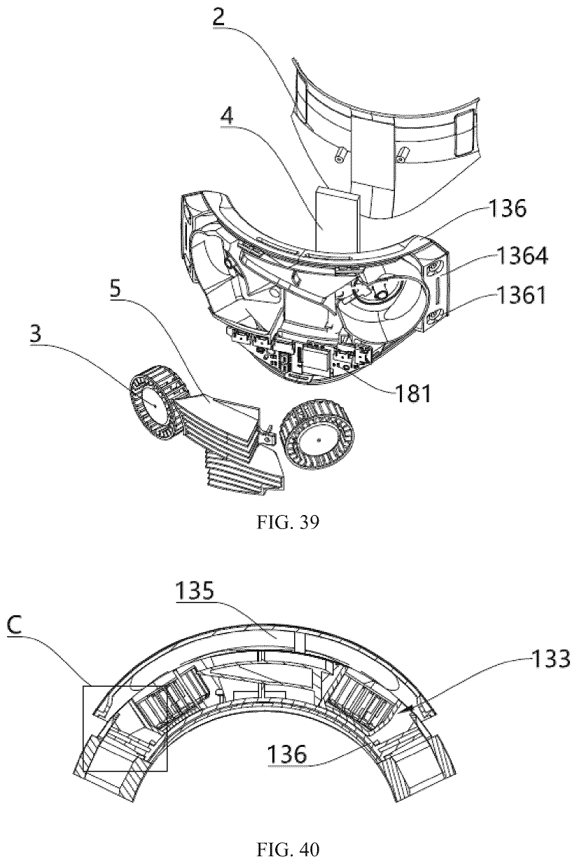

Referring to FIGS. 29-43, a portable temperature regulation device according to a sixth embodiment of the application comprises a main body 11 and a covering member 60. The components that fulfill the functions of temperature regulation, such as blowing and contact cooling, are mainly arranged the main body 11. For example, the main body 11 is provided with an air inlet opening 15, an air outlet opening 16, and a heat dissipation opening 14, and an air channel 17 connecting the air inlet opening 15 and the heat dissipation opening 14. The main body 11 is provided, in the interior thereof, with a fan 3, a heat dissipating member 5, and a temperature regulation member 4 (see FIGS. 33 and 39) for fulfilling the temperature regulation function of the main body 11. The fan 3 is arranged in the air channel 17 and corresponds to the air inlet opening 15. Specifically, the air inlet opening 15 faces the fan 3 and the center of the air inlet opening 14 is aligned with the axis of the fan 3. The heat dissipating member 5 is arranged in the air channel 17 and corresponds to the heat dissipation opening 14. The temperature regulation member 4 is arranged outside the air channel 17 and is thermal conduction connection with the heat dissipating member 5. The temperature regulation member 4 is for example a semiconductor cooling sheet, which fulfills the function of contact cooling in combination with a temperature conducting member 2 connected to the side of the temperature regulation member 4 opposite to the heat dissipating member 5 (see FIGS. 33 and 39). The covering member 60 is connected to the main body 11 and covers the air inlet opening 15 and the heat dissipation opening 14. The covering member 60 is provided with a first through aperture array 601 that is in communication with the air inlet opening 15 and a second through aperture array 602 that is in communication with the heat dissipation opening 14.

In the instant embodiment, the housing/shell material thickness of the main body 11 can be set to be relatively thick, having a predetermined strength for better protection of components arranged therein. To reduce the difficulty of producing holes in the shell of the main body 11, the air inlet opening 15 can be formed as an entire large hole close in diameter to the fan 3. Similarly, the heat dissipation opening 14 can be formed as a relatively large hole. The first through aperture array 601 and the second through aperture array 602 both include a plurality of apertures. The size of the apertures of the first through aperture array 601 is much less than the size of the air inlet opening 15, and the size of the apertures of the second through aperture array 602 is much less than the size of the heat dissipation opening 14. In this way, forming a relatively large hole in the main body 11 is less difficult than directly forming multiple small apertures in the main body 11. For forming the small apertures in the covering member 60, the thickness of the covering member 60 can be selected to be relatively small, not affected by the material thickness of the main body 11, and the difficulty of producing holes being small. Further, the first through aperture array 601 and the second through aperture array 602 can also prevent the entry of foreign objects such as hair. Further, the first through aperture array 601 and the second through aperture array 602 are separately formed in the covering member 60, and in the instant embodiment, the main body 11 can be a curved structure. When a mounting structure for the fan 3, the heat dissipating member 5, and the temperature regulation member 4 is set up in the interior, the operation of producing apertures in the covering member 60 is not affected by the complicated structure in the interior of the main body 11, and the operation of producing apertures is convenient. Further, the covering member 60 can choose a material, color, and pattern different from the main body 11 to realize diversified selection designs of the portable temperature regulation device.

Specifically, the portable temperature regulation device provided in the instant embodiment will be described in detail below in conjunction with the neck-mounted structure shown in FIG. 29.

FIG. 29 illustrates a neck-mounted portable temperature regulation device. As shown in FIG. 29, the portable temperature regulation device exhibits a U-shaped structure as a whole, comprising a base portion 110 and two arm portions 13 arranged at two opposite ends of the base portion 110. The two arm portions 13 both extend toward one side of the base portion 110 and enclose and form the wear space 10 in combination with the base portion 110. When worn, the human neck is located in the wear space 10. Of course, the portable temperature regulation device can also be handheld, neck mounted, and waist belt fashion, and the instant embodiment is not limited thereto.

In the portable temperature regulation device shown in FIG. 29, the base portion 110 and the two arm portions 13 can both be provided with independent components having the temperature regulation functions (namely the fan 3, the heat dissipating member 5, and the temperature regulation member 4). In the instant embodiment, it can be regarded that the main body 11 is the portion that includes the components of temperature regulation function, or alternatively, it can be regarded as any one of the base portion 110 and the two arm portions 13 independently including the structure of the main body 11. In one embodiment, the base portion 110 and the arm portions 13 both include the structure of the main body 11, and it can also be regarded as the main body 11 comprising the base portion 110 and the arm portions 13, and the base portion 110 and the arm portions 13 are respectively provided, on one side thereof opposite to the wear space 10, with the air inlet openings 15 and the heat dissipation openings 14 and correspondingly connected with the covering member 60. That is, the base portion 110 is connected to the covering member 60, and the arm portions 13 are also connected to the covering member 60.

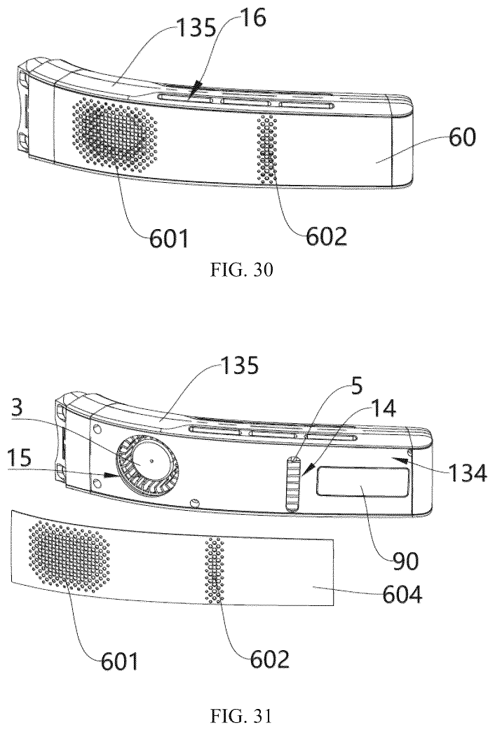

FIGS. 30-33 show the structure of one arm portion 13 of the portable temperature regulation device.

Referring to FIG. 30, the portion of the portable temperature regulation device comprises the arm portion 13 and the covering member 60. Based on the direction illustrated in FIG. 30, the aperture array that is on the left side of the covering member 60 and similar to a circular array is the first through aperture array 601, while the aperture array on the right side and similar to an elongated strip array is the second through aperture array 602. With reference to the exploded view of FIG. 31, the main body 11 is formed with a mounting trough 134, and the covering member 60 can be mounted in the mounting trough 134 to form the structure as shown in FIG. 30. In this embodiment, the covering member 60 can be fixed in the mounting trough 134 with glue and can also be fixed in the mounting trough 134 by other means, such as snap-on connection. Based on the direction shown in FIG. 31, the circular opening in the left side of the main body 11 is the air inlet opening 15, and the elongated opening in the right side is the heat dissipation opening 14. When the covering member 60 is mounted on the main body 11, the first through aperture array 601 is aligned with the air inlet opening 15, and the second through aperture array 602 is aligned with the heat dissipation opening 14, so that airflow outside the main body 11 can enter the main body 11 from the first through aperture array 601 through the air inlet opening 15. The airflow inside the main body 11 can be blown out from the second through aperture array 602 through the heat dissipation opening 14.

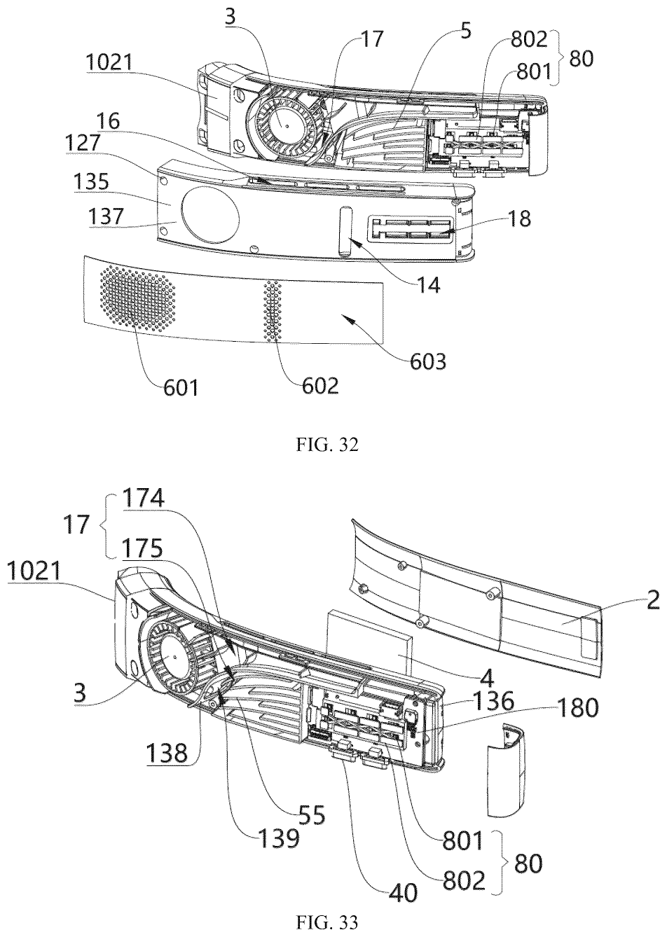

Referring to FIG. 32, the housing/shell of the main body 11 of this portion (namely the arm portions 13) can be divided into an outside casing 135 and an inside casing 136 that are opposite to each other. The outside casing 135 and the inside casing 136 are connected and assembled by screws, for example. The outside casing 135 and the inside casing 136 form therebetween a receiving compartment 133 in which the fan 3, the heat dissipating member 5, and the temperature regulation member 4 are received and also enclose and form an air channel 17. The outside casing 135 comprises an external plate 137, and the air inlet opening 15 and the heat dissipation opening 14 are arranged on the external plate 137. The thickness of the covering member 60 is less than the thickness of the external plate 137. The thickness of the covering member 60 is made smaller, so that the depth of the apertures of the first through aperture array 601 and the second through aperture array 602 is also smaller, which facilitates to improve the air flowing effect.

In the neck-mounted structure shown in FIG. 29, the outside casing 135 is located at one side of the inside casing 136 opposite to the wear space 10, so that the air inlet opening 15 and the heat dissipation opening 14 are located at the side of the main body 11 opposite to the wear space 10.

The fan 3 and the heat dissipating member 5, as depicted in FIG. 33, are arranged in the interior of the inside casing 136. A partition member 138 is arranged in the interior of the inside casing 136 to divide the air exiting side of the fan 3 into an air egress channel 174 and a heat dissipation air channel 175. The partition member 138 defines an air passage opening 139, and a part of the airflow generated by the fan 3 passes through the air passage opening 139 to enter into the air channel 175 to contact and exchange heat with the heat dissipating member 5 and finally blow out from the heat dissipation opening 14. The sides of the outside casing 135 and the inside casing 136 that are adjacent to the air inlet opening 15 are formed with air outlet openings 16, and another part of the airflow generated by the fan 3 passes through the air egress channel 174 to blow out from the air outlet openings 16, fulfilling an effect of lowering temperature by blown airflow. The heat dissipating member 5 comprises a plurality of heat dissipating fins 55, and heat dissipating gaps are formed between adjacent heat dissipating fins 55. As shown in FIG. 33, the plurality of heat dissipating fins 55 are extended in a direction (left-right direction/length direction of the main body) from the fan 3 to the heat dissipation opening 14. The length direction of the heat dissipation opening 14 is perpendicular to the extension direction of the plurality of heat dissipating fins 55, that is, the heat dissipation opening 14 extends in an up-down direction (the height direction of the main body), allowing the plurality of heat dissipating gaps to dissipate heat independently.

In one embodiment, continuing to refer to FIG. 32, a display module 80 is arranged in the interior of the main body 11. The main body 11 is formed with a display window 18 at a location corresponding to the display module 80. The covering member 60 has a light-transmitting region 603. The covering member 60 covers the display window 18 with the light-transmitting zone 603 being arranged to correspond to the display window 18. In the instant embodiment, the light-transmitting region 603 refers to an area of the covering member 60 through which the display module 80 is visible. In fact, the covering member 60 can be entirely made of a light-transmitting plate, or can be a plate of which only the light-transmitting zone 603 transmits light. A screen display function can be fulfilled with the covering member 60.

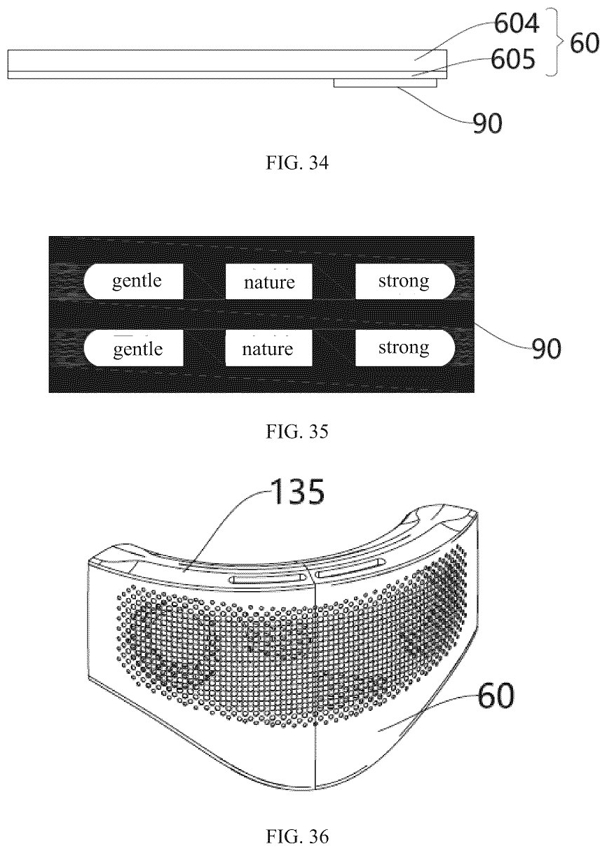

In some embodiments, referring to FIG. 34, the covering member 60 may comprise a substrate 604 and a thin film layer 605 arranged on the side of the substrate 604 close to the main body 11. The substrate 604 is for example made of a transparent PC (polycarbonate) material, and the thin film layer 605 may be a PMMA (polymethyl methacrylate) coating layer and is printed on the substrate 604 by silk screen printing, so as to achieve a light-transmitting but non-see-through effect. When the covering member 60 covers the main body 11, the portion other than the light-transmitting region 603 corresponding to the display module 80 fulfills covering of the screw holes 127 on the main body 11 (see FIG. 32), achieving a more aesthetically pleasing effect. The light-transmitting region 603 is arranged to correspond to the display module 80, so that when the display module 80 does not emit light for displaying, the display module 80 that is covered by the covering member 60 cannot be seen through the light-transmitting region 603. When the display module 80 emits light for displaying, the light transmits through the light-transmitting region 603 to achieve an effect of displaying.

The display module 80 can be an LCD display screen or a combination of multiple indicator lights. In some embodiments, the display module 80 comprises a light-emitting element 801 and a light guide structure 802 arranged at one side of the light-emitting element 801 close to the covering member 60. The light guide structure 802 abuts against the light-transmitting zone 603 of the covering member 60. The light-emitting element 801 can be for example LED beads of the same or different colors. The light guide structure 802 can be for example transparent PC material to direct light emitting from the light beads to the covering member 60. A specific light guide structure 802 may comprise a plurality of light guide columns corresponding to a plurality of light beads one by one, so that the light emitting from each light bead does not interfere with each other. In the instant embodiment, for example, referring to FIG. 33, a control circuit board 180 is further provided in the interior of the inside casing 136, the light-emitting element 801 is fixed on the control circuit board 180 and in electrical connection with the control circuit board 180. The inside casing 136 is also provided with a pushbutton switch 40 electrically connected to the control circuit board 180. The control circuit board 180 is electrically connected to the fan 3 and the temperature regulation member 4. In use, the pushbutton switch 40 can be operated to fulfill ON/OFF switching and function selection and adjustment for the portable temperature regulation device and controls the fan 3 and the temperature regulation member 4. When different functions are selected and adjusted, the control circuit board 180 controls different light-emitting elements 801 to emit light to fulfill displaying of the corresponding function.

In some embodiments, the portable temperature regulation device further comprises a light diffusion plate 90. The light diffusion plate 90 covers the display window 18 and is located between the light-transmitting zone 603 and the main body 11. The light diffusion plate 90 has a pattern marking. The light diffusion plate 90 can be made of PET (polyethylene terephthalate) with diffuser powder. For example, referring to FIG. 35, which is a drawing of an embodiment of the light diffusion plate 90, the black area in FIG. 35 is the material part of the light diffusion plate 90, and the white area is the hollowed part of the light diffusion plate 90. When a light-emitting element 801 at a location corresponding to the display module 80 emits light, the light passes through the corresponding hollowed part to fulfill an effect of displaying, while the hollowed parts that are not illuminated by the light do not display according to the light-transmitting but non-see-through principle of the covering member 60, thereby achieving the displaying effect while ensuring aesthetics. The pattern marking on the light diffusion plate 90 can be text or graphics, and this embodiment is not limited thereto.

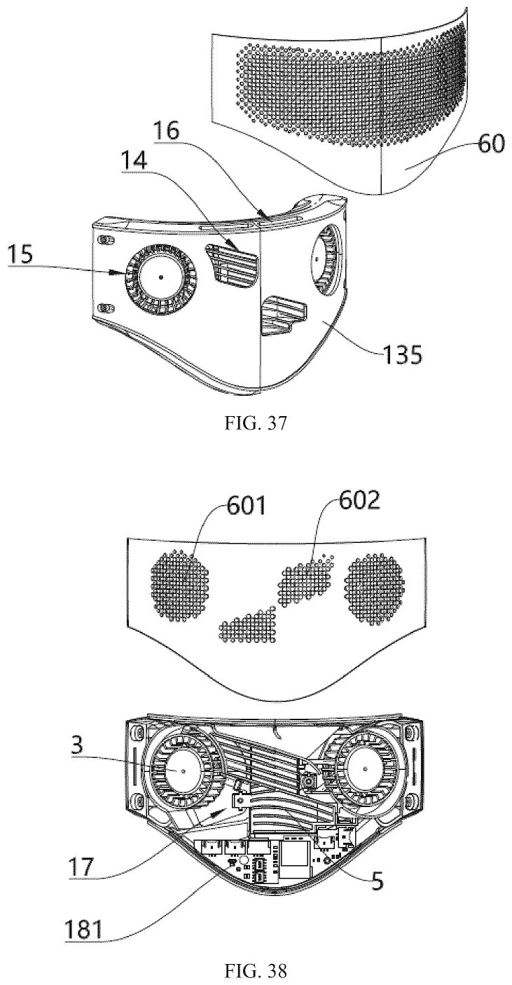

FIGS. 36-39 shows the structure of the base portion 110 of the portable temperature regulation device. As shown in FIG. 36, the portion of the portable temperature regulation device includes the base portion 110 and the covering member 60. According to the exploded view shown in FIG. 37, the internal structure of the side of the main body 11 close to the covering member 60 and the structure of the covering member 60 close to the main body 11 are shown. The main body 11 is provided with two air inlet openings 15 and two heat dissipation openings 14. The two heat dissipation openings 14 are located between the two air inlet openings 15. Referring to FIG. 38, a fan 3 is provided at each position corresponding to the two air inlet openings 15, and the heat dissipating member 5 is arranged between the two fans 3. Referring to the direction illustrated in FIG. 38, the heat dissipating member 5 comprises two parts. Each part has a plurality of heat dissipating fins with gaps formed therebetween. Inlets of the gaps of the two parts are arranged opposite to each other. The gaps of the part located at the upper side is in communication with the air exiting side of the fan 3 on the right side, and the gaps of the part located at the lower side is in communication with the air exiting side of the fan 3 on the left side. Correspondingly, the number of the first through aperture arrays 601 provided on the covering member 60 is also two, which are arranged to respectively correspond to the two air inlet openings 15. The second through aperture arrays 602 are arranged between the two first through aperture arrays 601 to correspond to the heat dissipation openings 14. It should be noted that, as shown in FIGS. 36 and 37, a plurality of apertures is formed in the outer side surface of the covering member 60 (the side opposite to the main body 11), wherein some apertures other than the first through aperture arrays 601 and the second arrays 202 are blinded apertures rather than through apertures.

Referring to FIG. 38, the inside casing 136 of the base portion 110 is further provided, in the interior thereof, with for example a connection circuit board 181, and the two fans 3 and the temperature regulation member 4 in the interior of the base portion 110 can be electrically connected by conductive wires to the connection circuit board 181. The connection circuit board 181 is electrically connected by conductive wires to the control circuit board 180, so that the fan 3 and the temperature regulation member 4 in the base portion 110 and the arm portions 13 can be separately controlled to operate by means of the pushbutton switch 40 on the arm portions 13. In this embodiment, ends of the inside casing 136 and the outside casing 135 of the base portion 110 away from the upper side of the base portion 110 protrude downwardly to form a protrusion portion similar to the protrusion portion 12 of the first embodiment. The connection circuit board 181 is mounted in the protrusion portion 12.

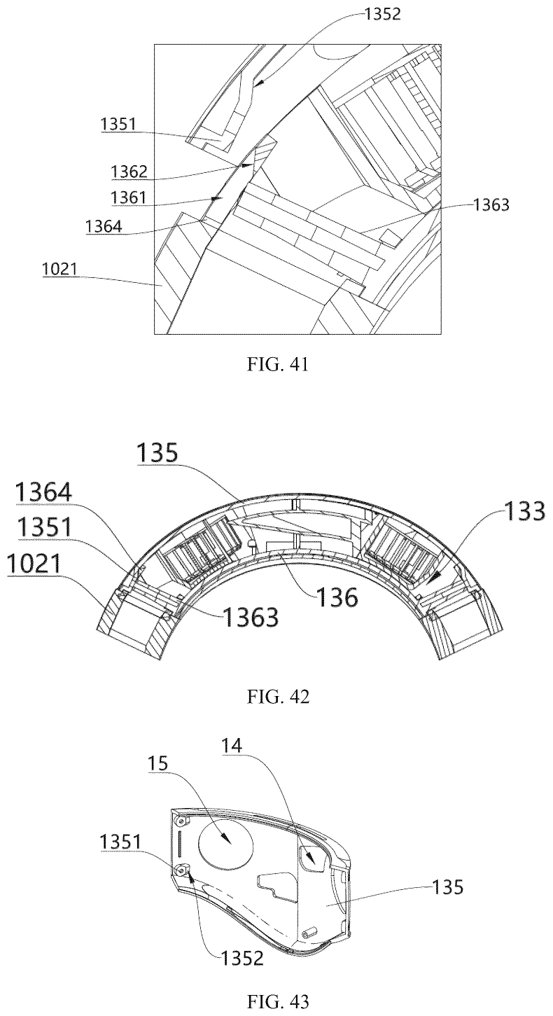

In some embodiments, the inside casing 136 and the outside casing 135 are both curved in a direction from the outside casing 135 to the inside casing 136. Referring to FIG. 40, for example, taking the direction of the base portion 110 as an example, the inside casing 136 and the outside casing 135 are both curved downward to form an arcuate shape. End portions of the inside casing 136 are formed with positioning notches 1361. Referring also to FIGS. 38 and 39, the positioning notches 1361 are arranged at the left and right end sides of the inside casing 136, two at each end side, and a total of four positioning notches 1361. More specifically, referring to the partial enlarged view of area A shown in FIG. 41, one side of the positioning notch 1361 close to the middle of the inside casing 136 forms an inclined notch face 1362 which is inclined in the direction from the end portion to the middle of the inside casing 136. Referring to FIG. 43, one side of the outside casing 135 close to the inside casing 136 is provided with positioning members 1351. The positioning members 1351 are coupled with the positioning notches 1361. More specifically, the positioning member 1351 is provided with a positioning inclined face 1352 that matches the inclined notch face 1362. The inside casing 136 is further provided with a fixing post 1363. The positioning members 1351 extend through the corresponding positioning notches 1361 to be fixed to the corresponding fixing posts 1363 by screws so as to fix the inside casing 136 and the outside casing 135 together as shown in FIG. 42. According to the direction illustrated in FIG. 40, when it needs to mount the outside casing 135 downward onto the inside casing 136, due to the curvature being relatively large, the arrangement of the inclined notch face 1362 helps prevent mutual interference between the outside casing 135 and the inside casing 136 during assembling. Specifically, referring also to the exploded view shown in FIG. 39, the inside casing 136 can be further provided, for example, with a connecting member 1364. The connecting member 1364 can be a nylon member for fulfilling connection between the base portion 110 and the arm portion 13. The positioning notch 1361 may be specifically arranged on the connecting member 1364. Further, similarly, the inside casing 136 of the arm portion 13 may also be provided with a connecting member 1364. Further, a flexible connecting member 1021 may be further provided for example between the base portion 110 and the arm portion 13, and the flexible connecting member 1021 may be made of a silicone rubber material and is sleeved on the connecting member 1364 of the base portion 110 and the connecting member 1364 of the arm portion 13 to fulfill connection between the arm portion 13 and the base portion 110 and to allow angle adjustment between the base portion 110 and the arm portion 13 so as to facilitate wearing.

Similar to description made above for the arm portion 13 being the main body 11, when the base portion 110 is made as the main body 11, the structure, such as the mounting trough 134 and the display module 80, are also provided, and the instant embodiment is not limited thereto.

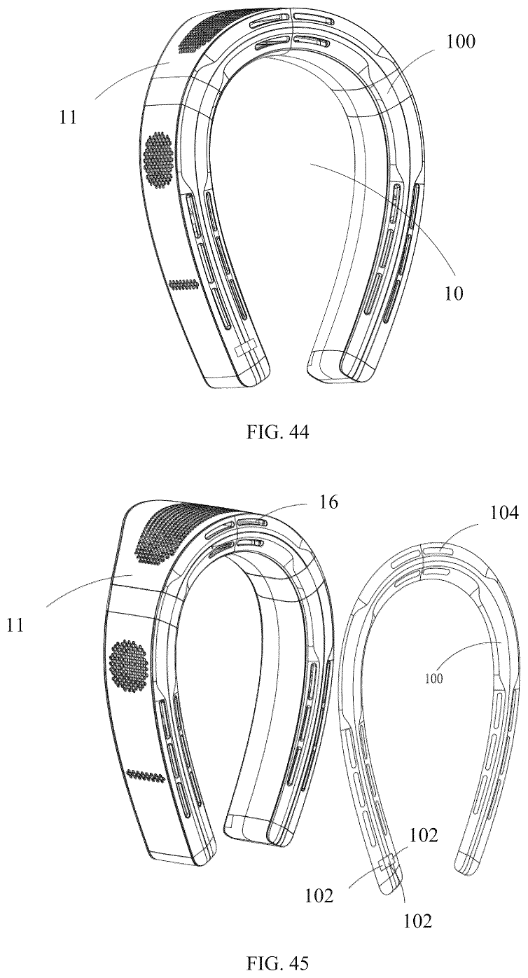

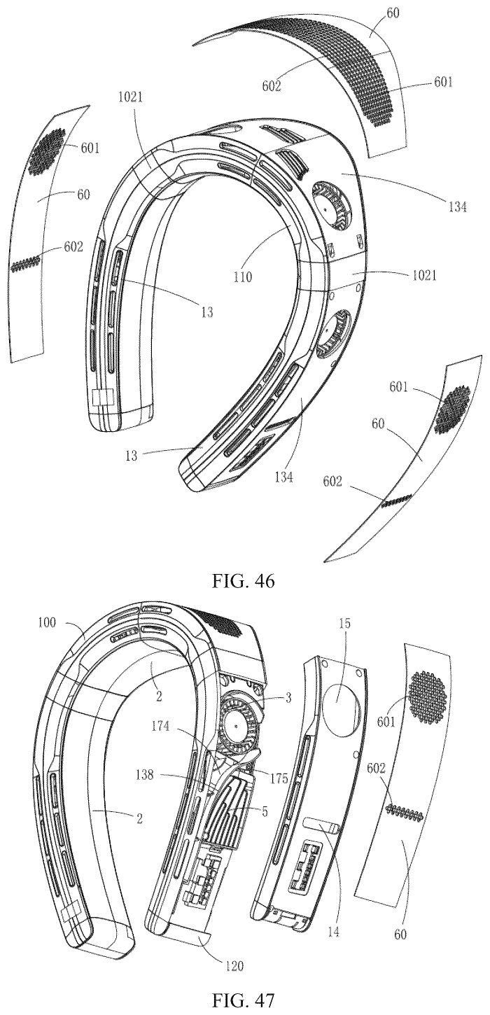

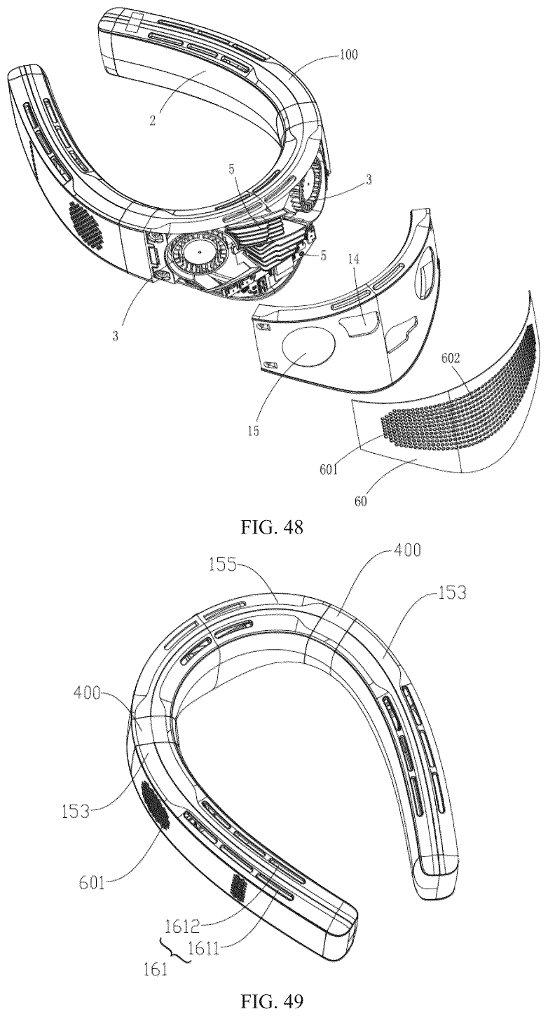

Referring jointly to FIGS. 44-48, a seventh embodiment of the application provides a portable temperature regulation device, which is wearable on a human body, such as the human neck, to perform temperature regulation on the human body, such as blowing air, cooling or heating. Compared with the sixth embodiment, the portable temperature regulation device of the instant embodiment comprises a main body 11, a temperature regulation assembly arranged in the interior of the main body 11, and a color-changing body arranged on the main body 11. The color-changing body, when irradiated by sunlight, changes to different colors according to the different intensities of ultraviolet rays in the sunlight, so as to detect and identify the intensity of ultraviolet rays in the sunlight through color changes, and using the color change effect of the color-changing body to identify the intensity of ultraviolet rays in the sunlight facilitate the user to determine whether protective measures need to be taken. Further, the color-changing body changing color when the portable temperature regulation device is in the sunlight environment brings a cool effect, increases fashion, fun and sense of technology, and enhances user experience.

In the above, the color-changing body may change color in many ways. For example, when the ultraviolet light is stronger, the color of the color-changing body becomes darker, namely changing to different colors of different shades, such as from light orange to dark orange; or when the ultraviolet light is stronger, the color-changing body changes to different colors, such as from gray to red; or when the ultraviolet light reaches a certain intensity, for example, when the ultraviolet light intensity reaches 75 mW/m², the color-changing body will change color, such as from black to red.

In the illustrated embodiment, the color-changing body is implemented as an ultraviolet color change layer 100 arranged on a surface of the main body 11. When irradiated by sunlight, the ultraviolet color change layer 100 changes color according to the intensity of ultraviolet rays. Preferably, the ultraviolet color change layer 100 is arranged on the top surface of the main body 11. For example, the ultraviolet color change layer 100 is designed to match the shape of the main body 11 to cover the entire top surface. The top surface of the main body 11 can be for example an undulating concave-convex structure, and the ultraviolet color change layer 100 is designed to be a matched concave-convex structure to fit perfectly. In this, the ultraviolet color change layer 100 can be an ultraviolet color-changing ink coated on the surface of the main body 11, such as being coated on the surface of the main body 11 by silk-printing. The ultraviolet color change layer 100 can also be a hard or soft cover made of an ultraviolet photosensitive material. The ultraviolet color change layer 100 can be fixed, in a detachable manner, to the top surface of the main body 11 by means of adhesive and snap fitting, so as to allow a user to easily replace the ultraviolet color change layer 100 for failure or being damaged. Further, since the top surface of the main body 11 is the surface that is easily observed by the user, the ultraviolet color change layer 100 is arranged on the top surface, so that the user can timely observe the ultraviolet color change layer 100 changing color to thereby facilitate the user to determine whether protective measures need to be taken.

In some embodiments, the main body 11 is, for example, a plastic material, and the color-changing body is implemented as ultraviolet color change powder, that is, when the ultraviolet color change powder is irradiated by sunlight, according to the intensity of the ultraviolet rays in the sunlight, the ultraviolet color change powder change to different colors. In making the main body 11, the plastic material of the main body 11 and the ultraviolet color change powder are mixed and made into a plastic part (a color-changing main body 11) through injection molding, that is the color-changing body and the main body 11 are integrated as one piece, where the color-changing body is dispersed in the housing material to make a color-changing shell. When the ultraviolet intensity changes, the entire main body 11 undergoes a corresponding color change. It should be pointed out that in other embodiments, it is also possible to have the ultraviolet color change powder and at least a portion of the main body 11 made, by means of the above-described mixing and injection molding process, into a main body 11 having a part that is color changeable, where when the ultraviolet intensity changes, the part of the main body 11 undergoes a corresponding color change locally.

In order to facilitate the user to quickly and accurately identify the ultraviolet intensity corresponding to the color of the color-changing body, in the illustrated embodiment, a color-comparing body is arranged on the main body 11. The color-comparing body is used for color comparison with the color-changing body, and according to the result of comparison, the user can quickly determine whether protective measures need to be taken. In some embodiments, the color-comparing body can be set to just one fixed color, and if the color-changing body changes to such a color, it means that the ultraviolet intensity exceeds a preset standard and protective measures need to be taken; if the color-changing body does not change color, it means that the ultraviolet intensity does not exceed the preset standard and no protective measures need to be taken.

In the instant embodiment, the color-comparing body comprises a plurality of color comparison zones 102 having different fixed colors, that is the color-comparing body comprises a plurality of fixed colors, and the color of each color comparison zone 102 is different from the colors of other color comparison zones 102, and each color corresponds to an ultraviolet intensity. Comparing the color of the color-changing body with the colors of the color comparison zones 102 allows for identifying the current ultraviolet intensity to thereby determine if protective measures need to be taken. The plurality of color comparison zones 102 can be arranged adjacent to each other in sequence, for example, being arranged adjacent to each other in sequence along the width direction, thickness direction or length direction of the main body 11. In the illustrated embodiment, the plurality of color comparison zones 102 are arranged as three, and the three color comparison zones are arranged adjacent to each other in sequence along the width direction of the main body 11.

Optionally, the color-comparing body may be arranged on the main body 11, such as being arranged on the outer surface of the main body 11; or may be alternatively arranged on the ultraviolet color change layer 100 of the main body 11 for being convenient for the user to compare and observe.

In some embodiments, the color-changing body may also comprise a plurality of color-changing zones for detecting different ultraviolet intensities. The user can determine the ultraviolet intensity according to the color change of each color-changing zone, and thus determine whether protective measures need to be taken. For example, three color-changing zones are provided, which are respectively a first color-changing zone, a second color-changing zone, and a third color-changing zone, and the three color-changing zones correspond to different ultraviolet intensities. When only the first color-changing zone changes color, it indicates that the ultraviolet intensity at this time is weak and does not have much effect on the human body; when only the first color-changing zone and the second color-changing zone change color, it indicates that the ultraviolet intensity at this time is medium and certain protective measures need to be taken; when all three color-changing zones change color, it indicates that the ultraviolet intensity at this time is strong and better protective measures should be taken, or avoid staying outdoors.

In the illustrated embodiment, the main body 11 comprises a housing 120 and a covering member 60, wherein the temperature regulation assembly that fulfills temperature regulation functions such as blowing air, contact cooling is mainly arranged on the housing 120. For example, the housing 120 is provided with an air inlet opening 15, an air outlet opening 16, and a heat dissipation opening 14, and an air channel connecting and communicating with the air inlet opening 15 and the heat dissipation opening 14. The main body 11 surrounds and defines the wear space 10. The air outlet opening 16 is arranged, for example, on a top wall of the main body 11. The color-changing body can be arranged on the top wall of the main body 11 or a side wall of the main body 11 away from the wear space 10.

The color-changing body can be arranged on the housing 120 and/or the covering member 60 in the above-discussed various ways of design. In the instant embodiment, the color-changing body is implemented as an ultraviolet color change layer 100 and is arranged on the top wall of the housing 120, and the color-comparing body is arranged at a position on an end portion of an outer surface of the light sensing layer 14 for easy observation and color comparison by the user. The ultraviolet color change layer 100 is provided with an air outlet hole 104 corresponding to the air outlet opening 16 of the housing 120 to avoid interference with air exiting.

In some embodiments, the color-changing body is arranged on the covering member 60, such as being arranged on the covering member 60 corresponding to the base portion 110, or being arranged on the covering member 60 corresponding to the arm portions 13. The color-changing body can be arranged in a text or graphic style to display product information, such as the brand name and product name of the product. Specifically, the arrangement can be made in such a way that the product information does not display if there is no sunlight or if the intensity of ultraviolet rays in the sunlight is low; and the product information changes to orange color to display if the intensity of ultraviolet rays in the sunlight is sufficient. Preferably, the color-changing body is arranged on the outer surface of the portion of the covering member 60 corresponding to the base portion 110 that protrudes to the bottom side by silk-screen printing, so as to facilitate external display toward the outside.

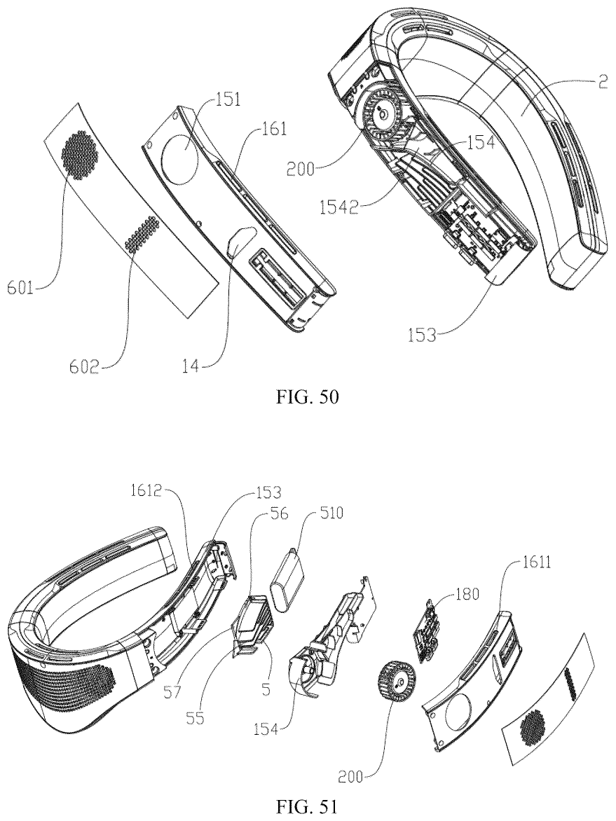

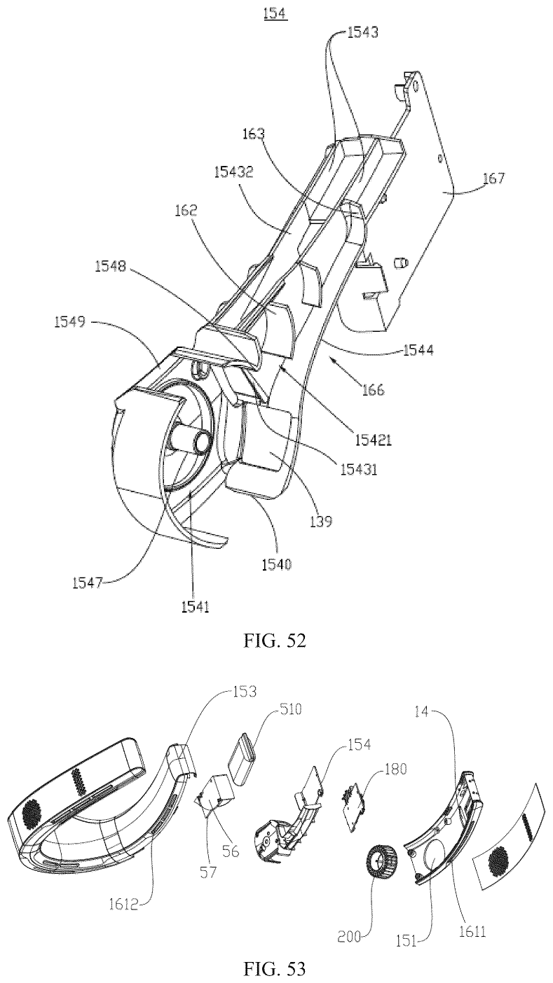

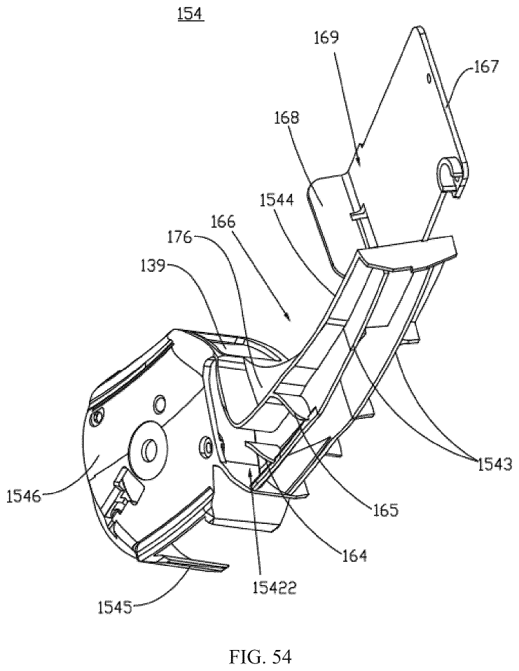

Referring to FIGS. 49-54, an eighth embodiment of the application provides a portable temperature regulation device, and compared with the sixth embodiment, further, in the instant embodiment, the portable temperature regulation device for example comprises: a first housing 153, a first internal seat 154, and a first fan 200.

In the instant embodiment, the portable temperature regulation device can be a handheld fan, a neck-mounted fan, a head-mounted fan, or a waist-mounted fan. For easy illustration, in the following embodiments, the neck-mounted fan is taken as an example for describing the portable temperature regulation device.

Specifically, the portable temperature regulation device further comprises: a connecting structure 400 and a second housing 155, wherein the number of the first housings 153 is two, and the two first housings 153 are respectively connected to two ends of the second housing 155 and the connection of each one is made with the connecting structure 400, that is, the two ends of the second housing 155 are each provided with one connecting structure 400, and connected by the connecting structure 400 to the first housing 153. The first housings 153 and the second housing 155 are suitable for wearing on the neck of the human body.

In the above, the first housing 153 is provided with a first air inlet opening 151 and a first air outlet opening 161. The first air inlet opening 151 is arranged, for example, on the side wall of the first housing 153 away from the human neck, and the first air outlet opening 161 is arranged, for example, on top wall of the first housing 153. The first internal seat 154 is mounted in the interior of the first housing 153. The first internal seat 154 is provided with a compartment 1541. The first air inlet opening 151 communicates with the compartment 1541. The first internal seat 154 is provided with an air guide channel 1542. The compartment 1541 communicates with the first air outlet opening 161 through the air guide channel 1542. The first fan 200 is disposed in the compartment 1541. Here, the air guide channel 1542 can be of a configuration of recess or a configuration of passageway. Specifically, in the instant embodiment, the air guide channel 1542 is of a configuration of recess. The first internal seat 154 is arranged in the interior of the first housing 153. Since the first internal seat 154 is provided with the compartment 1541 and the air guide channel 1542 communicating with each other, the first fan 200 is arranged in the compartment 1541 to form, in combination with the first internal seat 154, an air outlet module, so that airflow generated by the first fan 200 may quickly pass through the air guide channel 1542 to blow out from the first air outlet opening 161, and airflow may smoothly exit, and therefore, the problem of easily generating noise due to gaps resulting from a receiving housing and a driving fan being arranged separate from each other in the prior art can be solved. Further, the first fan 200 is mounted in the first internal seat 154 to form a standalone air outlet module, and this, compared with the prior art, requires no adjustment of the dimension matching relationship between the first fan 200 and the first housing 153 during the manufacturing, and thus, this kind of modularized design makes the product manufacturing easier.

In one preferred embodiment of the instant embodiment, the first internal seat 154 comprises a first portion and a second portion connected to each other. The first portion forms the compartment 1541. The second portion comprises a first air guide portion 1543 and a second air guide portion 1544. The first air guide portion 1543 is connected to one side of the second air guide portion 1544. The first air guide portion 1543 and the second air guide portion 1544 are both a plate-like structure and the two are arranged at an angle so as to form the configuration of recess of the air guide channel 1542.

In other embodiments, the first air guide portion 1543 and the second air guide portion 1544 may also both be a frame structure having a sideway opening, and the two are interlocked and connected to each other to thereby jointly form the configuration of passageway of the air guide channel 1542. Further, the first internal seat 154 needs to have an air passage opening formed in a position corresponding to the first air outlet opening 161. The air passage opening can be for example arranged at the connection between the first air guide portion 1543 and the second air guide portion 1544, so that airflow entering the air guide channel 1542 may be blown out of the first air outlet opening 161 through the air passage opening.

In one preferred embodiment of the instant embodiment, the first portion comprises a side wall 1545 and a bottom wall 1546 connected to each other. The side wall 1545 is arranged around a periphery of a bottom wall 1546 to cooperatively form the compartment 1541. The bottom wall 1546 is provided with a mounting pillar 1547 which is located in the compartment 1541. The first fan 200 can be for example a centrifugal fan, and the side wall 1545 is provided, corresponding thereto, with a scroll tongue portion 1548 which is arranged adjacent to the top wall of the first housing 153. An electric motor (not shown) of the first fan 200 is mounted on the mounting pillar 1547. Vanes of the first fan 200 are driven by the electric motor (not shown) to rotate and thus generate airflow. By fixing the electric motor of the first fan 200 to the mounting pillar 1547, the first fan 200 and the first internal seat 154 can form a module. When the first fan 200 is damaged after a long period of operation, the module formed by the first internal seat 154 and the first fan 200 can be quickly disassembled from the first housing 153 for replacement, which solves the problem in the prior art that the fan is not easy to disassemble and replace because the fan is fixed on the mounting pillar of the housing.

Further, the side wall 1545 is provided with a first avoiding notch 1549 and a second avoiding notch 1540, which are arranged opposite to each other. The connecting portion of the air guide channel 1542 and the compartment 1541 is located between the first avoiding notch 1549 and the second avoiding notch 1540. Specifically, the first avoiding notch 1549 is arranged adjacent to the top wall of the first housing 153, and the second avoiding notch 1540 is arranged adjacent to the bottom wall of the first housing 153. Compared to the situation where the first internal seat is not provided with the first avoiding notch 1549 and the second avoiding notch 1540 in the side wall 1545, with the diameter of the vanes of the first fan 200 being kept the same, arranging the first avoiding notch 1549 and the second avoiding notch 1540 in the side wall 1545 makes it possible to reduce the thickness between the top wall and the bottom wall of the first housing 153, and also, the top wall and the bottom wall of the first housing 153 do not cause interference with the vanes of the first fan 200, allowing the arrangement of the first fan 200 in the interior of the first housing 153 to be more compact.

In one preferred embodiment of the instant embodiment, the first air outlet opening 161 comprises a first sub air outlet opening 1611 and a second sub air outlet opening 1612, and the air guide channel 1542 comprises a first sub channel 15421 and a second sub channel 15422. The first sub channel 15421 and the second sub channel 15422 are respectively arranged at two opposite sides of the first air guide portion 1543. The first sub air outlet opening 1611 is set in communication with the compartment 1541 through the first sub channel 15421, and the second sub air outlet opening 1612 is set in communication with the compartment 1541 through the second sub channel 15422. By forming the first sub air outlet opening 1611 and the second sub air outlet opening 1612 that are arranged side by side in one side of the first housing 15, the discharging airflow range of the portable temperature regulation device can be expanded, and the user's experience is improved. Also, the first sub air outlet opening 1611 is connected to the compartment 1541 through the first sub channel 15421, and the second sub air outlet opening 1612 is connected to the compartment 1541 through the second sub channel 15422 which is separated from the first sub channel 15421, ensuring that the airflows passing through the first sub channel 15421 and the second sub channel 15422 do not interfere with each other.

Further, the first air guide portion 1543 comprises two sub air guide portions 15431 close to one end of the first fan 200 and two extension portions 15432 respectively connected to the two sub air guide portions 15431. Ends of the two sub air guide portions 15431 that are close to the first fan 200 are connected to each other to form a V-shaped air guide terminal, which is conducive to dividing the airflow generated by the first fan 200 into two parts that are respectively flowing to the first sub channel 15421 and the second sub channel 15422, thereby reducing the airflow resistance. The V-shaped air guide terminal is also connected to the side of the scroll tongue portion 1548 away from the first fan 200, so that the airflow can enter the first sub channel 15421 and the second sub channel 15422 more smoothly. The size of the distance between the two sub air guide portions 15431 in the width direction of the first housing 153 gradually increases in the direction from one end close to the first fan 200 toward another end away from the first fan 200, namely gradually increasing along the flowing direction of the airflow. The two extension portions 15432 extend along the length direction of the first housing 153 and are spaced from each other and parallel to each other.

Further, the first internal seat 154 further comprises a first air guide board 162, a first blocking portion 163, a second air guide board 164, and a second blocking portion 165. One side of the first blocking portion 163 is connected to one side of one of the extension portions 15432. The first blocking portion 163 extends obliquely along the thickness direction of the first housing 153. The end of the first blocking portion 163 away from the first fan 200 is arranged closer to the top wall of the first housing 153 relative to the other end of the first blocking portion 163 close to the first fan 200. The other end of the first blocking portion 163 close to the first fan 200 is connected to the end of the second air guide portion 1544 away from the first fan 200. After the airflow generated by the first fan 200 is blown into the first sub channel 15421, the airflow is blocked and guided by the first blocking portion 163 to blow out from the first sub air outlet opening 1611, so that the airflow cannot pass through the first blocking portion 163 to blow to the site further away from the first fan 200. There can be two first air guide boards 162, and the two first air guide boards 162 are arranged at intervals in the first sub channel 15421 and are located between the compartment 1541 and the first blocking portion 163. One side of the first air guide board 162 is connected to one side of one of sub air guide portions 15431. The first air guide board 162 extends obliquely along the thickness direction of the first housing 153. One end of the first air guide board 162 away from the first fan 200 is arranged closer to the top wall of the first housing 153 relative to the other end of the first air guide board 162 close to the first fan 200. An air passage gap is present between the other end of the first air guide board 162 close to the first fan 200 and the second air guide portion 1544. By arranging the first air guide boards 162 in the first sub channel 15421, airflow can be blown out uniformly from the first sub air outlet opening 1611.

One side of the second blocking portion 165 is connected to one side of another one of the sub air guide portions 15431. The second blocking portion 165 extends obliquely along the thickness direction of the first housing 153. One end of the second blocking portion 165 away from the first fan 200 is arranged closer to the top wall of the first housing 153 relative to the other end of the second blocking portion 165 close to the first fan 200. The other end of the second blocking portion 165 close to the first fan 200 is connected to the end of the second air guide portion 1544 away from the first fan 200. After the airflow generated by the first fan 200 is blown into the second sub channel 15422, the airflow is blocked and guided by the second blocking portion 165 to blow out from the second sub air outlet opening 1612, so that the airflow cannot pass through the second blocking portion 165 to blow to the site further away from the first fan 200. There can be for example one second air guide board 164, and the second air guide board 164 is arranged in the first sub channel 15421 and is located between the compartment 1541 and the second blocking portion 165. One side of the second air guide board 164 is connected to one side of another one of the sub air guide portions 15431. The second air guide board 164 extends obliquely along the thickness direction of the first housing 153. One end of the second air guide board 164 away from the first fan 200 is arranged closer to the top wall of the first housing 153 relative to the other end of the second air guide board 164 close to the first fan 20. An air passage gap is present between the other end of the second air guide board 164 close to the first fan 200 and the second air guide portion 1544. By arranging the second air guide board 164 in the second sub channel 15422, airflow can be blown out uniformly from the second sub air outlet opening 1612. In the above, as the volume of air entering the first sub channel 15421 is greater than the volume of air entering the second sub channel 15422, the length of the first sub channel 15421 can be set to be longer than the length of the second sub channel 15422, so that airflow can be blown out from the first sub air outlet opening 1611 to a wider range.

In one preferred embodiment of the instant embodiment, one side of the second air guide portion 1544 away from the first air guide portion 1543 forms, in combination with the first housing 153, a heat dissipation chamber 166. The first internal seat 154 is provided with an air passage opening 139 in communication with the heat dissipation chamber 166 and the compartment 1541. The air passage opening 139 can be arranged on the first portion, or the second portion, or the connection between the first portion and the second portion. The first housing 153 is also provided with a heat dissipation opening 14 in communication with the heat dissipation chamber 166. The heat dissipation opening 14 is arranged on the side wall of the first housing 153 away from the human neck. Specifically, heat generating components of the portable temperature regulation device, such as the battery 510, the circuit board 180, and the heat dissipating member 5, can be accommodated in the heat dissipation chamber 166. The airflow generated by the first fan 200 has a portion thereof passing through the air passage opening 139 to blow into the heat dissipation chamber 166, and the airflow blow the heat dissipating from the heat generating components out from the heat dissipation opening 14, so as to fulfill heat dissipation for the heat generating components, avoiding the problems that the temperatures of the heat generating components get excessively high to damage themselves or affect the temperature regulation performance of the portable temperature regulation device. In the above, the side wall of the first housing 153 away from the human neck may be further connected to a covering member 60. The covering member 60 is formed with a first through aperture array 601 at a location corresponding to the first air inlet opening 151, and the covering member 60 is formed with a second through aperture array 602 at a location corresponding to the heat dissipation opening 14. The first through aperture array 601 of the covering member 60 helps prevent hairs or clothing from being drawn into the first fan 200, and also, the second through aperture array 602 of the covering member 60 helps prevent the human body from accidentally contacting the heat generating components to get burned.

In one preferred embodiment of the instant embodiment, the portable temperature regulation device further comprises a heat dissipating member 5, a temperature regulation member 4, and a temperature conducting member 2. The heat dissipating member 5 is arranged in the heat dissipation chamber 166. The temperature conducting member 2 is fixedly connected to the first housing 153, and the temperature conducting member 2 is exposed outside the first housing 153. The temperature conducting member 2 is arranged, for example, on the side wall of the first housing 153 close to the human neck. The heat dissipating member 5 and the temperature conducting member 2 are separately set in thermal conduction connection with the temperature regulation member 4. The temperature regulation member 4 is specifically a semiconductor cooling sheet. The temperature conducting member 2 is an aluminum alloy temperature conducting member. When in operation, the temperature regulation member 4 transfer cold to the temperature conducting member 2 to be subsequently transferred by the temperature conducting member 2 to the human body for cooling the human body. At the same time, the heat generated by the temperature regulation member 4 is transferred to the heat dissipating member 5, and the heat dissipating member 5 dissipates the heat into the heat dissipation chamber 166. The first fan 200 generates an airflow and blows a portion of the airflow toward the heat dissipation chamber 166 to carry the heat dissipating from the heat dissipating member 5 out from the heat dissipation opening 14. Of course, the temperature regulation member 4 can also transfer the heat to the temperature conducting member 2, and the temperature conducting member 2 then transfers the heat to the human body for warming.

Further, the first internal seat 154 further comprises a mounting portion 167 and an isolating portion 168. The mounting portion 167 is connected to one end of the second air guide portion 1544 away from the first portion, and the mounting portion 167 and the first air guide portion 1543 are located on two opposite sides of the second air guide portion 1544. The isolating portion 168 is separately connected to the mounting portion 167 and the second air guide portion 1544. The isolating portion 168, the mounting portion 167, the second air guide portion 1544, and the first housing 153 together form a thermal isolation chamber 169 for accommodating a power supply. The isolating portion 168 is located between the thermal isolation chamber 169 and the heat dissipation chamber 166. The power supply is a battery 510, which is used to supply electrical power to power-consuming units, such as supplying electrical power to the first fan 200 and the temperature regulation member 4. Since the space inside the first housing 153 is limited, in order to prevent the heat of the heat dissipation chamber 166 from transferring to the power supply, the isolating portion 168 is used to separate the power supply and the heat dissipation chamber 166 to ensure that the power supply is not affected by the heat in the heat dissipation chamber 166.

In one preferred embodiment of the instant embodiment, the heat dissipating member 5 comprises a base plate 56, an extending part 57, and a heat dissipating plate 55. The heat dissipating plates 55 is connected to one side of the base plate 56 and is located in the heat dissipation chamber 166. The extending part 57 is connected to one side of the base plate 56 and is located between the temperature conducting member 2 and the second portion. The extending part 57 and the base plate 56 are both in thermal conduction connection with the temperature regulation member 4. Specifically, there are a plurality of heat dissipating plates 55, and the plurality of heat fins are arranged, at intervals, on one side of the base plate 56. By connecting the extending part 57 to one end of the base plate 56 and having the extending part 57 and the base plate 56 both connected to the temperature regulation member 4, the size of the temperature regulation member 4 can be made larger to increase its temperature regulating effect, and by the arrangement of the extending part 57 and the base plate 56, the heat conduction surface of the heat dissipating member 5 and the temperature regulation member 4 is increased to thereby improve the heat dissipation performance of the heat dissipating member 5 on the temperature regulation member 4.

Further, the first internal seat 154 is also provided with an avoiding opening 176. The avoiding opening 176 is located directly below the second sub channel 15422 adjacent to the first fan 200, and the avoiding opening 176 is in communication with the heat dissipation chamber 166. The extending part 57 is arranged in the avoiding opening 176 to prevent interference of the heat dissipating member 5 with the first internal seat 154, and the heat dissipating plates 55 can be entirely disposed in the heat dissipation chamber 166, so that the heat dissipating member 5 and the first internal seat 154 can be compactly installed in the interior of the first housing 153.

In one preferred embodiment of the instant embodiment, the temperature conducting member 2 is provided with a pearl finish layer, or alternatively, the temperature conducting member 2 is mixed with pearl powder. The pearl finish layer is mainly formed of pearl finish. Pearl finish is also referred to as mica paint and is a reflective coating, in which mica is used instead of aluminum particles, and mica pigment coated with titanium dioxide and iron oxide are added to the paint base. Light hitting on the mica particles induces refraction, so that the temperature conducting member 2 presents a cooler visual effect. The pearl powder can be pearl white (that is white pearl powder). Light hitting on the pearl power causes refraction, so as to also make the temperature conducting member 2 present a cooler visual effect.

In one preferred embodiment of the instant embodiment, a temperature-sensitive layer is arranged on the surface of the temperature conducting member 2. The temperature-sensitive layer can be a temperature-sensitive ink. The temperature-sensitive layer may present different colors according to the temperature change of the temperature conducting member 2, so as to facilitate the user to identify the temperature situation of the temperature conducting member 2 and to make the temperature conducting member 2 present a cooler visual effect. For example, when the temperature of the temperature conducting member 2 does not exceed 31° C., the temperature-sensitive layer appears blue, and when the temperature of the temperature conducting member 2 exceeds 31° C., the temperature-sensitive layer appears orange. Of course, the temperature-sensitive layer may also be set to present other color changes according to user needs.

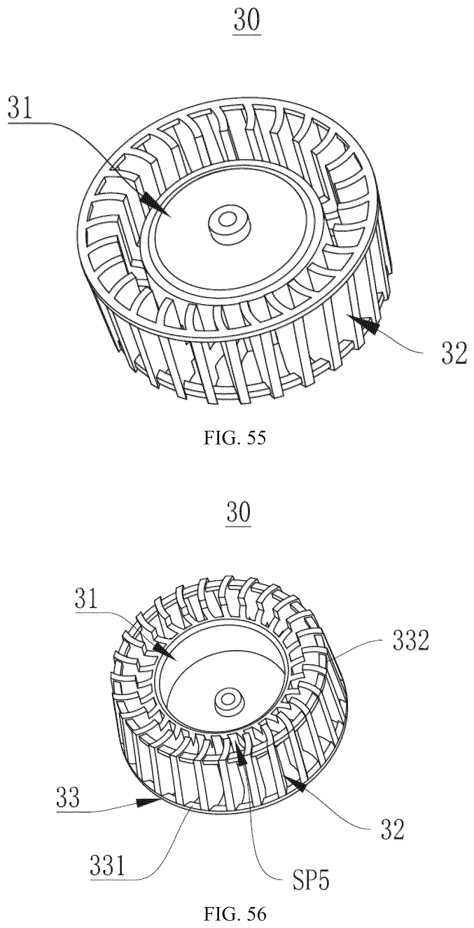

As shown in FIGS. 55 and 56, a ninth embodiment of the application provides a miniature centrifugal impeller 30, which can be used for fan, for example, in the portable temperature regulation device of any one of the first embodiment to the eighth embodiment discussed above. Specifically, the miniature centrifugal impeller 30 comprises for example: a hub 31 and a plurality of vanes 32. It is worth noting here that the miniature centrifugal impeller 30 refers to a small wind wheel that takes in air axially and discharges air radially, and uses centrifugal force to do work to increase the pressure of the air.

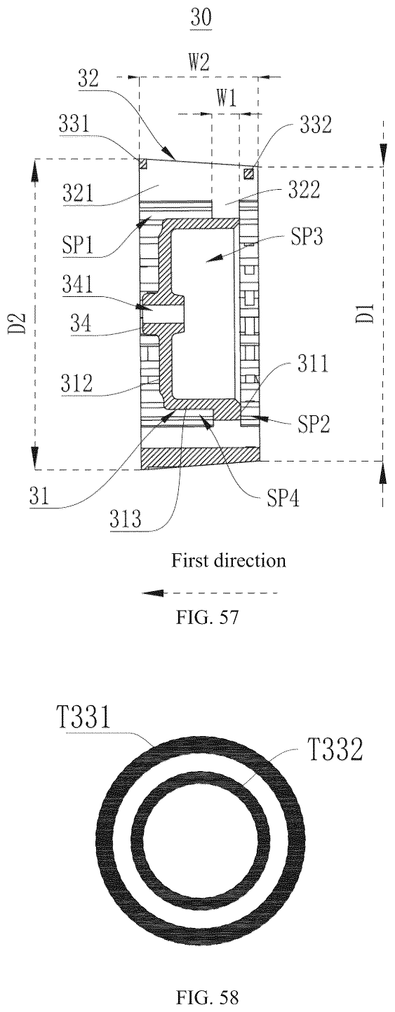

Specifically, the hub 31 is used, for example to provide support to the plurality of vanes 32, and the hub 31 is used, for example, to accommodate a rotor and a stator of a fan. As shown in FIG. 57, the hub 31 comprises for example a first end surface 311, a second end surface 312, and an outside surface 313. The first end surface 311 and the second end surface 312 are arranged opposite to each other in an axial direction of the impeller, and the outside surface 313 is connected between the first end surface 311 and the second end surface 312.

Continuing from the above, the plurality of vanes 32 are connected to the outside surface 313, and the plurality of vanes 32 are arranged around the hub 31. More specifically, the plurality of vanes 32 are sequentially spaced and arranged along the circumference of the outside surface 313. The plurality of vanes 32 extend beyond the second end surface 312 in a first direction from the first end surface 311 toward the second end surface 312 to enclose and form an air inlet space SP1, that is, the air inlet space SP1 is located on one side of the second end surface 312 away from the first end surface 311.

In this way, the embodiment of the application connects the plurality of vanes 32 to the outside surface 313 of the hub 31, and ensures the air inlet and outlet effects of the miniature centrifugal impeller 30 through the arrangement of the plurality of vanes 32 and the air inlet space SP1, and the arrangement of the hub 31 ensures that the miniature centrifugal impeller 30 can still install a miniature motor of a small-size centrifugal fan, and by eliminating the partition structure used in the prior art, the weight of the miniature centrifugal impeller 30 is reduced, thereby reducing the torque of the miniature motor, and then reducing the heat generation of the miniature motor or keeping the miniature motor at a stable speed, thereby ensuring the service life of the miniature centrifugal fan and the stability of the air outlet.

Further, as shown in FIG. 57, the contour size D2 of one end of the plurality of vanes 32 adjacent to the second end surface 312 is greater than the contour size D1 of one end of the plurality of vanes 32 adjacent to the first end surface 311. This arrangement allows the end of the plurality of vanes 32 adjacent to the second end surface 312 to have an enough space to set a larger air inlet space SP1. Further, along the first direction, the contour size of the plurality of vanes 32 is gradually increased, and in this way, a draft angle can be formed on the periphery of the plurality of vanes 32 to facilitate demolding of the miniature centrifugal impeller 30.

Further, as shown in FIG. 57, each of the vanes 32 comprises for example: a vane portion 321 and a connection portion 322. The vane portion 321 extends beyond the second end surface 312 along the first direction to enclose and form the air inlet space SP1. The connection portion 322 is connected between the vane portion 321 and the outside surface 313, and the connection portion 322 is connected to one end of the outside surface 313 adjacent to the first end surface 311. In the first direction, the size W1 of the connection portion 322 is smaller than the size W2 of the vane portion 321, so that an air inlet gap SP4 is formed between the vane portion 321 and the outside surface 313, and the air inlet gap SP4 is in communication with the air inlet space SP1. In this way, more airflow can enter the interior of the vane portion 321, increasing the air inlet airflow to facilitate the air blowing effect of the miniature centrifugal impeller 30. As shown in FIG. 56, an air passage gap SP5 is formed between the connection portions 322 of two adjacent vanes 3, and the air passage gap SP5 is in communication with the air inlet gap SP4. In this way, the plurality of connection portions 322 can also do work on the airflow to facilitate enhance the air blowing effect of the miniature centrifugal impeller 30.

In other embodiments of the application, as shown in FIG. 56, the miniature centrifugal impeller 30 further comprises a reinforcing rib 33. The reinforcing rib 33 are connected among the plurality of vane portions 321, so that the overall rigidity and strength of the plurality of vane portions 321 can be improved.

Continuing from the above, as shown in FIGS. 56 and 57, the reinforcing rib 33 comprises for example a first reinforcing rib 331, and the first reinforcing rib 331 is connected to one end of the plurality of vane portions 321 adjacent to the second end surface 312. Further, the reinforcing rib 33 may further comprise for example a second reinforcing rib 332, and the second reinforcing rib 332 is connected to one end of the plurality of vane portions 321 adjacent to the first end surface 311. A first projection area T331 that the first reinforcing rib 331 casts on a projection plane perpendicular to the first direction and a second projection area T332 that the second reinforcing rib 332 casts on the projection plane are staggered in position, that is the first projection area T331 and the second projection area T332 do not overlap. This way facilitates improvement of molding easiness of the first reinforcing rib 331 and the second reinforcing rib 332.

Further, as shown in FIG. 57, the plurality of vanes 32 extend beyond the first end surface 311 in a direction opposite to the first direction to enclose and form an avoiding space SP2, that is the avoiding space SP2 is located at one side of the first end surface 311 away from the second end surface 312. The avoiding space SP2 is used to accommodate a circuit board of the fan assembly, so as not only to effectively prevent the circuit board from contacting or scratching with the miniature centrifugal impeller 30 to avoid damage of product, but also to reduce the overall height of the miniature centrifugal fan.

Further, the hub 31 is formed with a disposition compartment SP3, and an opening of the disposition compartment SP3 is arranged in the first end surface 311. The disposition compartment SP3 is used to install the small-sized electric motor of the miniature centrifugal fan. Further, as shown in FIG. 57, a rotary axle mounting portion 34 is arranged in the middle position of the first end surface 311, and the rotary axle mounting portion 34 extends into the air inlet space SP1 along the first direction, so that the rotary axle mounting portion 34 has a sufficient height to install a rotary axle and to reduce the space of the disposition compartment SP3 occupied thereby to facilitate installation of the small-size electric motor of the miniature centrifugal fan in the disposition compartment SP3. The rotary axle mounting portion 34 is provided with a rotary axle through hole 341, and the rotary axle through hole 341 is used to mount the rotary axle to reduce radial shaking between the rotor and the stator.

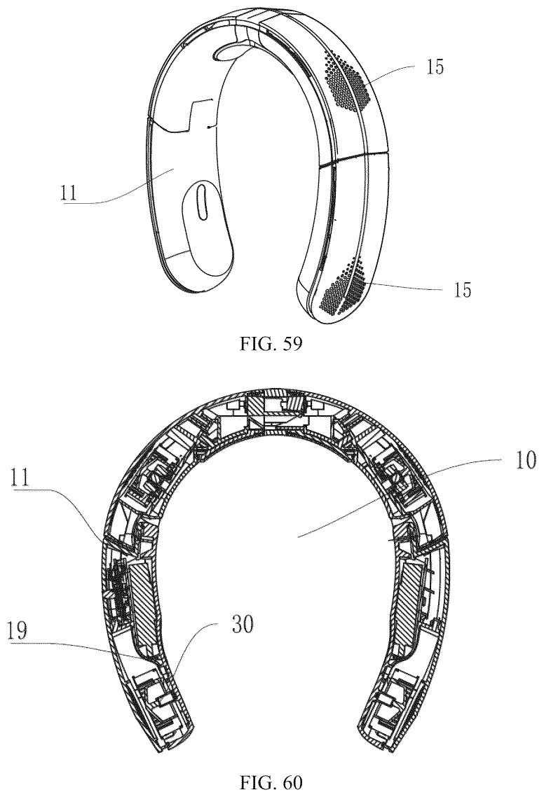

As shown in FIGS. 59 and 60, a tenth embodiment of the application provides a portable temperature regulation device. The portable temperature regulation device can be for example a neck-mounted fan, and can also be a handheld portable fan or other fans. Specifically, the portable temperature regulation device comprises for example: a main body 11 and one or more miniature centrifugal impellers 30. In this, the main body 11 is provided, in an interior thereof, with a volute 19. The miniature centrifugal impeller 30 is mounted in the volute 19. The miniature centrifugal impeller 30 can be for example the miniature centrifugal impeller provided in the above-described ninth embodiment.

Typically, the portable temperature regulation device further comprises a small-sized electric motor. The small-sized electric motor can be for example a stepping motor, for driving the miniature centrifugal impeller 30 to rotate. The small-sized electric motor may comprise for example a rotor and a stator. The stator is fixed on an inside wall of the main body 11, and the rotor is sleeved outside the stator and is rotatable relative to the stator. The rotor is connected to a hub 31 of the miniature centrifugal impeller 30.

Further, as shown in FIG. 60, the portable temperature regulation device can be for example a neck-mounted fan. Thus, the main body 11 is curved to form a wear space 10. An air inlet opening 15 is provided on one side of the main body 11 away from the wear space 10, and the air inlet opening 15 is in communication with the volute 19 and corresponds to an air inlet space SP1 of the miniature centrifugal impeller 30. When the miniature centrifugal impeller 30 rotates, the air inlet space SP1 forms into a low-resistance and high-negative-pressure space, so that airflow is drawn into the air inlet space SP1 from the air inlet opening 15. In the instant embodiment, the miniature centrifugal impeller 30 only takes in air from one single side, that is the airflow entering from the air inlet opening 15 only enters the air inlet space SP1 from the direction opposite to the first direction, so that the air inlet opening 15 is arranged on one single side on the main body 11 corresponding thereto. Specifically, arranging the air inlet opening 15 only on one single side of the main body 11 that is away from the wear space 10 may reduce the chance that the user's hairs are sucked into the air inlet opening 15 and the volute 19, reducing the possibility that the hairs are entangled in the miniature centrifugal impeller 30 to enhance the product safety. Further, as shown in FIG. 59, the number of the air inlet opening 15 can be plural, and the plural air inlet openings 15 are all arranged on one side of the main body 11 away from the wear space 10, that is the side of the main body 11 adjacent to the wear space 10 is provided with no air inlet opening 15.

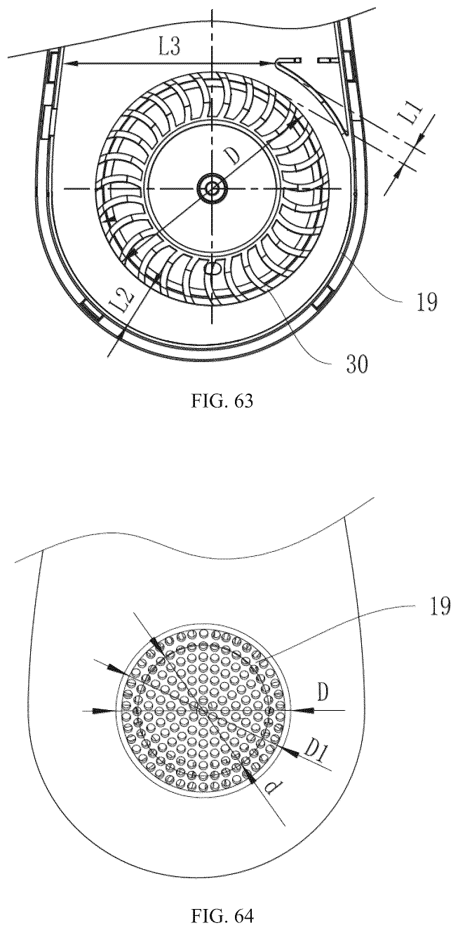

In an embodiment of the application, as shown in FIG. 62, a vane portion 321 of each of the vanes 32 has an outside edge 323 and an inside edge 324. In some embodiments, a curving direction of each vane portion 321 extending from the inside edge 324 to the outside edge 323 is along the rotation direction of the miniature centrifugal impeller 30; and correspondingly, the miniature centrifugal impeller 30 is a forward-inclined centrifugal impeller, and for the forward-inclined centrifugal impeller, the wind pressure of the vanes is large at a low speed, and a small-sized electric motor with a relatively small power can be used to drive the impeller, which is conducive to the miniaturization design of the centrifugal impeller. In addition, in combination with the assembled structure of the volute 19 and the miniature centrifugal impeller 30 shown in FIG. 63 it can be seen that starting from the scroll tongue, along the rotation direction of the miniature centrifugal impeller 30 (such as clockwise direction), the distance from the inside wall of the volute 19 to the outer circle with a diameter D of the miniature centrifugal impeller 30 is gradually increased, such as gradually increased from L1 to L2. When the miniature centrifugal impeller 30 is a forward-inclined centrifugal impeller, since the curving direction of each vane portion 321 is along the rotation direction of the miniature centrifugal impeller 30, airflow entering the miniature centrifugal impeller 30 is continuously enhanced in the rotation direction of the miniature centrifugal impeller 30 along the gradually increasing space between the miniature centrifugal impeller 30 and the inside wall of the volute 19 under the action of each vane portion 321, thereby further improving the outlet wind pressure of the miniature centrifugal impeller 30, thereby improving the outlet wind effect of the miniature centrifugal impeller 30.

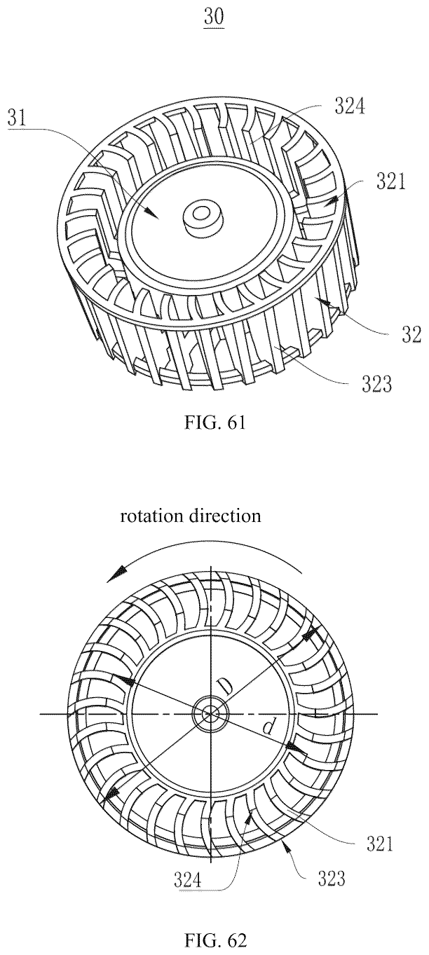

In some embodiments, referring to FIGS. 61, 62, and 63, the main body 11 is provided with the volute 19 formed in the interior thereof, and the miniature centrifugal impeller 30 is located in the volute 19. The ratio of distance L1 between the outer circle (having a diameter D) formed as being jointly enclosed by the outside edge 323 of each vane portion 321 and the scroll tongue of the volute 19 with respect to the diameter D of the outer circle is L1/D, wherein when L1 is small, the air volume is large but the noise is large (airflow impact being significant); oppositely, when L1 is large, the noise becomes small, but there will be circulation backflow and the air volume becomes smaller; therefore, the value range of L1/D is preferably 0.05-0.1, that is the value range of distance L1 is 0.05D-0.1D. On the other hand, the ratio of the outlet dimension L3 of the volute 19 with respect to the outer circle diameter D is L3/D, and here the outlet dimension L3 is the distance from the side wall of the outlet of the volute 19 that is opposite to the scroll tongue to the scroll tongue; wherein when L3 is small, the air outlet area is small, and conversely, when L3 is large, the scroll profile will become shorter and the pressure accumulation will be insufficient; therefore, the value range of L3/D is preferably 0.8-1.2, that is, the value range of the distance L3 is 0.8D-1.2D.

In some embodiments, referring to FIG. 64, the air inlet opening 15 is, for example, a mesh structure, and the outer edge diameter D1 of the projection area thereof on the projection plane perpendicular to the first direction is greater than the diameter d of the inner circle of the miniature centrifugal impeller 30 and less than the diameter D of the outer circle; in other words, the outer edge of the projection area that the air inlet opening 15 projected on the projection plane perpendicular to the first direction is located between the inside edge 324 and the outside edge 323 of each vane portion 321. In this way, the air inlet area of the miniature centrifugal impeller 30 can be effectively increased. Specifically, the conventional design size of the air inlet opening 15 of the miniature centrifugal impeller 30 is D1≤d, but the size of the miniature centrifugal impeller 30 is small, so that if the air inlet volume is to be guaranteed, the air inlet opening 15 needs to be opened larger; but the air inlet opening 15 cannot be opened too large to D1≥D, which will cause pressure relief, making the outlet wind pressure of the miniature centrifugal impeller 30 not strong.

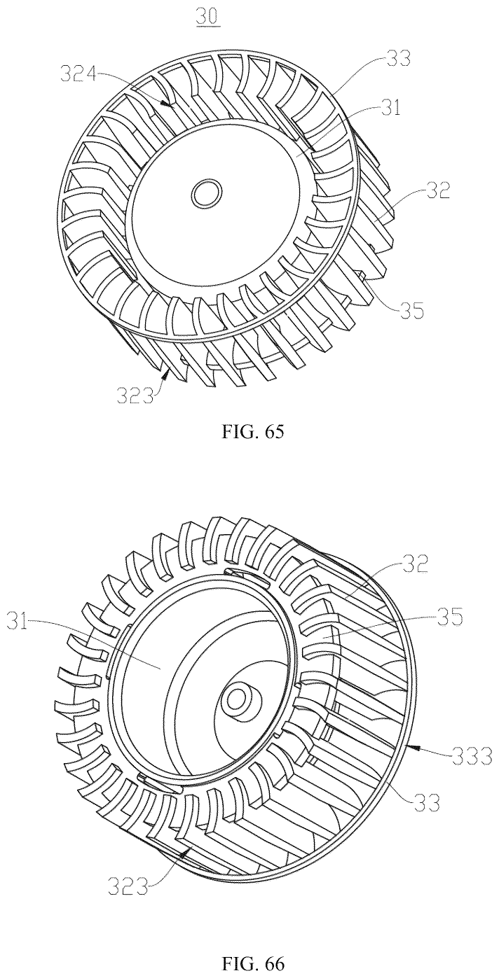

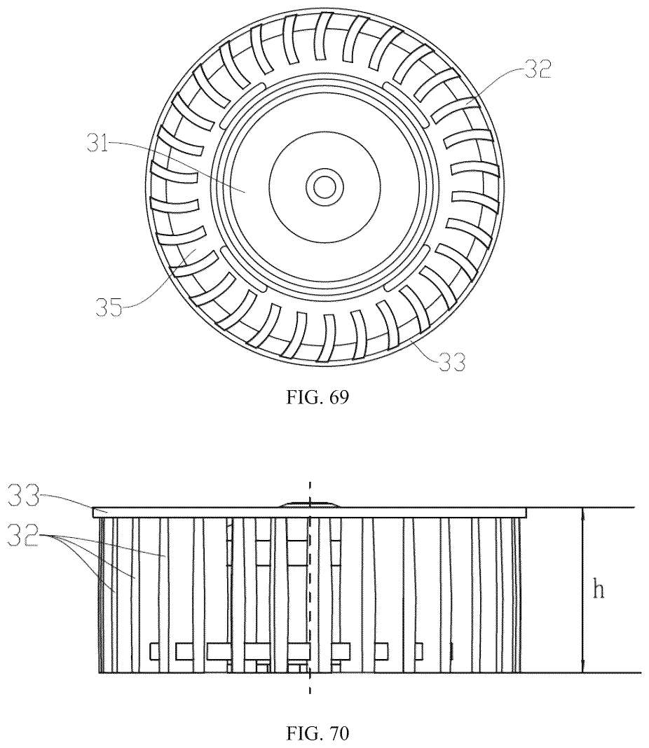

Referring to FIGS. 65-70, an eleventh embodiment of the application provides a miniature centrifugal impeller 30, which can be for example used in the portable temperature regulation device of any one of the first embodiment to the eighth embodiment discussed above. Specifically, the miniature centrifugal impeller 30 comprises: a hub 31 and a plurality of vanes 32 arranged around the periphery of the hub 31. For example, the miniature centrifugal impeller 30 is provided with a connecting disk 35 surrounding and connected to the hub 31, and the plurality of vanes 32 are arranged on the connecting disk 35 at intervals along a circumferential direction of the connecting disk 35 to thereby realize that the plurality of vanes 32 surround and are fixedly connected to the hub 31. In other embodiments, the plurality of vanes 32 can be directly connected and fixed to the circumferential side of the hub 31. It is worth noting here that the miniature centrifugal impeller 30 in the instant embodiment typically refers to a small-size impeller that takes in air axially and discharges air radially, and uses centrifugal force to do work to increase the pressure of air.

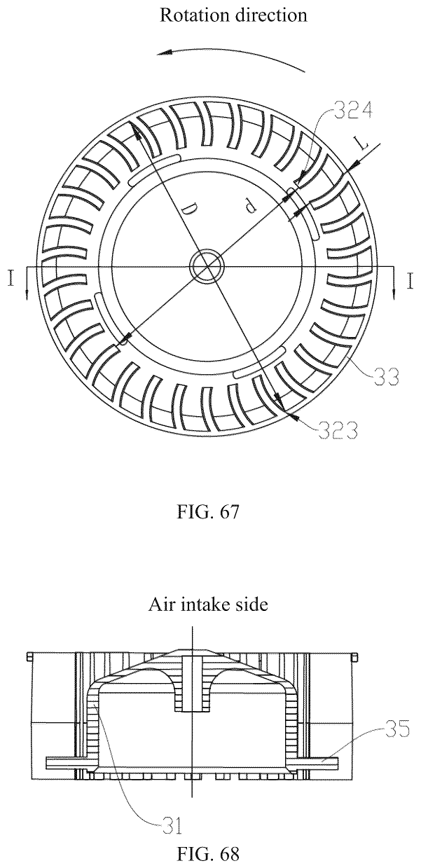

As shown in FIG. 67, the diameter of an inner circle jointly formed by the inside edges 324 of the plurality of vanes 32 is d, and the diameter of the outer circle jointly formed by the outside edges 323 of the plurality of vanes 32 is D. Here, the ratio d/D of the inner circle diameter d with respect to the outer circle diameter D is also referred to as a wheel diameter ratio (that is the inner and outer circle diameter ratio). The Applicant of the application has repeatedly conducted design optimization and proof-testing for miniature centrifugal impellers of which the outer circle diameter D is smaller than 50 mm and found that b controlling the wheel diameter ratio d/D of the miniature centrifugal impeller 30 to have a value range of 0.5-0.85 and controlling the chord length L of each vane 32 to have a value range of 3.75 mm-10.75 mm, it is possible to ensure the effective working area size of each vane 32, and the outlet wind pressure of the operation of the miniature centrifugal impeller can be enhanced to thereby effectively solve the problem of poor air outlet effect of the miniature centrifugal fans. Here, the chord length Lis defined as a straight lien distance between the inside edge 324 and the outside edge 323 of the vane 32 (that is the straight line distance between the inner and outer edge). In some embodiments, each vane 32 has the same chord length L. Further, the outer circle diameter D of the miniature centrifugal impeller 30 has a value range of for example 28 mm-47 mm. More specifically, although reducing the wheel diameter ratio d/D allows each vane 32 to have an increased effective working area size, yet if the wheel diameter ratio d/D is designed to be too small, for example, less than 0.5, it will cause the inner side space of the inside edge 324 of each vane 32 to be too small. On the one hand, the air inlet space of the miniature centrifugal impeller is too small, resulting in insufficient air intake, thereby affecting the air outlet effect, and on the other hand, it may even make the placement space of the hub insufficient, resulting in the structural design of the hub becoming complicated, and therefore, the embodiment of the application sets the lower limit of the wheel diameter ratio to 0.5 and the upper limit of the chord length to 10.75 mm; oppositely, although increasing the wheel diameter ratio d/D increases the inside space of the inside edge 324 of each vane 32 to thereby increase the air inlet space of the miniature centrifugal impeller to obtain a larger air inlet volume, yet is the wheel diameter ratio d/D is designed to be too large, such as larger than 0.85, it will make the chord length of each vane too short, such as less than 3.75 mm, resulting in the effective working area size of each vane too small, thereby causing insufficient air outlet wind pressure of the miniature centrifugal impeller, therefore affecting air outlet effect, and therefore, the embodiment of the application sets the upper limit of the wheel diameter ratio to 0.85 and the lower limit of the chord length to 3.75 mm.

In some embodiments, the wheel diameter ratio d/D of the miniature centrifugal impeller 30 is in the range of 0.6-0.8, so as to further ensure the effective working area size of each vane 32, thereby increasing the outlet air wind pressure of the miniature centrifugal impeller 30 to enhance the outlet air effect of the miniature centrifugal impeller 30. In a preferred embodiment, the wheel diameter ratio d/D of the miniature centrifugal impeller 30 is 0.7.

In some embodiments, as shown in FIGS. 70, each vane 32 has the same height h in the axial direction of the hub 31, and the ratio h/D of the height h to the outer circle diameter D is of a value range of for example 0.2-0.5, that is, the range of the height h is 0.2D-0.5D; his range is conducive to the thinned design of the miniature centrifugal impeller 30 to be easily used in portable electronic devices. Further, the ratio h/D of the height h to the outer circle diameter D is in the range of 0.25-0.4, that is the range of the height h is 0.25D-0.4D, preferably 0.3D.

In some embodiments, as shown in FIGS. 65, 66, and 68, the miniature centrifugal impeller 30 may further comprises a reinforcing rib 33, and the reinforcing rib 33 is arranged at one end of the plurality of vanes 32 adjacent to the air inlet side of the miniature centrifugal impeller 30 (or referred to as the vane front end); the outermost edge 333 of the reinforcing rib 33 is flush with the outside edge 323 of the plurality of vanes 32 or protrudes beyond the outside edge 323 of the plurality of vanes 32 is the radial direction of the hub 31, so that the reinforcing rib 33 may connect the vane front end of each vane 32 together to avoid deformation of the vanes 32 and also to make the reinforcing rib 33 not to cover the vane front end of each vane 32 as much as possible, and thus, the top of each vane 32 can be used as an air inlet area to increase the air inlet area of the entire miniature centrifugal impeller 30. In a preferred embodiment, as shown in FIG. 66, the inside surface of the reinforcing rib 33 is fixedly connected to the outside edges 323 of the plurality of vanes 32, and the outermost edge 333 of the reinforcing rib 33 is located outside the outside edges 323 in the radial direction of the hub 31, that is the reinforcing rib 33 extends outwards by a non-zero distance relative to the outside edges 323 in the radial direction of the hub 31, so that the vane front end face of each vane 32 is completely covered by the reinforcing rib 33. Further, in one embodiment, it can be seen by jointly referring to FIG. 70 that the top surface of the reinforcing rib 33 (that is the surface adjacent to the air inlet side of the centrifugal fan 10) is flush with the vane front end face of each vane 32.

In some embodiments, as shown in FIGS. 65 and 67, the number of the plurality of vanes 32 arranged on the same side of the connecting disk 35 is greater than or equal to 25 and less than or equal to 35, preferably 30 or 32. With such a design, the wind resistance and the outlet wind pressure of the miniature centrifugal impeller can be reasonably controlled. In this, if the number of the vanes 32 is large, the miniature centrifugal impeller is heavy and the wind resistance is great; if the number of the vanes 32 small, although the wind resistance is small, yet the overall effective working area of the miniature centrifugal impeller is small, and the outlet wind pressure is insufficient.

In some embodiments, as shown in FIGS. 65-67, the curving direction of each vane 32 from the inside edge 324 to the outside edge 323 is along the rotation direction of the miniature centrifugal impeller 30; correspondingly, the miniature centrifugal impeller 30 is a forward-inclined centrifugal impeller, and for the forward-inclined centrifugal impeller, the outlet wind pressure of the vanes is large at a low speed, and an electric motor with a relatively small power can be selected, which is conducive to the miniaturization design of the centrifugal impeller.

In some embodiments, as shown in FIG. 68, a conical structure is provided on one side of the hub 31 adjacent to the air inlet side of the miniature centrifugal impeller 30 (or referred to as the hub front side). Here, the conical structure is used as a flow guide structure to prevent the airflow from forming a vortex at the air inlet opening of the miniature centrifugal impeller 30, so that the airflow can enter the interior of the miniature centrifugal impeller 30 more uniformly and noise at the air inlet opening can be reduced.

Further, in one embodiment, the miniature centrifugal impeller 30 is an integrally formed structure, and the material used may be plastics; however, the embodiment of the present application is not limited thereto.

In summary, the miniature centrifugal impeller of the instant embodiment reasonably controls the value range of the wheel diameter ratio d/D and controls the value range of the chord length L of each vane, and the parameter of the miniature centrifugal impeller is defined as the wheel diameter ratio being greater than or equal to 0.5 and less than or equal to 0.85, and the vane chord length being greater than or equal to 3.75 millimeters and less than or equal to 10.75 millimeters, so as to ensure the effective working area size for each of the vanes to thereby ensure the outlet wind pressure of the miniature centrifugal impeller, and thus making the miniature centrifugal fan have a high-quality air outlet effect.

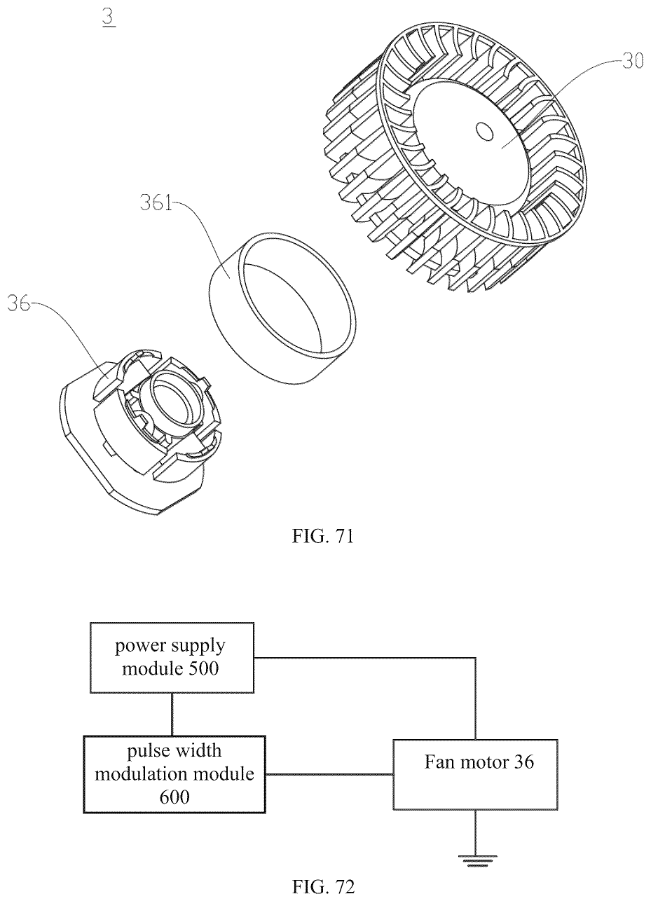

Referring to FIG. 71, a twelfth embodiment of the application provides a centrifugal fan 3, which comprises an electric motor 36 and a miniature centrifugal impeller 30. The electric motor 36 is mechanically coupled to the miniature centrifugal impeller 30. In the instant embodiment, the specific structure of the miniature centrifugal impeller 30 of the centrifugal fan 3 may refer to the corresponding structure of the above-discussed eleventh embodiment shown in FIGS. 65-70, and repeated description will be omitted herein.

Continuing from the above, the electric motor 36 can adopt a single-phase two-wire motor or a three-phase three-wire motor, and preferably adopts a three-phase three-wire motor. Compared with a single-phase two-wire motor, the three-phase three-wire motor has low power, low noise and energy saving. Further, a magnetic ring 361 (that is a rotor) of the electric motor 36 is suitable for being accommodated in a hub 31 of the miniature centrifugal impeller 30 (see FIG. 66), which can be a rubber magnetic ring or a neodymium iron boron magnetic ring, and preferably a neodymium iron boron magnetic ring. The magnetic ring adopts a neodymium iron boron magnetic ring, and compared with a rubber magnetic ring, thickness can be made thinner for the same magnetic flux, providing a larger space for the electric motor, so that the electric motor can be set as a three-phase three-wire motor to reduce power. For example, the thickness of a rubber magnetic ring is often greater than 1 mm, while the thickness of a neodymium iron boron magnetic ring can be less than 0.8 mm. More specifically, the single-phase two-wire motor generally refers to a small-power single-phase asynchronous motor powered by a single-phase AC power supply. This type of motor usually has a two-phase winding on the stator. The distribution of the two-phase winding on the stator and the power supply condition are different and may produce different starting characteristics and operating characteristics. The three-phase three-wire motor has a symmetric three-phase winding on a stator, and a rotor is made up of fixed-polarity magnetic poles with the same number of poles as the stator winding. The fixed-polarity magnetic poles are generated by the DC current fed into the magnetic pole excitation winding. When the symmetric three-phase winding of the motor stator is connected to a symmetrical three-phase power supply and a symmetrical three-phase current flows therethrough, a rotating magnetic field with the same number of poles as the rotor will be generated in the air gap of the electric motor. The magnetic poles of the rotating magnetic field will attract the rotor magnetic poles to rotate at the same synchronous speed according to the principle of opposites attracting.

Further, the embodiment of the application also provides a portable electronic device, which may comprise for example a housing and a centrifugal fan accommodated in the housing. Here, the centrifugal fan may adopt the centrifugal fan 3 described with reference to FIG. 71, so that the specific structure will not be repeatedly described herein. For the portable electronic device, the size of a receptacle space in the interior of the housing is generally limited, and to realize the functions of air blowing and heat dissipating thereof, it is inevitable to use a centrifugal fan with a miniature centrifugal impeller.

As shown in FIG. 72, a thirteenth embodiment of the application provides a fan speed regulation circuit, which can be used in the portable temperature regulation device of any one of the first embodiment to the eighth embodiment described above to realize a fan speed regulation function. The fan speed regulation circuit comprises a power supply module 500, a pulse width modulation module 600, and a fan motor 36. The power supply module 500 comprises a first output terminal, and the power supply module 500 is used to output a predetermined value of voltage through the first output terminal. The pulse width modulation module 600 is electrically connected to the power supply module 500. The pulse width modulation module 600 comprises a second output terminal. The pulse width modulation module 600 is used to output a pulse width modulation signal through the second output terminal. The fan motor 36 comprises a motor negative electrode, a motor positive electrode, and a motor control terminal. The motor negative electrode is grounded. The motor positive electrode is electrically connected to the first output terminal. The motor control terminal is electrically connected to the second output terminal. The pulse width modulation signal is also referred to as PWM signal. The power supply module 500 is connected to the motor positive electrode to provide a constant value of voltage to the fan motor 36, and there is no need to set up a boost circuit separately, so as to simplify the circuit design and save circuit preparation cost. By outputting PWM signals with different duty cycles from the pulse width modulation module 600, different rotation speeds of the fan motor 36 can be achieved, and since the voltage that the power supply module 500 outputs to the motor positive electrode is kept constant (such as being kept constant at 5V), the adjustment of the rotation speed can be more precise and stepless adjustment of the rotation speed can be realized, improving the endurance performance. In the instant embodiment, the number of the fan motor 36 can be one, or two, or any other number, which is not limited in the embodiment. Multiple fan motors 36 can be connected in parallel.

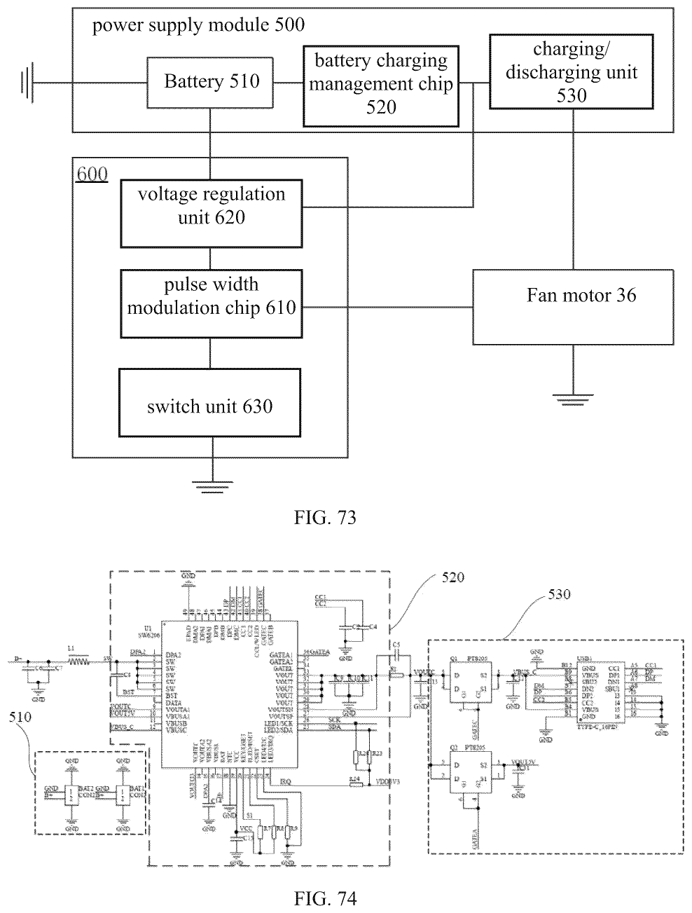

In the above, referring to FIG. 73, the power supply module 500 may specifically comprises for example a battery 510, a battery charging management chip 520, and a charging/discharging unit 530. The battery 510 comprises a battery positive electrode and a battery negative electrode. The battery negative electrode is ground. The charging/discharging unit 530 is connected to the battery positive electrode and comprises the first output terminal mentioned above. The charging/discharging unit 530 is used to carry out charging or discharging for the battery 510. The battery charging management chip 520 is electrically connected between the battery positive electrode and the charging/discharging unit 530 and is used to subject a current that is inputted during charging of the battery 510 to step-down processing for outputting to the battery positive electrode, or is used to subject a current that is inputted during discharging of the battery 510 to step-up processing for outputting to the charging/discharging unit 530. More specifically, for example referring to FIG. 74, the charging/discharging unit 530 specifically comprises a charging socket USB1, a first field effect transistor Q1, and a second field effect transistor Q2. The charging socket USB1 is used to connect with an external power supply. The source of the first field effect transistor Q1 is electrically connected to the charging socket USB1, and the drain of the first field effect transistor Q1 is electrically connected to the battery charging management chip 520. The drain of the second field effect transistor Q2 is electrically connected to the drain of the first field effect transistor Q1, and the source of the second field effect transistor Q2 is electrically connected to the first output terminal. The battery charging management chip 520 can be for example a SW6206 type management chip. When the battery 510 is charged, a current is inputted from the external power supply through the charging socket USB1, and inputted through the first field effect transistor Q1 to the battery charging management chip 520 to be input, after being stepped down by the battery charging management chip 520, to the battery positive electrode B+. When the battery 510 is discharged, a current is outputted from the battery positive electrode B+, and, after being stepped up by the battery charging management chip 520, and a predetermined value of voltage is outputted through the source of the second field effect transistor Q2 to the first output terminal to provide a constant voltage to the fan motor 36. Of course, as shown in FIG. 74, an electrical resistor R1 may be connected between the charging/discharging unit 530 and the battery charging management chip 520, and an electrical inductor L1 may be connected between the battery charging management chip 520 and the battery 510. The instant embodiment does not describe the specific components in the battery charging management chip 520 and the charging/discharging unit 530 one by one, and the design of the power supply module 500 may refer to the specific circuit diagram shown in FIG. 74, or can be set with reference to other power supplies that can provide a constant voltage. The instant embodiment is not limited to this.

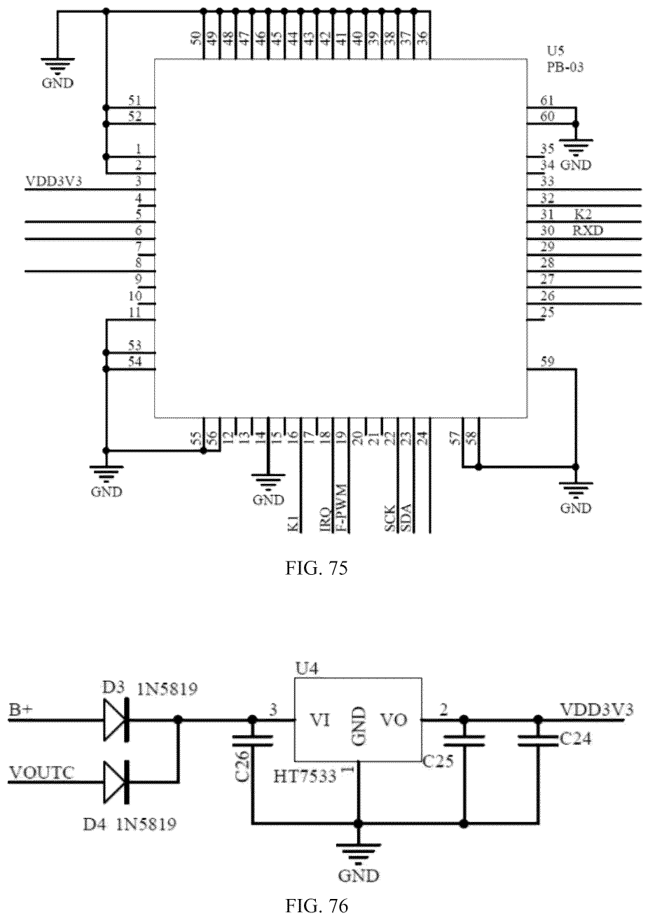

In one embodiment, further referring to FIG. 73, the pulse width modulation module 600 may comprises for example a pulse width modulation chip 610. The pulse width modulation chip 610 comprises a second output terminal used to output the pulse width modulation signal. Referring for example to FIG. 75, the pulse width modulation chip 610 can be for example a PB-03 chip, in which the 19^{th} pin is the second output terminal, which outputs the pulse width modulation signal to the motor control terminal of the fan motor 36.

Further, the pulse width modulation chip 610 further comprises a first input terminal, and the pulse width modulation module 600 further comprises a voltage regulation unit 620, which is electrically connected between the power supply module 500 and the first input terminal and is used to supply a stable input voltage to the pulse width modulation chip 610. As specifically shown in FIGS. 75, the third pin of the pulse width modulation chip 610 is the first input terminal.

For example, the circuit structure of the voltage regulation unit 620 may refer for example to FIG. 76, specifically comprising a first Schottky diode D3, a second Schottky diode D4, a voltage regulation chip U4, a first electrical capacitor C26, a second electrical capacitor C25, and a third electrical capacitor C24. The positive terminal of the first Schottky diode D3 is electrically connected to the battery positive electrode. The positive terminal of the second Schottky diode D4 is electrically connected between the charging/discharging unit 530 and the battery charging management chip 520. The negative terminal of the second Schottky diode D4 is electrically connected to the negative terminal of the first Schottky diode D3. The voltage regulation chip U4 comprises an input pin, an output pin, and a grounding pin. The input pin of the voltage regulation chip U4 is connected to the negative terminal of the first Schottky diode D3. The output pin of the voltage regulation chip U4 is connected to the first input terminal of the pulse width modulation chip 610 (which is the third pin of the pulse width modulation chip 610 shown in FIG. 75. The grounding pin of the voltage regulation chip U4 is grounded. One end of the first electrical capacitor C26 is electrically connected to the input pin of the voltage regulation chip U4 and another end grounded. One end of the second electrical capacitor C25 is electrically connected to the output pin of the voltage regulation chip U4 and another end grounded. The third electrical capacitor C24 is connected in parallel with the second electrical capacitor. The first Schottky diode D3 and the second Schottky diode D4 can be for example an IN5819 type diode. The voltage regulation chip U4 can be for example a HT7533 type chip. The positive terminal of the second Schottky diode D4 can be for example specifically connected to the VOUTC pin between the electrical resistor R1 and the charging/discharging unit 530 shown in FIG. 74.

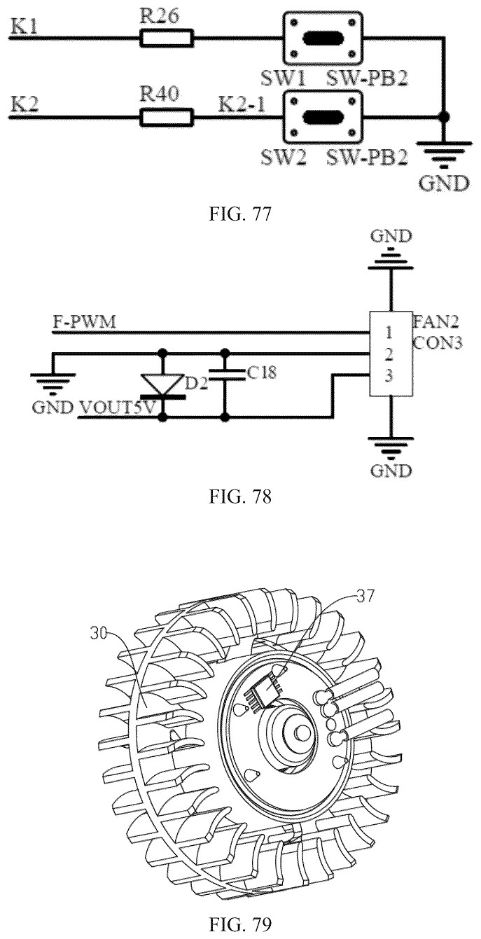

More specifically, the pulse width modulation module 600 further comprises for example a switch unit 630, which is used to adjust the pulse width modulation signal outputted from the pulse width modulation chip 610. The switch unit 630 comprises a first connection terminal and a second connection terminal. The first connection terminal is electrically connected to the pulse width modulation chip 610, and the second connection terminal is grounded. Specifically referring to FIG. 77, the first connection terminal of the switch unit 630 comprises a first switch pin and a second switch pin, and correspondingly, the pulse width modulation chip 610 comprises a first control pin and a second control pin. The first switch pin is connected to the first control pin, and the second switch pin is connected to the second control pin. The switch unit 630 further comprises a first switch SW1 and a second switch SW2. The first switch is electrically connected between the first switch pin and the second connection terminal, and the second switch is electrically connected between the second switch pin and the second connection terminal. The pulse width modulation chip 610 is used to control starting and stopping of the fan motor 36 in response to an action of the first switch. The pulse width modulation chip 610 is also used to control the duty ratio of the pulse width modulation signal in response to an action of the second switch. Comparing FIG. 75 and FIG. 77, that is the 16th pulse width modulation chip 610 is connected to the first switch pin, and the first switch SW1 and the second switch SW2 are for example specifically a SW-PB2 type pushbutton switch. One end of the first switch SW1 is connected to the 16th pin of the pulse width modulation chip 610 and another end grounded. The 31st pin of the pulse width modulation chip 610 is connected to the second switch pin. One end of the second switch SW2 is connected to the 31st pin of the pulse width modulation chip 610 and another pin grounded. As shown in FIG. 77, the switch unit 630 further comprises for example an electrical resistor R26 arranged between the first switch pin and the first switch SW1 and an electrical resistor R40 arranged between the second switch pin and the second switch SW2. In actual use, if it needs to start or stop the fan motor 36, the first switch SW1 is operated, and if it needs to adjust the rotation speed of the fan motor 36, the second switch SW2 is operated. The operation is easy.

In one embodiment, referring to FIG. 78, the fan speed regulation circuit further comprises a motor protection unit connected in parallel between the motor positive electrode and the motor negative electrode of the fan motor 36. The motor protection unit comprises a diode D2 and a protection capacitor C18. The positive terminal of the diode D2 is electrically connected to the motor negative electrode, and the negative terminal the diode D2 is electrically connected to the motor positive electrode. The protection capacitor C18 and the diode D2 are connected in parallel between the motor positive electrode and the motor negative electrode. The motor protection unit can consume the electric energy generated when the fan motor 36 stops working, perform energy recovery, and prevent damage to the fan motor 36.

Another embodiment of the application provides a portable speed regulation fan, which comprises the fan speed regulation circuit described in any of the previous embodiments. The portable speed regulation fan further comprises for example an external housing in which the fan speed regulation circuit is accommodated and vanes fixed on the fan motor 36, and can refer to the assembly method of a conventional portable fan, which is not limited in this embodiment. By adopting the previously described fan speed regulation circuit, the step for the wind speed adjustment of the speed adjustable fan can reach 1%, achieving an effect of stepless speed adjustment, making the speed adjustment more flexible and more convenient to use. In addition, there is no need to add a separate boost circuit, and the cost can also be greatly reduced.

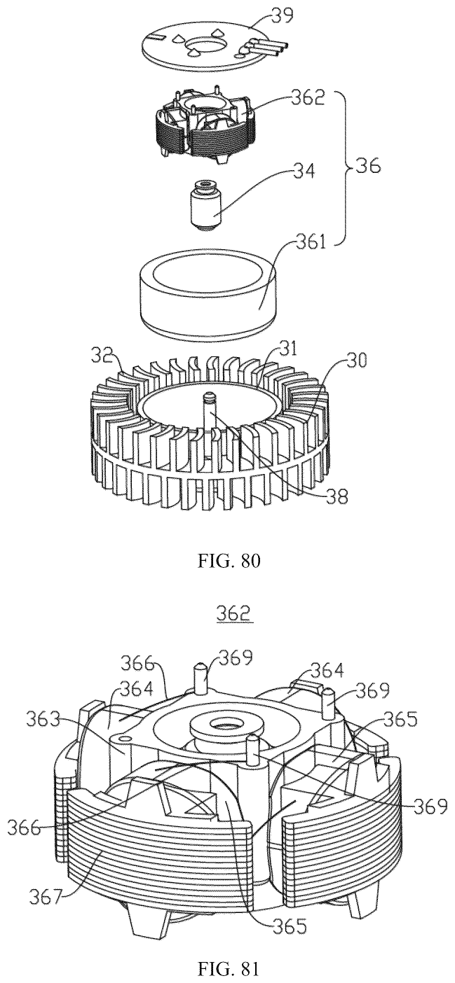

Referring to FIGS. 79 and 80, a fourteenth embodiment the application provides a fan assembly, which can be for example the centrifugal fan used in any of the first embodiment to the eighth embodiment described above. Specifically, the fan assembly comprises: an impeller 30, an electric motor 36, and a control chip 37. The electric motor 36 can comprise for example a stator 362 and a rotor 361. The rotor 361 is rotatable relative to the stator 362 The stator 362 comprises at least two pairs of windings. The rotor 361 is arranged around the periphery of the at least two pairs of windings, and the rotor 361 is connected to the impeller 30 such that the impeller 30 is rotatable together with the rotor 361. The control chip 37 is electrically connected to the at least two pairs of windings, and the control chip 37 is used to control the rotor 361 to drive the impeller 30 to rotate by controlling the magnetic field changes on the at least two pairs of windings. The impeller 30 may comprise for example a hub 31 and vanes 32 arranged on the hub 31. The impeller 30 may also be provided with, for example, a rotary axle 38. The electric motor 36 may also comprise for example a rotary axle mounting portion 34. A first end of the rotary axle 38 is connected to the impeller 30, and a second end of the rotary axle 38 is inserted into the rotary axle mounting portion 34. The second end of the rotary axle 38 is rotatable in the rotary axle mounting portion 34 relative to the rotary axle mounting portion 34. The fan assembly may also comprise for example a fixed axle. The fixed axle is provided with for example an axle hole, and the rotary axle mounting portion 34 is mounted in the axle hole. The second end of the rotary axle 38 is rotatable relative to the fixed axle in the rotary axle mounting portion 34.

The rotor 361 can be for example accommodated in the hub 31 of the impeller 30. The rotor 361 can be, for example, a rubber magnetic ring or a neodymium iron boron magnetic ring. Preferably, the rotor 361 is a neodymium iron boron magnetic ring. Compared with the rubber magnetic ring, the rotor 361 using the neodymium iron boron magnetic ring has a thinner thickness for the same magnetic flux, and can provide a larger space for the stator 362, so that the stator 362 can be set as a three-phase three-wire motor to reduce power. The thickness of the rubber magnetic ring is usually greater than 1 mm, and the thickness of the neodymium iron boron magnetic ring can be less than 0.8 mm. Preferably, the thickness of the rotor 361 provided in the instant embodiment is less than 0.8 mm.

Referring to FIGS. 81 and 82, the stator 362 may further comprise for example a motor bracket 363, and the at least two pairs of windings are arranged on the motor bracket 363. The at least two pairs of windings may comprise for example two pairs of windings, and each pair of windings comprises a first winding 364 and a second winding 365. For example, the two pairs of windings may comprise for example a pair of primary windings and a pair of secondary windings, and one pair of primary windings comprises a first winding 364 and a second winding 365, and one pair of secondary windings also comprises a first winding 364 and a second winding 365. As shown in FIG. 81, one pair of primary windings and one pair of secondary windings are arranged perpendicularly to each other on the motor bracket 363, that is the two first windings 364 of the primary windings and the secondary windings are arranged adjacent to each other, and the two second windings 365 of the primary windings and the secondary windings are arranged adjacent to each other. The stator 362 may comprise for example a first winding 366 and silicon steel sheets 367. The motor bracket 363 may be provided with for example four wire winding portions 368. The first winding 366 may, for example, be an enameled wire. Enameled wire is a main type of winding wire, consisting of a conductor and an insulating layer and is formed by softening a bare wire through annealing, followed by multiple times of painting and baking. The silicon steel sheets 367, also known as electrical steel sheets or silicon steel sheets, refers to a silicon-iron soft magnetic alloy with an extremely low carbon content (the carbon content being less than 0.005% after annealing) and a silicon content generally 0.5%-4.5%. Adding silicon to iron can increase its resistivity and maximum magnetic permeability. The silicon steel sheets 367 may, for example, have multiple layers and be connected to the motor bracket 363. A portion of the silicon steel sheets 367 may, for example, be wrapped in the four wire winding portions 368 of the motor bracket 363, and outer side of the silicon steel sheets 367 protrudes outside the motor bracket 363, and the inner side of the silicon steel sheets 367 protrudes into a middle hole of the motor bracket 363. The rotary axle mounting portion 34 is accommodated in the middle hole of the motor bracket 363 and is separated from the inner side of and the silicon steel sheets 367. The fixed axle is clamped and fixed between the rotary axle mounting portion 34 and the inner side of the silicon steel sheets 367. The first winding 366 is sequentially wrapped around the four wire winding portions 368 to form a pair of primary windings and a pair of secondary windings, that is the four windings in the two pairs of windings are wound by one first winding 366, this being equivalent to four windings connected together in series. The winding directions of the first winding 364 and the second winding 365 are opposite. The motor bracket 363 is also provided with for example at least two support legs 369, and two ends of the first winding 366 are respectively connected to two of the support legs 369.

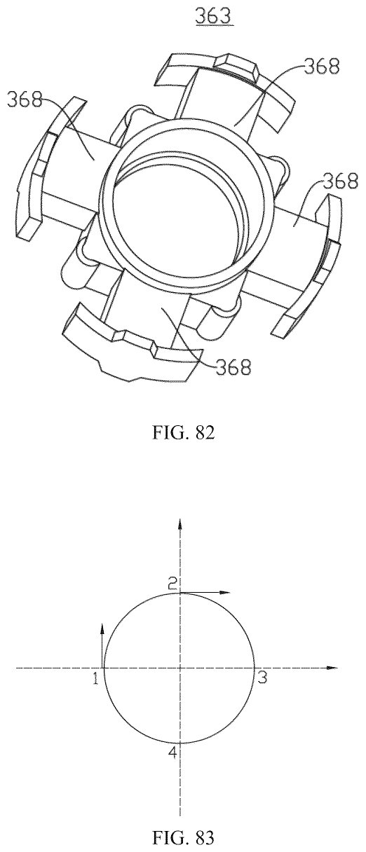

Taking FIG. 83 as an example for illustration, a pair of primary windings may comprise for example a first winding 364 and a second winding 365, and a pair of secondary windings may comprise for example a first winding 364 and a second winding 365, and Position 1 is the first winding 364 of a pair of secondary windings, and Position 2 is the first winding 364 of a pair of primary windings, and Position 3 is the second winding 365 of a pair of secondary windings, and Position 4 is the second winding 365 of a pair of primary windings. The winding direction of the first winding 364 of a pair of secondary windings can be for example counterclockwise, and the winding direction of the second winding 365 of a pair of secondary windings is thus clockwise; and the winding direction of the first winding 364 of a pair of primary windings is clockwise and the winding direction of the second winding 365 of a pair of primary windings is counterclockwise; it can also be that the winding direction of the first winding 364 of a pair of secondary windings is clockwise and the winding direction of the second winding 365 of a pair of secondary windings is counterclockwise; and the winding direction of the first winding 364 of a pair of primary windings is counterclockwise and the winding direction of the second winding 365 of a pair of primary windings is clockwise; of course, the winding directions of the first winding 364 and the second winding 365 can be adjusted according to the different positions of the stator 362. When the electric motor 36 is inputted with a power supply, the input current is, for example, a single-phase sinusoidal current, and a pair of primary windings will generate an alternating pulsating magnetic field. The strength of the alternating pulsating magnetic field changes with time as the sinusoidal current changes. Taking the first winding 364 of a pair of primary windings at Position 2 as an example, the direction of its magnetic field is, for example, from Position 1 to Position 3. The control chip 37 shifts the phase of the inputted single-phase sinusoidal current and applies the phase-shifted current to a pair of secondary windings. Taking the first winding 364 of a pair of secondary windings at Position 1 as an example, the direction of its magnetic field is, for example, from Position 4 to Position 2. After the electric motor 36 is inputted with the power supply, a portion of the single-phase sinusoidal current acts on a pair of primary windings for power supplying, and the other portion is controlled by the control chip 37 to control to do phase shift for then acting on a pair of secondary windings for power supplying. Since the phases of the single-phase sinusoidal current and the phase-shifted current are one in front of the other, the magnetic field forces generated by the corresponding pair of primary windings and the pair of secondary windings are also one in front of the other, so that the pair of primary windings and the pair of secondary windings can generate thrust in sequence, causing the rotor 361 to rotate and drive the impeller 30 to rotate. By having the control chip 37 controlling the magnetic field changes on the two pairs of windings to control the rotor 361 to drive the impeller 30 to rotate allows the fan assembly to have a higher and more stable speed, and the performance of the fan assembly is further improved.

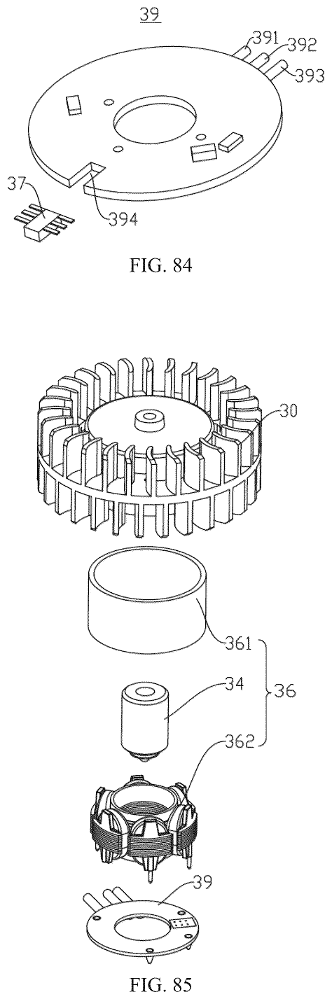

Referring to FIG. 84, the fan assembly may further comprise for example a circuit board 39, and the stator 362 is fixed on the circuit board 39, and the control chip 37 is arranged on the circuit board 39. The circuit board 39 may for example be provided with a current input line connecting terminal 391, a grounding terminal 392, and a pulse width modulation line connecting terminal 393. The current input line connecting terminal 391 is electrically connected to the power supply to receive input of current; the grounding terminal 392 is used for grounding; the pulse width modulation line connecting terminal 393 is electrically connected to the control chip 37. The control chip 37 can be specifically a Fengyi FT3206D sinusoidal wave drive chip. The control chip 37 may, for example, comprises a pulse width modulation signal output pin, and the control chip 37 may for example output the pulse width modulation signal through the PWM signal output pin to the pulse width modulation line connecting terminal 393, wherein the pulse width modulation signal refers to PWM signal. The control chip 37 can output PWM signals with different duty ratios, which may achieve an adjustment from 1% to 100%, thereby achieving 100 positions for rotation speed of the electric motor 36. For example, the fan assembly may also be provided with a speed adjustment switch, and the speed adjustment switch is electrically connected to the control chip 37. The control chip 37 can output PWM signals with different duty cycles in response to the speed adjustment switch to adjust the speed of the fan assembly. Steps of airflow speed adjustment of the fan assembly can reach 1%, achieving a stepless speed regulation effect, making the speed regulation more flexible and more convenient to use.

The circuit board 39 may be provided with for example a mounting trough 394, and the control chip 37 is disposed in the mounting trough 394. In this way, the thickness of the circuit board is reduced, making the assembly more compact and further reducing the size of the fan assembly. In addition, the distance between the control chip 37 and the two pairs of windings can be increased to reduce the influence of the control chip 37 on the magnetic field of the two pairs of windings. It can be understood that other components such as capacitors, resistors, and diodes may also be provided on the circuit board 39, which will not be described one by one in this embodiment.

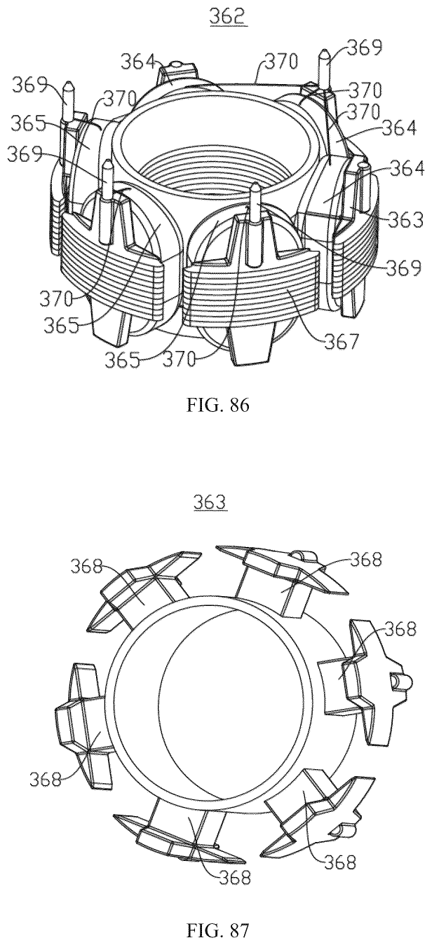

Referring to FIGS. 85, 86, and 87, in a specific way of implementation of a fifteenth embodiment of the application, compared with the above-described fourteenth embodiment, in the instant embodiment, the at least two pairs of windings may for example comprises three pairs of windings, and each pair of the windings comprises a first winding 364 and a second winding 365. The first winding 364 and the second winding 365 are arranged opposite to each other on the motor bracket 363. The stator 362 may comprise for example three second winding wires 370, and the motor bracket 363 is provided with six wire winding portions 368. The second winding wire 370 may be, for example, enameled wires. The silicon steel sheets 367 are connected to the motor bracket 363. A portion of the silicon steel sheets 367 may be, for example, wrapped in the six wire winding portions 368 of the motor bracket 363. Each of the second winding wires 370 is wound around two of the wire winding portions 368 that are opposite to each other to form a pair of windings. The winding directions of the first winding 364 and the second winding 365 in each pair of windings are opposite. The winding directions of three adjacent first windings 364 are the same, and the winding directions of three adjacent second windings 365 are the same. The motor bracket 363 may be provided with for example four support legs 369. Ends of the three second winding wires 370 are connected together, such as connecting the ends of the three second winding wires 370 to the same support leg 369, and the other ends of the three second winding wires 370 being respectively connected to the other three support legs 369. In the instant embodiment, correspondingly, the rotor 361 is provided with two pairs of magnetic poles. Of course, the rotor 361 may also be provided with one pair of magnetic poles or three pairs of magnetic poles. The rotor 361 being provided with two pair of magnetic poles is the best solution. When the rotor 361 is provided with two pairs of magnetic poles, the stator 362 and the rotor 361 can cooperate better to make the rotation of the rotor 361 more stable, and avoid the rotation jam of the rotor 361 resulting from the number of magnetic poles of the rotor 361 being large.

Three pairs of windings can be inputted with a three-phase AC power supply, for example. Since the three-phase AC power supply has a phase difference of 120°, when the three pairs of windings are inputted with the three-phase AC power supply, the three pairs of windings generate a circular rotating magnetic field to drive the rotor 361 to rotate. In this way, the electric motor 36 of the fan assembly can be started directly, and the starting current is small. The impeller 30 has a stronger rotational inertia than a single-phase motor, and the electric motor 36 rotates more smoothly. Secondly, the electric motor 36 has a simple structure, and there is no need to set up components such as starting capacitors and centrifugal switches, and the rotating magnetic field is directly generated by three-phase AC induction; in addition, the electric motor 36 has a large operating force, and the fan assembly rotates quickly and smoothly, and the noise is small; furthermore, the electric motor 36 is relatively energy-saving, which makes the fan assembly relatively energy-saving and improves the endurance of the fan assembly.

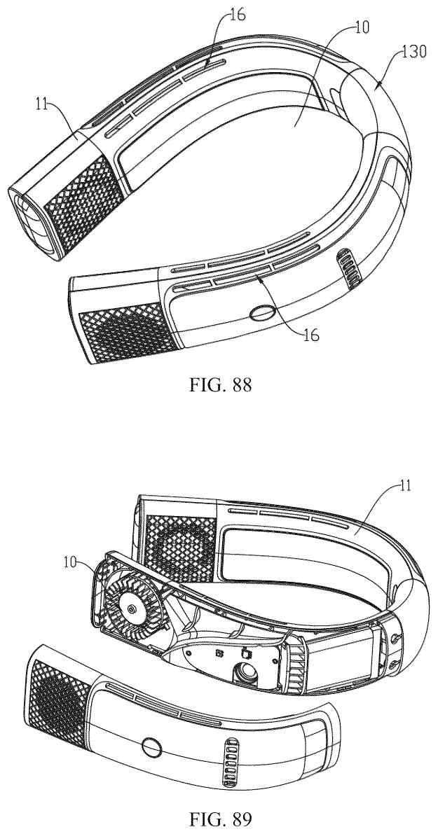

Referring to FIGS. 88 and 89, a sixteenth embodiment of the application further provides a portable temperature regulation device. The portable temperature regulation device can be a neck-mounted fan, a waist-mounted fan, a head-mounted fan, and a handheld fan. In the following, a neck-mounted fan is taken as an example for the portable temperature regulation device. The portable temperature regulation device may comprise for example a main body 11 and the fan assembly according to the above embodiment. The fan assembly is arranged in the main body 11. The main body 11 encloses and forms a wear space 10. The main body 11 is provided with for example a wearing portion 130, and the wearing portion 130 can be for example worn on the neck of the user. The main body 11 is also provided with an air outlet opening 16, and the air outlet opening 16 may for example blow the airflow blown out of the fan assembly toward the cheeks of the user. Of course, this is only an example, and the instant embodiment is not limited thereto.

As can be seen from the above, the above technical features of the application can have one or more of the following beneficial effects: the electric motor 36 in the fan assembly is arranged to include the stator 362 and the rotor 361 rotatable relative to the stator 362, and the stator 362 comprises the at least two pairs of windings, and the rotor 361 is arranged around the periphery of the at least two pairs of windings and is connected to the impeller 30, that is the electric motor 36 in the fan assembly is an outer rotor motor, which has the advantages of large moment of inertia, good heat dissipation, and saving winding wire of the winding. Load components, such as the impeller 30, can be directly connected to the rotor 361 to meet the small volume installation size requirements of the entire device, so that the electric motor 36 of the fan assembly has a simple structure, and is small in size and weight, making the fan assembly more suitable for portable temperature regulation devices. In addition, the control chip 37 is arranged to connect to the at least two pairs of windings on the stator 362, allowing the control chip 37 to control the rotor 361 to drive the impeller 30 to rotate by controlling the magnetic field changes on the at least two pairs of windings, so that the control chip 37 may better control the rotation of the electric motor 36, further improving the rotation effect of the fan assembly.

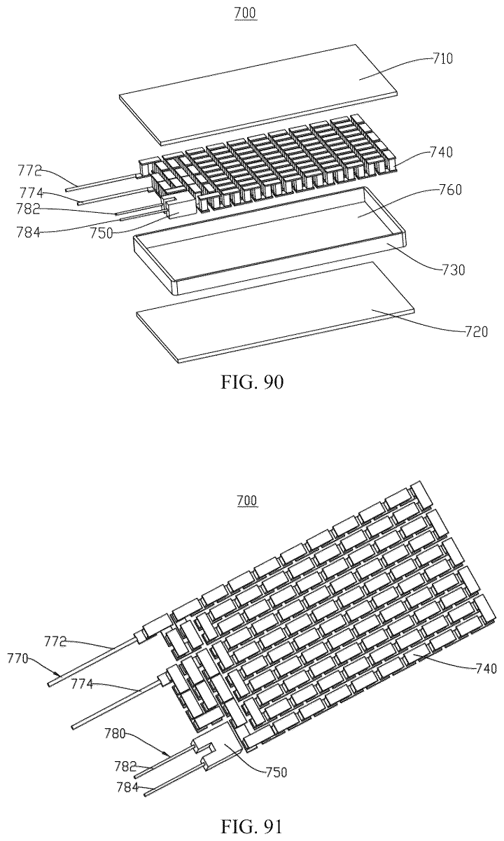

Referring to FIG. 90, a seventeenth embodiment of the application provides a novel semiconductor cooler device 700, which can be used as for example the temperature regulation member of any one of the first embodiment to the eighth embodiment described above. Specifically, the semiconductor cooler device 700 comprises for example: a first substrate 710, a second substrate 720, an encapsulating member 730, a plurality of thermocouple pairs 740, and a temperature detection unit 750.

In the above, the encapsulating member 730 is arranged between the first substrate 710 and the second substrate 720, and the encapsulating member 730 together with the first substrate 710 and the second substrate 720 enclose and form a receptacle space 760. The plurality of thermocouple pairs 740 and the temperature detection unit 750 are for example both arranged between the first substrate 710 and the second substrate 720 and located in the receptacle space 760.

Further, the semiconductor cooler device 700 further comprise for example a first wire connecting terminal 770 and a second wire connecting terminal 780. The first wire connecting terminal 770 is electrically connected to the plurality of thermocouple pairs 740 and extends to the outside of the receptacle space 760. The second wire connecting terminal 780 is electrically connected to the temperature detection unit 750 and extends to the outside of the receptacle space 760. Specifically, the first wire connecting terminal 770 comprises for example a first connection wire 772 and a second connection wire 774, and the second wire connecting terminal 780 comprises for example a third connection wire 782 and a fourth connection wire 784. For example, the first connection wire 772 and the second connection wire 774 are for example power lines, and the third connection wire 782 and the fourth connection wire 784 are for example signal lines. Among them, the first connection wire 772 and the second connection wire 774 are electrically connected to the plurality of thermocouple pairs 740 and extend to the outside of the receptacle space 760 for being electrically connected to an external circuit; and the third connection wire 782 and the fourth connection wire 784 are electrically connected to the temperature detection unit 750 and extend to the outside of the receptacle space 760 for being electrically connected to an external circuit. For easy distinction of the first wire connecting terminal 770 and the second wire connecting terminal 780, the first wire connecting terminal 770 and the second wire connecting terminal 780 use for example wires of different thicknesses or wires of different colors. For example, the first connection wire 772 and the second connection wire 774 use thin wires, while the third connection wire 782 and the fourth connection wire 784 use thick wires; or alternatively, the first connection wire 772 and the second connection wire 774 respectively use a white wire and a blue wire, and the third connection wire 782 and the fourth connection wire 784 respectively use a black wire and a red wire. In other embodiments, the first wire connecting terminal 770 and the second wire connecting terminal 780 may also adopt other forms of wiring connection, as long as the plurality of thermocouple pairs 740 and the temperature detection unit 750 can be connected to the external circuits respectively.

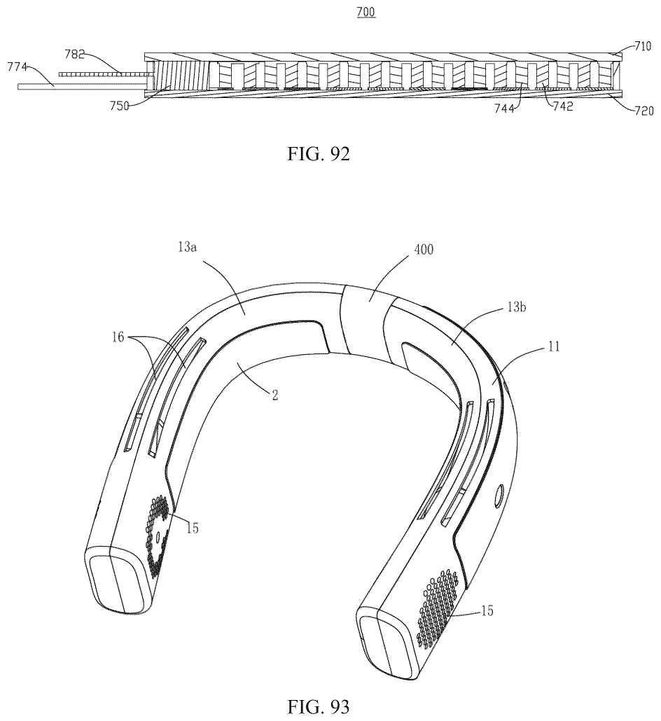

Continuing from the above, as shown in FIG. 92, each thermocouple pair 740 comprises for example a first type semiconductor thermocouple 742 and a second type semiconductor thermocouple 744 connected in series. The first type semiconductor thermocouple 742 is, for example, an N-type semiconductor thermocouple, and the second type semiconductor thermocouple 744 is, for example, a P-type semiconductor thermocouple, and vice versa. Because the carriers of the N-type semiconductor thermocouple are electrons and the carriers of the P-type semiconductor thermocouple are holes, and the current directions of different types of semiconductor thermocouples are opposite, the electrons of the N-type semiconductor thermocouple and the holes of the P-type semiconductor thermocouple flow in the same direction, wherein the carriers of the semiconductor thermocouple become the medium for heat transfer, and the external DC power supply provides the energy required for the electron flow. After the power supply is provided, the electrons start from the negative terminal (−), first passing through the P-type semiconductor thermocouple and absorbing heat here, and then releasing the heat at the N-type semiconductor thermocouple. Every time they passing through a pair of N-type and P-type semiconductor thermocouples, heat is transferred from one end to the other end. Because of this active heat pumping, a temperature difference is caused, forming cold and hot ends. When the current direction is opposite, the direction of heat transfer is also opposite. This principle can be used for temperature control. The temperature detection unit 750 is used to detect the operating temperature of the semiconductor cooler device 700 to obtain a detection result, and outputs the detection result to an external circuit.

For example, the external circuit mentioned above is, for example, an external control circuit. The plurality of thermocouple pairs 740 are electrically connected to the external control circuit through the first connection wire 772 and the second connection wire 774 to achieve cold-end cooling and hot-end heating. The temperature detection unit 750 is electrically connected to the external control circuit through the third connection wire 782 and the fourth connection wire 784 to detect the operating temperature of the semiconductor cooler device 700 and transmit the detection result to the external control circuit. When the operating temperature exceeds a preset value, a controller of the external control circuit can timely adjust the heat dissipation efficiency of the semiconductor cooler device 10 or directly shut down the semiconductor cooler device 700 to protect the semiconductor cooler device 700 from damage.

In the embodiment of the application, the first substrate 710, the second substrate 720, and the encapsulating member 730 are arranged to enclose and form the receptacle space 760, so that the receptacle space 760 is isolated from the external environment, wherein the encapsulating member 730 provides functions of moisture-proof and heat-insulating, and the temperature detection unit 750 is integrated and arranged inside the receptacle space 760, that is the semiconductor cooler device 700 itself is provided with the temperature detection unit 750 inside and there is no need to separately set up a temperature detection unit, and this can not only avoid the situation in the prior art that the temperature detection unit 750 is relatively detached from the semiconductor cooler device 700 due to factors, such as external forces, but also avoid the influence of the ambient temperature on the temperature detection process, so as to more accurately detect the operating temperature of the semiconductor cooler device 700. Further, the temperature detection unit 750 is integrated and arranged inside the semiconductor cooler device 700, and the temperature detected by the temperature detection unit 750 is the interior operating temperature of the semiconductor cooler device 700, rather than reflecting the operating temperature at the attachment site of the outer surface of the semiconductor cooler device 700, so that the temperature change during the operation of the semiconductor cooler device 700 can be more accurately reflected, thereby further improving the operating temperature detection accuracy of the semiconductor cooler device 700.

The structure of the semiconductor cooler device 700 will further described with reference to FIGS. 90-92.

Further referring to FIG. 90, the first substrate 710 and the second substrate 720 are arranged opposite to each other, for example. In an example, the first substrate 710 and the second substrate 720 are arranged, for example, parallel to each other. The first substrate 710 can be for example a cold-side substrate, and the second substrate 720 can be for example a hot-side substrate, and vice versa. The first substrate 710 and the second substrate 720 can be fixedly connected by for example the encapsulating member 730, and the encapsulating member 730 is for example filled in the peripheral edge between the first substrate 710 and the second substrate 720 to maintain the stability of the connection between the two. In an example, the encapsulating member 730 may be selected as rubber, and the rubber, after cured, is an elastic solid presenting milky white. The purpose of curing is to isolate the plurality of thermocouple pairs 740 and the temperature detection unit 750 from the external environment, providing effects of moisture-proof and heat insulation. Further, the first substrate 710 and the second substrate 720 can be for example ceramic substrates, such as substrates of materials of Al₂O₃ (aluminum oxide), BeO (bismuth oxide), and AlN (aluminum nitride) so as to not only possess excellent thermal conductivity, but also having excellent electrical insulation properties, making the semiconductor cooler device 700 fulfill the operation performance while simplifying the structure. In a specific embodiment, one or two of the first substrate 710 and the second substrate 720 can be for example metal substrate, wherein the inside surface of the metal substrate is provided with an insulation layer. In an example, the metal substrate can be selected as an aluminum substrate, a copper substrate, or other metal conductors. The location on the inside surfaces of the first substrate 710 and the second substrate 720 in contact with the plurality of thermocouple pairs 740 is isolated by the insulation layer so as to, on one hand, ensure the electrical isolation of the current flowing into the plurality of thermocouple pairs 740 from the first substrate 710 and the second substrate 720, and on the other hand, make full use of the better thermal conductivity of the metal materials of the first substrate 710 and the second substrate 720 to conduct away the cold and heat generated by the plurality of thermocouple pairs 740.

Continuing from the above, the plurality of thermocouple pairs 740 can be for example fixedly clamped between the first substrate 710 and the second substrate 720 and are sequentially connected in series between the first connection wire 772 and the second connection wire 774, such as that shown in FIG. 91. The number of the plurality of thermocouple pairs 740 is preferably 100-120, preferably 103 or 105, so that the semiconductor cooler device 700, when used in a portable device, may supply sufficient cold or heat to enhance the user's experience. Referring to FIG. 92, each thermocouple pair 740 comprises for example a first type semiconductor thermocouple 742 and a second type semiconductor thermocouple 744 connected in series. Each one of the first type semiconductor thermocouple and the second type semiconductor thermocouple has a height H1 of 1.0 mm-2.0 mm in the distance direction of the first substrate and the second substrate. The height H1 is preferably 1.7 mm, which is conducive to the thinning of the semiconductor cooler device 700, so as to be more suitable for portable products.

Further, the length and the width of the first type semiconductor thermocouple 742 and the second type semiconductor thermocouple 744 of each thermocouple pair 740 are both preferably 1.0 mm. The distance between the first type semiconductor thermocouple 742 and the second type semiconductor thermocouple 744 of each thermocouple pair 740 is 0.5 mm-1.2 mm. Referring to FIG. 92, a first distance D1 is between the first type semiconductor thermocouple 742 and the second type semiconductor thermocouple 744 of a first thermocouple pair of two adjacent thermocouple pairs 740, and a second distance D2 is between the first type semiconductor thermocouple 742 and the second type semiconductor thermocouple 744 of a second thermocouple pair of the two adjacent thermocouple pairs 740, and the second distance D2 is different from the first distance D1. For example, the first distance D1 can be for example 0.5 mm-0.7 mm, and the first distance D1 is preferably 0.6 mm, and the second distance D2 can be for example 0.8 mm-1.2 mm, and the second distance D2 is preferably 1.0 mm. Here, the first distance D1 and the second distance D2 being set to be different and further designing the numerical ranges of the first distance D1 and the second distance D2 provide the semiconductor cooler device 700 with a better cooling or heating effect.

Further, referring to FIGS. 90 and 91 again, the temperature detection unit 750 is disposed in an edge region between the first substrate 710 and the second substrate 720. The edge region is located outside a disposition region of the plurality of thermocouple pairs 740. The disposition region of the plurality of thermocouple pairs 740 refers to an area formed as being spreading outward from the center between the first substrate 710 and the second substrate 720. In this way, on the one hand, adding the temperature detection unit 750 inside the semiconductor cooler device 700 does not affect the disposition of the plurality of thermocouple pairs 740, and, on the other hand, it is also convenient to lead out fry the third connection wire 782 and the fourth connection wire 784 of the temperature detection unit 750, thereby simplifying the manufacturing operation of the semiconductor cooler device 700.

Further, the temperature detection unit 750 is, for example, attached to the inner surface of the first substrate 710, thereby simplifying the assembly operation of assembling the temperature detection unit 750 in the interior of the semiconductor cooler device 700. For example, the temperature detection unit 750 comprises for example a thermistor. The temperature inside the semiconductor cooler device 700 detected by the temperature detection unit 750 is the operating temperature of the semiconductor cooler device 700. An external circuit can optimize a control curve of the semiconductor cooler device 700 according to the detection result of the temperature detection unit 750, thereby improving the accuracy of temperature regulation of the semiconductor cooler device 700, and when the operating temperature inside the semiconductor cooler device 700 becomes excessively high, protection control of the semiconductor cooler device 700 can be started in time, thereby extending the service life of the semiconductor cooler device 700. This can also avoid the safety risks that may be caused by overheating of the semiconductor cooler device 700.

In summary, the seventeenth embodiment of the application provides a novel semiconductor cooler device, which uses the first substrate, the second substrate, and the encapsulating member to enclose and form the receptacle space, and makes the receptacle space isolated from the external environment, wherein the encapsulating member provides effects of moisture-proof and heat-insulating, and the temperature detection unit is integrated and arranged inside the receptacle space, that is the semiconductor cooler device itself is provided with the temperature detection unit inside and there is no need to separately set up a temperature detection unit, and this can not only avoid the situation in the prior art that the temperature detection unit is relatively detached from the semiconductor cooler device due to factors, such as external forces, but also avoid the influence of the ambient temperature on the temperature detection process, so as to more accurately detect the operating temperature of the semiconductor cooler device. Further, the temperature detection unit is arranged inside the semiconductor cooler device, and the temperature detected by the temperature detection unit is the interior operating temperature of the semiconductor cooler device, rather than reflecting the operating temperature at the attachment site of the outer surface of the semiconductor cooler device, so that the temperature change during the operation of the semiconductor cooler device can be more accurately reflected, thereby further improving the operating temperature detection accuracy of the semiconductor cooler device. Further, the first distance and the second distance of the first type semiconductor thermocouple and the second type semiconductor thermocouple are set to be different, so that the semiconductor cooler device can have a better cooling or heating effect. In addition, by designing the number of the thermocouple pairs, sufficient cold or heat can be provided when used in portable devices to enhance the user's experience. Further, by designing the heights of the first type semiconductor thermocouple and the second type semiconductor thermocouple, it is conducive to the thinning of the semiconductor cooler device, making it more suitable for portable products.

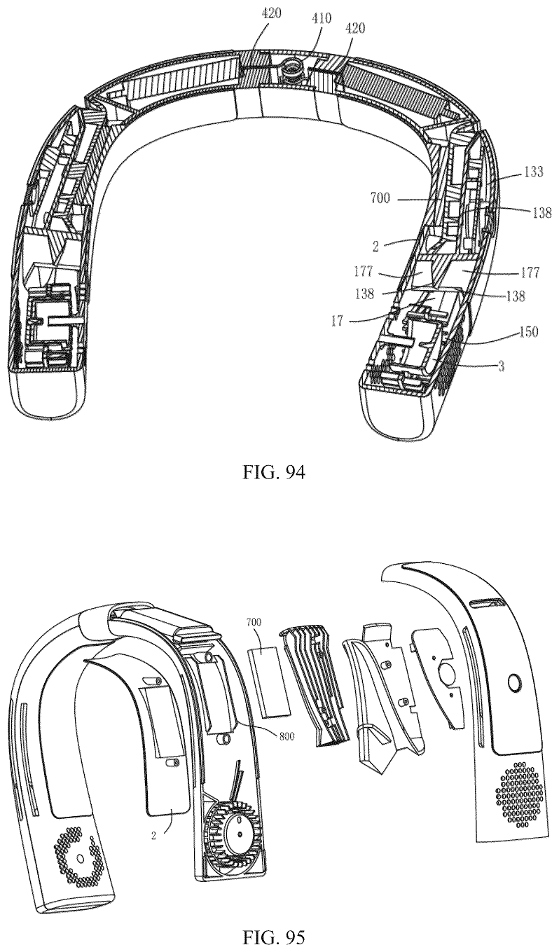

Referring to FIGS. 93 and 94, an eighteenth embodiment of the application provides a portable temperature regulation device, which comprises a main body 11, a semiconductor cooler device 700 of the previously described seventeenth embodiment arranged in the main body 11, and a temperature conducting member 2 arranged in the main body 11. The temperature conducting member 2 is in thermal conduction connection with the first substrate 710 of the semiconductor cooler device 700. The portable temperature regulation device can a temperature regulation product wearable on various parts of a user's body, and the main body 11 correspondingly refers to a structure that stably wears the portable temperature regulation device on the part of the user's body. For example, the portable temperature regulation device is a temperature regulation device wearable on the user's wrist, wherein the main body 11 may be a wristband around the user's wrist; the portable temperature regulation device may be a temperature regulation device wearable on the user's waist, wherein the main body 11 may be a fastening belt; the portable temperature regulation device may be a temperature regulation device wearable on the user's neck, wherein the main body 11 may be a neck-worn frame arranged around the user's neck. In the instant embodiment, in order to facilitate understanding, a neck-mounted temperature regulation device is taken as an example for illustrating the portable temperature regulation device.

In some embodiments, the main body 11 comprises a first arm portion 13a, a second arm portion 13b, and a connecting structure 400 connecting the first arm portion 13a and the second arm portion 13b in a curvable manner. The first arm portion 13a and the second arm portion 13b are each provided, in an interior thereof, with the semiconductor cooler device 700. The first arm portion 13a and the second arm portion 13b are each provided with the temperature conducting member 2 on an inner side thereof.

In some embodiments, referring to FIG. 95, the first arm portion 13a and the second arm portion 13b are each provided with a positioning structure 800 in the interior thereof for fixing the semiconductor cooler device 700. The inner side refers to the side of the portable temperature regulation device close to the skin surface of the user's body when worn.

In some embodiments, further referring to FIG. 94, the connecting structure 400 comprises an elastic member 410 and two connecting members 420. The two connecting members 420 are connected in a manner of being rotatable relative to each other, and the connecting members 420 are respectively connected to the first arm portion 13a and the second arm portion 13b. Two ends of the elastic member 410 respectively contact and abut against the two connecting members 420 in order to apply an elastic force through the connecting members 420 to the first arm portion 13a and the second arm portion 13b for rotating toward the inner side. Thus, when the neck-mounted temperature regulation device is worn on the user's neck, the temperature conducting members 2 located on the inner sides of the first arm portion 13a and the second arm portion 13b may maintain contacting with the skin of the neck.

In some embodiments, further referring to FIGS. 93 and 94, the first arm portion 13a and the second arm portion 13b are each provided with an air inlet opening 15 and an air outlet opening 16, and the first arm portion 13a and the second arm portion 13b are each provided, in the interior thereof, with an accommodation compartment 150 in communication with the air inlet opening 15 and an air channel 17 communicating the accommodation compartment 150 with the air outlet opening 16. The first arm portion 13a and the second arm portion 13b may each be further provided with a fan 3 received in the accommodation compartment 150. The air channel 17 can be divided into and thus form a plurality of sub air channels 177 by a partition member 138. The air outlet openings 16 on the first arm portion 13a and the second arm portion 13b may be formed as a plurality of sub air outlet openings corresponding to different sub air channels 177. In some embodiments, the interior spaces of the first arm portion 13a and the second arm portion 13b may include a receiving compartment 133 separated and formed by the partition member 138 and separated from the air channel 17. The external control circuit of the semiconductor cooler device 700 may be arranged in the receiving compartment 133. An airflow generated during the operation of the fan 3 mainly flow toward each of the sub air channels 177 and is divided and guided by the sub air channel 177 to flow out through the corresponding sub air outlet openings 16, in order to blow air to different parts of the human body for dissipating heat. A minor portion of the airflow flows into the receiving compartment 133 to dissipate heat from the circuit in order to ensure the stability of operation of the circuit.

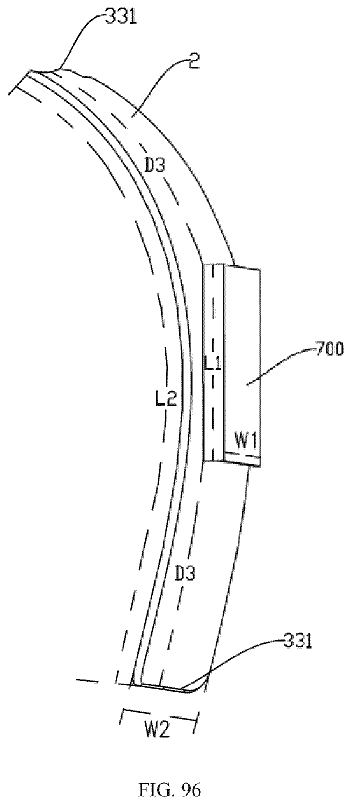

Further, in a specific embodiment, referring to FIG. 96, the length L1 of the semiconductor cooler device 700 is 30 mm-45 mm, and the width W1 of the semiconductor cooler device 700 is 15 mm-25 mm. The size of the semiconductor cooler device 700 is preferably 40 mm in length L1 and 20 mm in width W1. In this way, the semiconductor cooler device volume can be reduced as much as possible while ensuring the cooling/heating effect of the semiconductor cooler device 700, and the cooling/heating efficiency of the semiconductor cooler device 700 can be improved under the same volume, so that it can be applied to portable products having a smaller size.

In some embodiments, further referring to FIG. 96, the ratio of the length L1 of the semiconductor cooler device 700 to the length L2 of the temperature conducting member 2 is 0.25-0.5, and the ratio is preferably 0.3. The ratio of the width W1 of the semiconductor cooler device to the width W2 of the temperature conducting member is 0.5-0.75, and the ratio is preferably 0.6. Further, the temperature conducting member 2 has two opposite edges 331 in the length direction thereof. The distance D3 from the semiconductor cooler device 700 to any edge 331 in the length direction of the temperature conducting member 2 is 15 mm-30 mm. Here, through reasonable design of the dimensional relationship between the semiconductor cooler device 700 and the temperature conducting member 2, the conduction efficiency and conduction uniformity of the temperature conducting member 2 can be ensured.

In summary, the seventeenth embodiment of the application provides a portable temperature regulation device that has a more accurate temperature monitoring of the semiconductor cooler device, making the performance of a product for temperature control better; and through reasonable design of the dimensional relationship between the semiconductor cooler device and the temperature conducting member, the conduction efficiency and conduction uniformity of the temperature conducting member can be ensured.

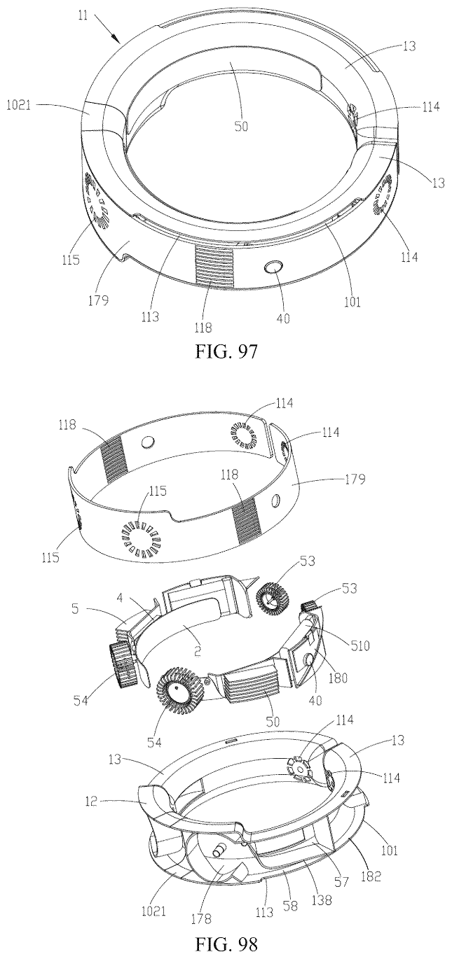

Referring to FIGS. 97-99, a nineteenth embodiment of the application provides a portable temperature regulation device wearable on a human body, such as being worn on the neck or waist of a human body to achieve a cooling or heating effect. In the following, being worn on the neck is taken as an example for illustration.

Comparing the portable temperature regulation device of the instant embodiment with the portable temperature regulation device of the first embodiment, in the instant embodiment, the portable temperature regulation device comprises a main body 11 and a plurality of temperature regulation units. The main body 11 is wearable on the neck of a user, and the plurality of temperature regulation units are individually mounted on the main body 11 for temperature regulation so as to achieve an effect of cooling or heating. The main body 11 is formed in the interior thereof with a chamber 178 for accommodating the temperature regulation units. It is understood that the temperature regulation units can be completely received in the chamber 178, or can be partly received in the chamber 178 and partly located outside the chamber 178.

The specific way of wearing the main body 11 on the neck is not limited, such as being worn from the user's down to directly put on the user's neck; or alternatively, the main body 11 is arranged to have two end portions thereof movable relative to each other so that the user can adjust the distance between the two ends of the main body 11 to directly put the portable temperature regulation device on the neck in a sideway insertion manner. In the instant embodiment, the two ends of the main body 11 can move relative to each other for being put on the neck through sideway insertion, so that the portable temperature regulation device is not affected by the head during wearing, making it possible to reduce the size of the portable temperature regulation device and also allow the portable temperature regulation device to be easily worn and taken off.

The specific shape of the main body 11 is not limited and can be for example a U-shape, a C-shape, or a circular ring. In the instant embodiment, the main body 11 is of a circular ring structure, and the circular ring can be an open or closed circular ring. Comparing the circular ring with the U-shape and C-shape, the distance between two ends of the circular ring is relatively small or even not existing, so that the portable temperature regulation device, after worn on the neck, is not easy to fall off, thereby reducing the risk of the portable temperature regulation device falling of the neck during exercise of the user.

The main body 11 comprises two arm portions 13 and a connecting member 1021. Each arm portion 13 is provided with a temperature regulation unit. The two arm portions 13 are respectively connected to two ends of the connecting member 1021. Each arm portion 13 has the chamber 178 for accommodating the temperature regulation unit.

The specific number of temperature regulation units mounted in each of the arm portions 13 is not limited, and can be one or multiple. In the instant embodiment, the number of the temperature regulation units is two, and each of the arm portions 13 is provided with one temperature regulation unit.

In some embodiments, the other end of the arm portion 13 away from the connecting member 1021 is movable relative to each other. Thus, during the course of being put on or taken off from the user, the end portions of the two arm portions 13 away from the connecting member 1021 are first moved relative to each other to have the gap between the two end portions wider than the width of the neck so as to allow the neck to pass through the gap between the two end portions.

The specific way for achieving relative movement between the ends of the two arm portions 13 away from the connecting member 1021 is not limited. For example, it is feasible to have the two arm portions 13 each rotatably connected to the connecting member 1021, and the distance between the two end portions can be achieved by means of rotating the two arm portions 13, or alternatively, it is also feasible to make the connecting member 1021 elastic and thus deformable, and the two arm portions 13 may achieve relative movement by compressing or stretching the connecting member 1021. In the instant embodiment, the connecting member 1021 is capable of deformation.

The specific type of the connecting member 1021 is not limited, and can be for example a silicone member that is deformable, or can be an extendable/retractable flexible tube. In the instant embodiment, the connecting member 1021 is a silicone member, and the silicone member is elastic and is deformable. When the two arm portions 13 are subjected to an outward force, the ends of the two arm portions 13 away from the connecting member 1021 move away from each other and thus stretch the silicone member.

The main body 11 further comprises a fixing plate 179. The two arm portions 13 and the connecting member 1021 are all connected to the fixing plate 179.

In some embodiments, the fixing plate 179 has an elastic restoration function. When the user drives the ends of the two arm portions 13 away from the connecting member 1021 to move away from each other, the fixing plate 179 deforms to move with the two arm portions 13. When the user releases the two arm portions 13, the ends of the two arm portions 13 away from the connecting member 1021 are moved toward each other under the action of the fixing plate 179 to have the main body 11 finally restore back to the original state, fulfilling an effect of automatic restoration, that is restoring back to the circular ring shape to prevent the portable temperature regulation device from falling off the neck.

The specific material of the fixing plate 179 is not limited, and can be for example an elastic metal plate or an elastic plastic plate, as long as an elastic restoration effect may be achieved. In the instant embodiment, the fixing plate 179 is an elastic metal plate.

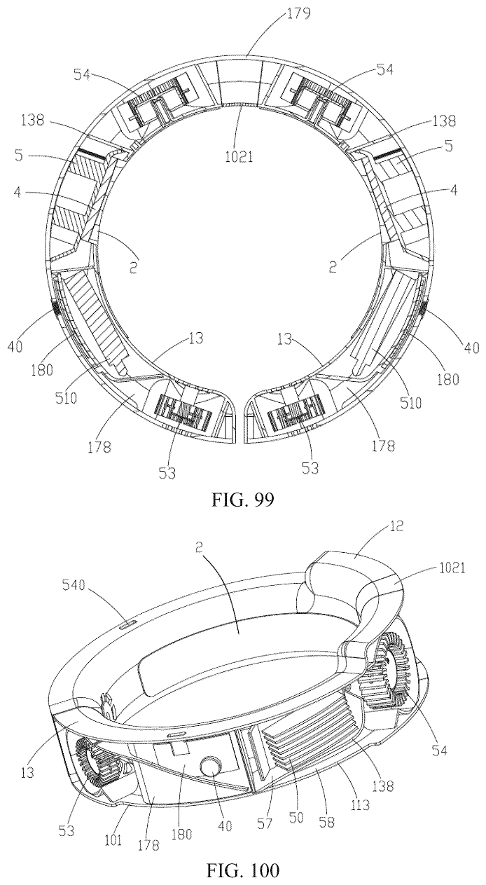

Referring to FIGS. 99 and 100, each temperature regulation unit comprises a first fan 53, and the first fan 53 is located in the chamber 178. The main body 11 is provided with a first air inlet opening 114 and a first air outlet opening 101. The first air inlet opening 114 and the first air outlet opening 101 are both in communication with the chamber 178. During the operation of the first fan 53, air outside of the portable temperature regulation device is drawn into the chamber 178 through the first air inlet opening 114 and is then blown out from the first air outlet opening 101 as being driven by the first fan 53 so as to blow toward the user to provide an effect of air blowing and cooling.

The specific type of the first fan 53 is not limited, and can be for example an axial flow fan or a centrifugal fan. In the instant embodiment, the first fan 53 is a centrifugal fan. The first air inlet opening 114 is arranged on a radial side portion of the main body 11 (that is an inner side close to the user's neck or an outer side away from the user's neck), and the first air outlet opening 101 is arranged on an axial side portion of the main body 11. Specifically, the fixing plate 179 and the arm portions 13 are each provided with the first air inlet opening 114 on the side thereof away from the fixing plate 179. The first air outlet opening 101 is arranged on the axial side of the arm portions 13. Arranging two first air inlet openings 114 may increase the air intake volume, so as to have more air passing through the first air inlet opening 114 to get into the chamber 178 to enhance the air blowing and cooling effect of the first fan 53.

The specific shapes of the first air outlet opening 101 and the first air inlet opening 114 are not limited, and can be for example through holes formed in the main body 11 or through slots formed in the main body 11.

Each temperature regulation unit further comprises a second fan 54 and a temperature regulation assembly 50. The second fan 54 is located in the chamber 178. The main body 11 is provided with a second air inlet opening 115, a second air outlet opening 113, and a heat dissipation opening 118 in communication with the chamber 178. A first air egress path 57 is formed between the second fan 54 and the heat dissipation opening 118, and a second air egress path 58 is formed between the second fan 54 and the second air outlet opening 113. The temperature regulation assembly 50 comprises a temperature regulation member 4 arranged in a first air egress path 57 and a temperature conducting member 2 connected to the temperature regulation member 4. The temperature conducting member 2 is at least partly located outside the chamber 178 for being easy to contact the user. Specifically, the temperature conducting member 2 is located on the inner side of the arm portion 13. During the operation of the second fan 54, air outside the portable temperature regulation device is drawn into the chamber 178 through the second air inlet opening 115 and is then driven by the second fan 54 to have a portion of the air enter the first air egress path 57, flowing through the temperature regulation member 4 of the temperature regulation assembly 50 to bring away heat from the temperature regulation member 43 and finally discharged through the heat dissipation opening 118, and a portion of the air enters the second air egress path 58 and is finally discharged through the second air outlet opening 113 to blow toward the user, achieving an effect of air blowing and cooling. The first fan 53 and the second fan 54 both have the effect of air blowing and cooling, increasing the air blowing and cooling range of the portable temperature regulation device. The temperature regulation member 4 can generate cold flow and hot flow. The temperature conducting member 2 is used to contact the user in order to transfer the cold flow or hot flow to the user to form contact cooling or heating, namely an effect of temperature regulation, for the user. To cool the user, cold flow is generated on the side of the temperature regulation member 4 connected to the temperature conducting member 2 and is transmitted through the temperature conducting member 2 to the user, while hot flow is generated on the side of the temperature regulation member 4 away from the temperature conducting member 2, and air inside the first air egress path 57 flows through the temperature regulation member 4 during movement toward the heat dissipation opening 118 and brings away the hot flow from the temperature regulation member 4 in order to ensure the cooling effect of the temperature regulation member 4. The second fan 54, while dissipating heat from the temperature regulation member 4, also achieves an effect of air blowing and cooling for the user to enhance the cooling effect of the multifunctional temperature regulation device.

The fixing plate 179 is provided with a first air inlet opening 114, a second air inlet opening 115, and a heat dissipation opening 118 corresponding to each temperature regulation unit. Specifically, the number of the temperature regulation units is two, and the two temperature regulation units are respectively arranged in the two arm portions 13. Each temperature regulation unit comprises a first fan 53 and a second fan 54. The fixing plate 179 is provided with a first air inlet opening 114 to correspond to each of the two first fan 53 and is provide with a second air inlet opening 115 and a heat dissipation opening 118 to correspond to each of the two second fan 54.

The specific way of arranging the fixing plate 179 is not limited, and can be for example located inside the main body 11 or can be located outside the main body 11. In the instant embodiment, the main body 11 encloses and forms a wearing cavity. The wearing cavity is located on the inner side of the main body 11. The main body 11 is worn on the neck of the user through the wearing cavity. One side of the two arm portions 13 and the connecting member 1021 away from the wearing cavity is formed with an installation opening 182 in communication with the chamber 178. The installation opening 182 communicates with the chamber 178 and the outside of the main body 11, that is the outer side of the arm portions 13 and the connecting member 1021 in the radial direction is arranged in an open form. The fixing plate 179 is embedded in the installation opening 182. By forming the installation opening 182 on the outer side of the main body 11 and having the fixing plate 179 embedded in the installation opening 182, the fixing plate 179 not only has an effect of restoration, but also forms an effect of covering the chamber 178 to protect internal components so as to be conducive to reducing the overall size and weight of the portable temperature regulation device. Specifically, the outside surface of the fixing plate 179 is flush with the outside surfaces of the arm portions 13 and the connecting member 1021 to reduce the abruptness of the fixing plate 179.

The specific type of the second fan 54 is not limited, and can be for example an axial flow fan, or can be a centrifugal fan. In the instant embodiment, the second fan 54 is a centrifugal fan. The second air inlet opening 115 and the heat dissipation opening 118 are arranged in a radial side portion of the main body 11 (namely inner side or outer side), and the second air outlet opening 113 is arranged on an axial side portion of the main body 11. Specifically, the second air inlet opening 115 and the heat dissipation opening 118 are both arranged on the fixing plate 179, and the second air outlet opening 113 is arranged on an axial side of the arm portions 13 and is located on the same side of the main body 11 as the first air outlet opening 101. When the portable temperature regulation device is worn on the neck, the fixing plate 179 is located on the side away from the neck, and thus, arranging the heat dissipation opening 118 on the fixing plate 179 can prevent the hot air discharged from the heat dissipation opening 118 from blowing toward the user to affect the cooling effect.

The specific shapes of the second air outlet opening 113, the second air inlet opening 115, and the heat dissipation opening 118 are not limited, and can be example through holes formed in the main body 11 or can be through slots formed in the main body 11.

In some embodiments, filter nets can be provided at the first air inlet opening 114 and the second air inlet opening 115 to provide a filtering effect to the air entering the chamber 178, preventing dust in the external air from entering the chamber 178.

As shown in FIG. 100, in the instant embodiment, a partition member 138 is arranged in the chamber 178 of the main body 11. The partition member 138 separates the chamber 178 into a first air egress path 57 and a second air egress path 58. Specifically, the first air egress path 57 and the second air egress path 58 are located on the same side of the second fan 54 and extend along the length direction of the arm portions 13. The partition member 138 separates the first air egress path 57 and the second air egress path 58, so that the air in the two air egress paths does not circulate with each other, thereby preventing the hot air in the first air egress path 57 that is formed through cooling the temperature regulation member 4 from flowing into the second air egress path 58 to affect the air blowing and cooling effect of the second fan 54. In other embodiments, there can be no partition member 138 arranged in the chamber 178. For example, when the first air egress path 57 and the second air egress path 58 are not on the same side of the second fan 54, the flowing directions of air flowing in the first air egress path 57 and the second air egress path 58 are different, and the air in the two air egress paths does not interfere with each other, so that there is no need to arrange a partition member 138 to separate them.

In the embodiment illustrated in FIG. 99, the first fan 53 and the second fan 54 that are located in the same arm portion 13 are respectively located at two ends of the arm portions 13, that is one of the first fan 53 and the second fan 54 is arranged at one end of the arm portion 13 close to the connecting member 1021, and the other one is arranged on one end of the arm portion 13 away from the connecting member 1021. The first air outlet opening 101 extends in a direction from the first fan 53 toward the second fan 54 along the length direction of the arm portion 13 (that is the circumferential direction of the main body 11 in the instant embodiment), and the second air outlet opening 113 extends from the second fan 54 toward the first fan 53 along the length direction of the arm portion 13 (that is the circumferential direction of the main body 11 in the instant embodiment). By arranging the first fan 53 and the second fan 54 at the two ends of the arm portion 13 respectively, the lengths of the first air outlet opening 101 and the second air outlet opening 113 in the length direction of the arm portion 13 can be made longer, so that the first fan 53 and the second fan 54 can provide to more areas of the user neck with an air blowing and cooling effect.

In the twentieth embodiment shown in FIG. 102, comparing with the nineteenth embodiment described previously, in the instant embodiment, the first fan 53 and the second fan 54 that are located in the same arm portion 13 are arranged in the middle region of the arm portion 13, and the second fan 54 is closer to the connecting member 1021 as compared with the first fan 53, and the air outlet directions of the first fan 53 and the second fan 54 are opposite. The first air outlet opening 101 extends in a direction away from the connecting member 1021 along the length direction of the arm portions 13, and the second air outlet opening 113 extends in a direction from the second fan 54 toward the connecting member 1021 along the length direction of the arm portions 13. The first air egress path 57, the second air egress path 58, and the partition member 138 are located between the second fan 54 and the connecting member 1021.

As shown in FIG. 100, in the instant embodiment, the second fan 54 is arranged at one end of the arm portion 13 close to the connecting member 1021. One end of the arm portion 13 close to the connecting member 1021 protrudes outward along the width direction of the arm portions 13 to form a protrusion portion 12 for accommodating the second fan 54. In the instant embodiment, in the width direction of the arm portion 13, the size of the second fan 54 is greater than the size of the first fan 53. When the portable temperature regulation device is worn on the neck, the connecting member 1021 and its surrounding area automatically are at the position of the back of the user's neck, and under the action of gravity, the inner side of the connecting member 1021 and its surrounding area automatically fit the back of the user's neck, so that is it inconvenient to open the second air inlet opening 115 in the end of the arm portion 13 close to the connecting member 1021, this limiting the air intake volume of the second fan 54. By arranging the protrusion portion 12 at one end of the arm portion 13 close to the connecting member 1021, the volume of the end of the arm portion 13 close to the connecting member 1021 can be increased, and the interior space of the arm portion 13 at this site is increased accordingly. Therefore, a second fan 54 and a second air inlet opening 115 of larger sizes can be arranged on the end of the arm portion 13 close to the connecting member 1021, to thereby increase the air intake volume of the second fan 54, enhancing the air blowing and cooling effect and heat dissipation effect of the second fan 54.

The width direction of the arm portions 13 is the direction perpendicular to the length direction of the arm portion 13. In the instant embodiment, the main body 11 is of a circular ring shape, and the length direction of the arm portion 13 is the circumferential direction of the main body 11, and the width direction of the arm portions 13 may refer to the axial direction of the main body 11 or a radial direction of the main body 11. Specifically, the protrusion portion 12 protrudes outward along the axial direction of the main body 11, and the protrusion portion 12 and the second air outlet opening 113 are located on two opposite sides of the arm portions 13.

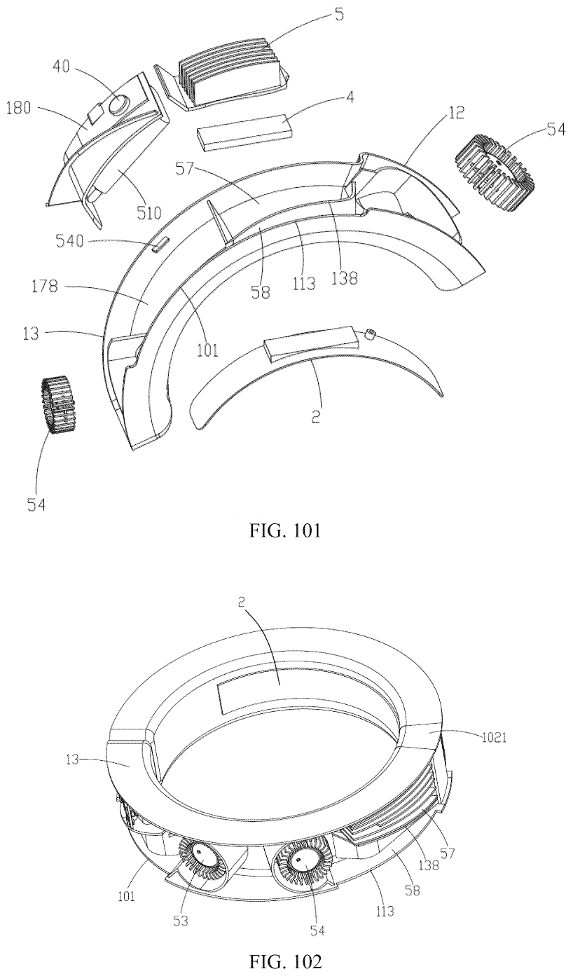

Referring to FIGS. 99 and 101, the temperature regulation assembly 50 further comprises a heat dissipating member 5. The heat dissipating member 5 is located in the first air egress path 57 and is connected to one side of the temperature regulation member 4 away from the temperature conducting member 2. When the temperature regulation member 4 operate to cool the user, the heat dissipating member 5 absorbs the heat generated by the temperature regulation member 4. The heat dissipating member 5 can increase the area for contacting with air, and can speed up heat exchange with air, and improve the heat dissipation effect of the temperature regulation member 4. Further, air in the second air egress path 58 may flow through the heat dissipating member 5 to bring away the heat of the heat dissipating member 5 during movement toward the heat dissipation opening 118, thereby improving the heat dissipation effect of the heat dissipating member 5.

In the instant embodiment, the heat dissipating member 5 comprises a base plate and a plurality of heat dissipating plates arranged at intervals on the base plate. A heat dissipation channel is formed between two adjacent ones of the heat dissipating plates. The extension direction of the heat dissipation channel is the same as the extension direction of the first air egress path 57, that the air flowing directions are the same, so as to be conducive to reducing wind resistance and speeding up air flowing.

A graphene layer (not shown) is arranged on the outside surface of the heat dissipating member 5. The graphene layer may increase the thermal conduction efficiency to help further enhance the heat dissipation effect of the heat dissipating member 5.

The temperature regulation member 4 has a cold end and a hot end, and the cold end and the hot end may be switched with each other according to the direction of current. When the temperature needs to be lowered, the cold end can be connected to the temperature conducting member 2, so that the temperature conducting member 2 may transfer the cold flow generated at the cold end to the user's neck to form a cooling effect to the user; when the temperature needs to be raised, the hot end can be connected to the temperature conducting member 2, so that the temperature conducting member 2 may transfer the hot flow generated at the hot end to the user's neck to form a warming effect to the user. Further, a heating element, such as a resistance filament, may be arranged in the chamber 178 to correspond to the first fan 53 and the second fan 54. When air is driven by the first fan 53 and the second fan 54 to flow through the heating element, the air is heated by the heating element so that the first fan 53 and the second fan 54 may blow out hot airflow to provide the user with an effect of blowing air and warming.

The temperature conducting member 2 extends from one end of the arm portion 13 close to the connecting member 1021 in a direction away from the connecting member 1021. Since the connecting member 1021 and its surrounding area are both areas close to the back of the user's neck, and can be brought into contact with the user under the action of gravity, so that making the temperature conducting member 2 extending from one end of the arm portions 13 close to the connecting member 1021 in a direction away from the connecting member 1021 may ensure that at least a portion of the temperature conducting member 2, namely the portion close to the connecting member 1021, may be in contact with the user to transmit the cold flow generated by the temperature regulation member 4 to the user. Specifically, in the instant embodiment, the second fan 54 is located between the connecting member 1021 and the temperature regulation member 4, and one end of the temperature conducting member 2 is closer to the connecting member 1021 than the second fan 54, that is in the radial direction of the main body 11, the projection cast by the second fan 54 is at least partly located on the temperature conducting member 2.

Referring to FIG. 101, each arm portion 13 is equipped with a control assembly, and the control assembly comprises a circuit board 180, a battery 510, and an ON/OFF switch 40. The battery 510, the first fan 53, the second fan 54, and the temperature regulation member 4 are all electrically connected to the circuit board 180. The battery 510 is used to supply electrical energy to the first fan 53, the second fan 54, and the temperature regulation member 4. The ON/OFF switch 40 is used to allow the user to operate and control starting and stopping of the first fan 53, the second fan 54, and the temperature regulation member 4.

The type of the battery 510 is not limited. In the instant embodiment, the battery 510 is a rechargeable battery. The arm portion 13 is provided with a charging port 540 for charging the battery 510.

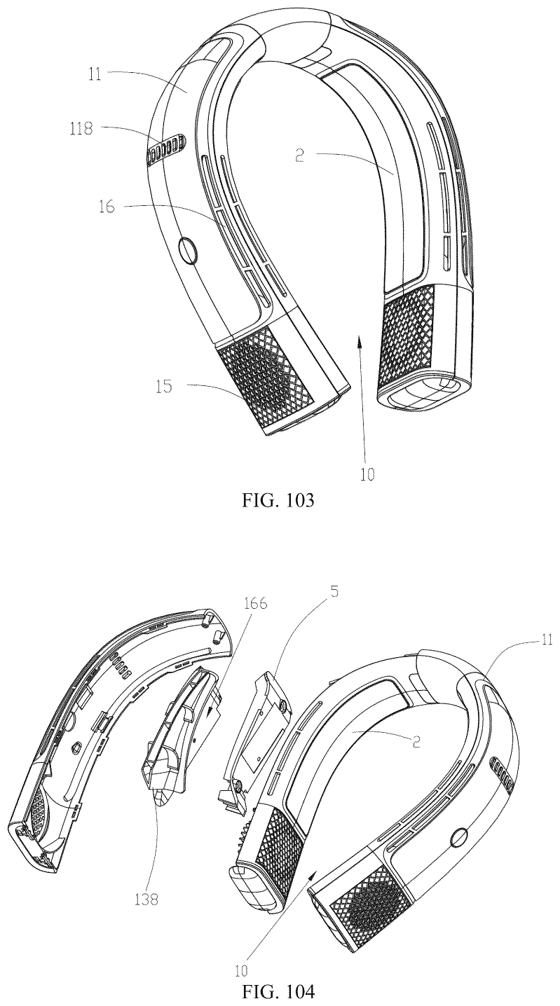

As shown in FIGS. 103-106, a twenty-first embodiment of the application provides a portable temperature regulation device, compared with the portable temperature regulation device of the first embodiment, in the instant embodiment, the portable temperature regulation device comprises a main body 11, a temperature regulation assembly 50, and a thermal isolation piece 70. The temperature regulation assembly 50 comprises a temperature regulation member 4, a heat dissipating member 5, and a temperature conducting member 2. The temperature regulation member 4 is specifically a semiconductor cooling sheet. The heat dissipating member 5 is arranged in the interior of the main body 11. The temperature conducting member 2 is fixedly connected to the main body 11, and at least a portion of the temperature conducting member 2 is exposed outside the main body 11. One side of the temperature regulation member 4 is in thermal conduction connection with the heat dissipating member 5, and the other side of the temperature regulation member 4 is in thermal conduction connection with the temperature conducting member 2. The thermal isolation piece 70 is located between the temperature conducting member 2 and the heat dissipating member 5, and the thermal isolation piece 70 is arranged to avoid the portion of the temperature regulation member 4 in thermal conduction connection with the temperature conducting member 2. The temperature regulation member 4 transmits cold to the temperature conducting member 2 when cooling, and also transmit heat to the heat dissipating member 5 at the same time, so that when the temperature conducting member 2 is in contact with the human skin, the temperature conducting member 2 may cool and lower temperature the human body, and the heat dissipating member 5 dissipates heat to the outside of the main body 11. By setting the thermal isolation piece 70 between the temperature conducting member 2 and the heat dissipating member 5 and having the thermal isolation piece 70 avoid the site of the temperature regulation member 4 in thermal conduction connection with the temperature conducting member 2, the arrangement of the thermal isolation piece 70 does not affect thermal conduction connection of the temperature regulation member 4 with the temperature conducting member 2, and can also avoid the situation that the heat dissipating from the heat dissipating member 5 irradiates the temperature conducting member 2 to cause temperature rise of the temperature conducting member 2, ensuring that the cold of the temperature conducting member 2 is not affected by the heat dissipating from the heat dissipating member 5, allowing the temperature conducting member 2 to sufficiently transmit cold to the human body to thereby improve the capability of the portable temperature regulation device for cooling and lowering temperature on the human body.

It should be noted that in the application, “thermal conduction connection” refers to direct contact between two objects to achieve transfer of heat or cold, or indirect contact to achieve transfer of heat or cold, such as indirect contact made with an intermediate heat conducting media, such as thermal silicon grease/silicone to achieve transfer of heat or cold.

It can be understood that when heating, the temperature regulation member 4 transmits heat to the temperature conducting member 2 and simultaneously transmits cold to the heat dissipating member 5, so that when the temperature conducting member 2 is in contact with the human skin, the temperature conducting member 2 provides heat for warming the human body, while the heat dissipating member 5 dissipates cold to the outside of the main body 11. At this time, energy transfer between the temperature conducting member 2 and the heat dissipating member 5 can still be isolated by the thermal isolation piece 70.

In the instant embodiment, the thermal isolation piece 70 is provided with an avoiding opening 75. The temperature regulation member 4 extends through the avoiding opening 75 to get into thermal conduction connection with the temperature conducting member 2. Since the area of the side of the temperature conducting member 2 facing the temperature regulation member 4 is larger than the area of the side of the temperature regulation member 4 facing the temperature conducting member 2, and only a portion of the surface of the side of the temperature conducting member 2 close to the heat dissipating member 5 is in thermal conduction connection with the temperature regulation member 4, to prevent another portion of the surface of the side of the temperature conducting member 2 close to the heat dissipating member 5 from being affected by the heat radiation dissipating from the heat dissipating member 5, the thermal isolation piece 70 uses the avoiding opening 75 to avoid the temperature regulation member 4. The thermal isolation piece 70 shields and covers the portion of the surface of the side of the temperature conducting member 2 close to the heat dissipating member 5 not connected to the temperature regulation member 4 in order to block the influence caused by the heat dissipating member 5 transferring heat to the temperature conducting member 2, ensuring that the temperature conducting member 2 can transfer the cold transferred from the temperature regulation member 4 to the human body as much as possible for cooling and dissipating heat for the human body. In another embodiment, the thermal isolation piece 70 is provided with an avoiding opening 75, and one side of the temperature conducting member 2 close to the heat dissipating member 5 is raised to form a raised platform 20, and the raised platform 20 extends through the avoiding opening 75 to get into thermal conduction connection with the temperature regulation member 4. The thermal isolation piece 70 is arranged on the surface of the temperature conducting member 2 close to the heat dissipating member 5 and is arranged to avoid the raised platform 20.

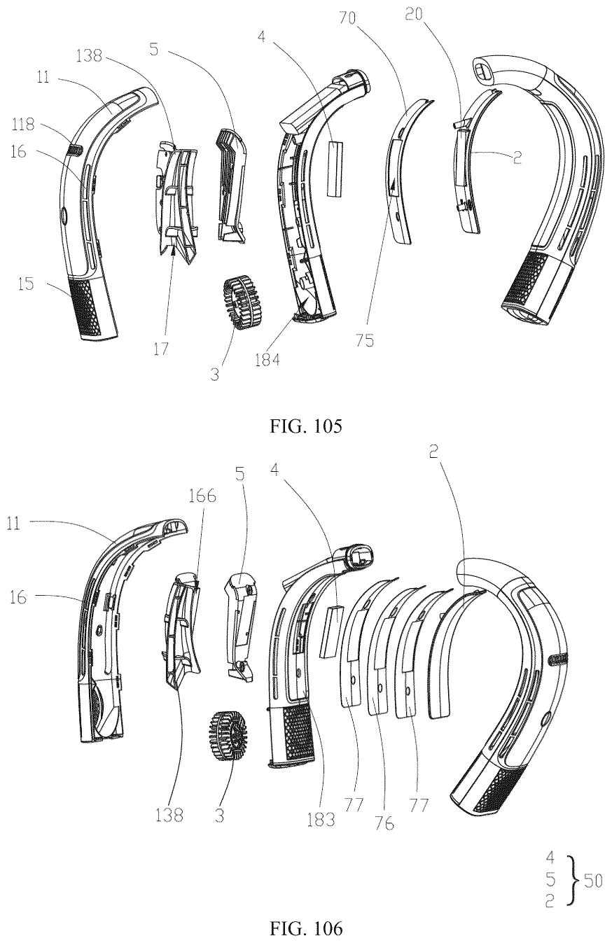

As shown in FIGS. 105 and 106, in the instant embodiment, the thermal isolation piece 70 comprises a thermal insulation layer 76 and a reflection layer 77. The reflection layer 77 is arranged on one side of the thermal insulation layer 76 close to the heat dissipating member 5. The thermal insulation layer 76 can effectively block transfer of the heat dissipating from the heat dissipating member 5 toward the temperature conducting member 2. The reflection layer 77 is located on one side of the thermal insulation layer 76 close to the heat dissipating member 5, so that when the heat dissipating from the heat dissipating member 5 radiates toward the temperature conducting member 2, the reflection layer 77 can reflect the heat dissipating from the heat dissipating member 5 to further enhance the thermal isolation capability of the thermal isolation piece 70.

Alternatively, the reflection layer 77 is arranged on one side of the thermal insulation layer 76 close to the temperature conducting member 2. The thermal insulation layer 76 prevents heat dissipating from the heat dissipating member 5 from transmitting to the temperature conducting member 2; and when the cold generated by the temperature regulation member 4 is transmitted to the temperature conducting member 2, a portion of the cold of the temperature conducting member 2 may dissipate toward the reflection layer 77, and the reflection layer 77 may reflect the portion of the cold of the temperature conducting member 2 so that the cold dissipating from the temperature conducting member 2 may concentrate and transmit toward the human skin, thereby having the temperature conducting member 2 sufficiently transmit the cold to the human body for cooling and lowering temperature for the human body.

Further, the side of the thermal insulation layer 76 close to the temperature conducting member 2 and the side of the thermal insulation layer 76 close to the heat dissipating member 5 are both provided with the reflection layer 77 in order to further enhance the thermal insulation effect of the thermal isolation piece 70 and to ensure that the temperature conducting member 2 may transfer as much cold as possible to the human skin for cooling and lowering temperature for the human body.

In the instant embodiment, the thermal insulation layer 76 is an aerosol layer. The aerosol layer is located between the temperature conducting member 2 and the heat dissipating member 5, and the aerosol layer is arranged to avoid the portion of the temperature regulation member 4 in thermal conduction connection with the temperature conducting member 2. Specifically, the aerosol layer is a nano-silicon aerosol layer. The pore size of the nano-silicon aerosol is 20-50 nm, and the nanometer-grade pore size is smaller than the mean free path of air molecules, the porosity being as high as 95%, the density being as low as 0.03 g/ml or less, achieving a vacuum-like thermal insulation effect, allowing the cold of the temperature conducting member 2 to be transmitted as much as possible to the human body.

In the instant embodiment, the reflection layer 77 uses an aluminum foil reflection layer. The aluminum foil reflection layer is located between the temperature conducting member 2 and the heat dissipating member 5, and the aluminum foil reflection layer avoids the portion of the temperature regulation member 4 in thermal conduction connection with the temperature conducting member 2. Specifically, the aluminum foil reflection layer is formed through laminating an aluminum foil surface, a polyethylene film, a fiber braid, and a metal coating film with hot melt adhesive. The aluminum foil reflection layer reflects the heat or cold irradiating the aluminum foil reflection layer, that is when the heat dissipating member 5 radiates heat toward the aluminum foil reflection layer, the aluminum foil reflection layer reflects the heat back so that the heat of the heat dissipating member 5 does not irradiate on the temperature conducting member 2; and when the temperature conducting member 2 radiates cold toward the aluminum foil reflection layer, the aluminum foil reflection layer reflects the cold back so that the cold of the temperature conducting member 2 can be concentrated and radiating toward the human body to transfer the cold as much as possible to the human body.

In the instant embodiment, the temperature conducting member 2 is a metal temperature conducting member mixed with graphene power. By adding graphene powder, the thermal conduction efficiency of the temperature conducting member 2 can be further enhanced.

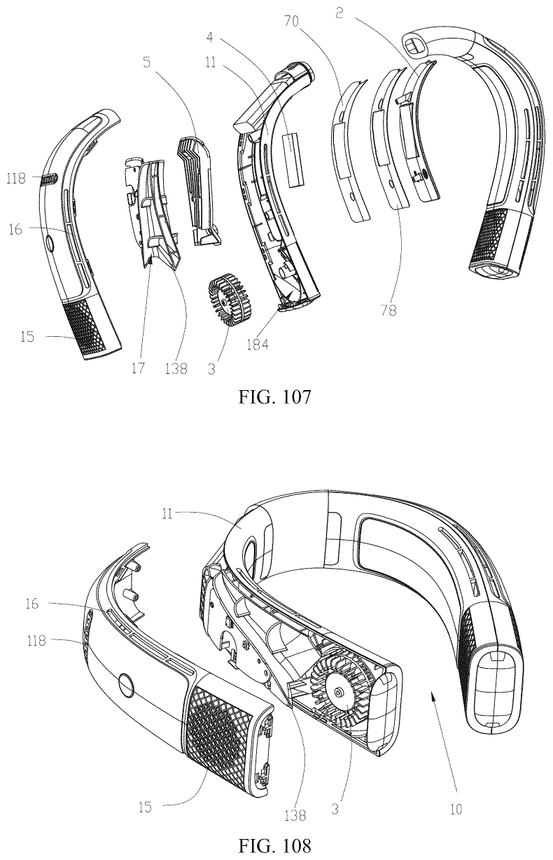

As shown in FIGS. 107 and 108, in a twenty-second embodiment, compared with the twenty-first embodiment, in the instant embodiment, the portable temperature regulation device comprises a temperature uniform component 78. The temperature uniform component 78 is located between the thermal isolation piece 70 and the temperature conducting member 2. The thermal isolation piece 70 may comprise at least one of the thermal insulation layer 76 and the reflection layer 77. The temperature uniform component 78 is specifically a graphene layer or a graphite layer. The temperature uniform component 78 covers one side of the temperature conducting member 2 close to the temperature regulation member 4. When the temperature regulation member 4 transmits cold to the temperature conducting member 2, the temperature uniform component 78 can quickly spread the cold of the portion of the temperature conducting member 2 close to the temperature regulation member 4 on the temperature uniform component 78 and transmit to the temperature conducting member 2, so as to make the cold at various portions of the temperature conducting member 2 more uniform. When the temperature conducting member 2 is in contact with the human body, the temperature conducting member 2 uniformly transfer the cold to the human body. The temperature uniform component 78 is connected to the temperature conducting member 2 by adhesive, and the adhesive is a heat-conducting adhesive.

As shown in FIGS. 103-108, in the twenty-first embodiment and the twenty-second embodiment, the temperature conducting member 2 is arranged on an outside surface of the main body 11, and the thermal isolation piece 70 is located between the outside surface of the main body 11 and the temperature conducting member 2. The temperature conducting member 2 is fixedly connected to the main body 11, and the temperature conducting member 2 clamps and fixes the thermal isolation piece 70 on the main body 11. In other embodiments, the thermal isolation piece 70 may be fixed to the main body 11 or the temperature conducting member 2 by means of gluing or snapping. When the thermal isolation piece 70 comprises a thermal insulation layer 76 and a reflection layer 77, the thermal insulation layer 76 is connected to the reflection layer 77 by adhesive, or the thermal insulation layer 76 and the reflection layer 77 are stacked and are clamped and fixed to the main body 11 by the temperature conducting member 2. Since the thermal isolation piece 70 is located between the main body 11 and the temperature conducting member 2, the thermal isolation piece 70 prevents the heat emitting from the heat dissipating member 5 from being radiated to the temperature conducting member 2 through the main body 11, and the thermal isolation piece 70 prevents the temperature conducting member 2 from transferring a portion of cold to the main body 11, ensuring the cold of the temperature conducting member 2 is not consumed by the main body 11 to thereby ensure the cold is sufficient cold, allowing the temperature conducting member 2 to transfer cold as much as possible to the human body, thereby solving the problem that the cold transferred from the temperature conducting member 2 to the human body is greatly reduced and also enhancing the capability of the portable temperature regulation device to cool and dissipate heat for the human body.

In other embodiments, the temperature conducting member 2 may also be arranged on an inside surface of the main body 11, that is the temperature conducting member 2 is located inside the main body 11, and at least a part of the temperature conducting member 2 protrudes outward outside of the main body 11 and exposed outside of the main body 11. The thermal isolation piece 70 may be fixed on one side of the temperature conducting member 2 close to the heat dissipating member 5 by adhesive, and does not need to be clamped and fixed on the main body 11 by the temperature conducting member 2.

Further, the portable temperature regulation device can be a neck-mounted air conditioner, a handheld air conditioner, a head-mounted air conditioner, or a neck-mounted air conditioner. In the following, a description will be made by taking the portable temperature regulation device as a neck-mounted air condition. Specifically, the main body 11 encloses and forms a wear space 10, and the temperature conducting member 2 is located on one side of the main body 11 close to the wear space 10. When a user wears the portable temperature regulation device on the neck, the neck is located in the wear space 10, and the skin of the neck is in contact with the temperature conducting member 2. Namely, when the temperature regulation member 4 is in operation, the cold generated by the temperature regulation member 4 is transferred to the temperature conducting member 2, and the temperature conducting member 2 then transmits the cold to the skin of the neck.

Further, one side of the main body 11 close to the wearing cavity 21 is recessed to form a receiving trough 183, and the thermal isolation piece 70 and the temperature conducting member 2 are located in the receiving trough 183 to thereby reduce the overall thickness of the portable temperature regulation device. Specifically, the temperature conducting member 2 is fixedly connected to the main body 11, and the thermal isolation piece 70 is clamped between the trough bottom of the receiving trough 183 and the temperature conducting member 2.

As shown in FIGS. 103-108, specifically, the main body 11 is provided, in the interior thereof, with a mounting chamber 184 and a disposition compartment in communication with each other. The portable temperature regulation device further comprises a partition member 138 and a fan 3. The fan 3 is arranged in the mounting chamber 184, and the partition member 138 is arranged in the disposition compartment and divides the disposition compartment into a heat dissipation chamber 166 and an air channel 17. The heat dissipation chamber 166 and the air channel 17 are both in communication with the mounting chamber 184. The heat dissipating member 5 is arranged in the heat dissipation chamber 166. One side of the main body 11 adjacent to the wear space 10 is provided with an air outlet opening 16, and the air outlet opening 16 is in communication with one end of the air channel 17 away from the mounting chamber 184. One side of the main body 11 opposite to the wear space 10 is provided with a heat dissipation opening 118, and the heat dissipation opening 118 is in communication with one end of the heat dissipation chamber 166 away from the mounting chamber 184. The main body 11 is also provided with an air inlet opening 15 in communication with the mounting chamber 184. Namely, when the fan 3 operates inside the mounting chamber 184, air drawn in from the air inlet opening 15 is sped up in the mounting chamber 184 to then form airflows respectively blown toward the heat dissipation chamber 166 and the air channel 17, and the airflow passing through the heat dissipation chamber 166 bring away heat emitting from the heat dissipating member 5 to blow out from the heat dissipation opening 118, and the airflow passing through the air channel 17 blows out from the air outlet opening 16. When the user wears the portable temperature regulation device on the neck, airflow blown out of the air outlet opening 16 blows toward the face and the likes to cool the human body, and the temperature conducting member 2 transmits the cold to the skin of the neck to further enhance the capability of cooling and lowering temperature for the human body.

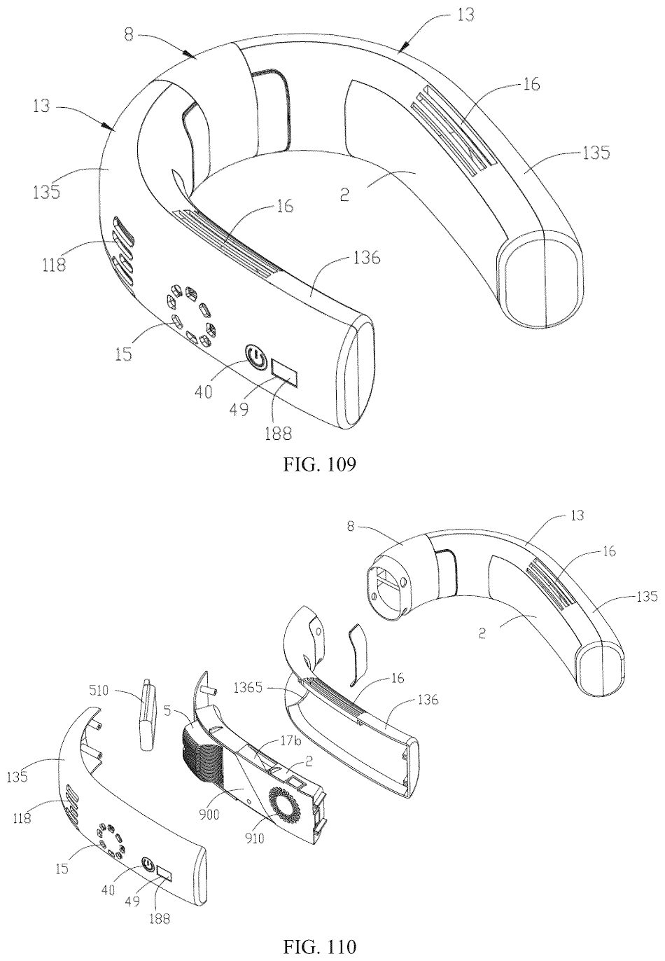

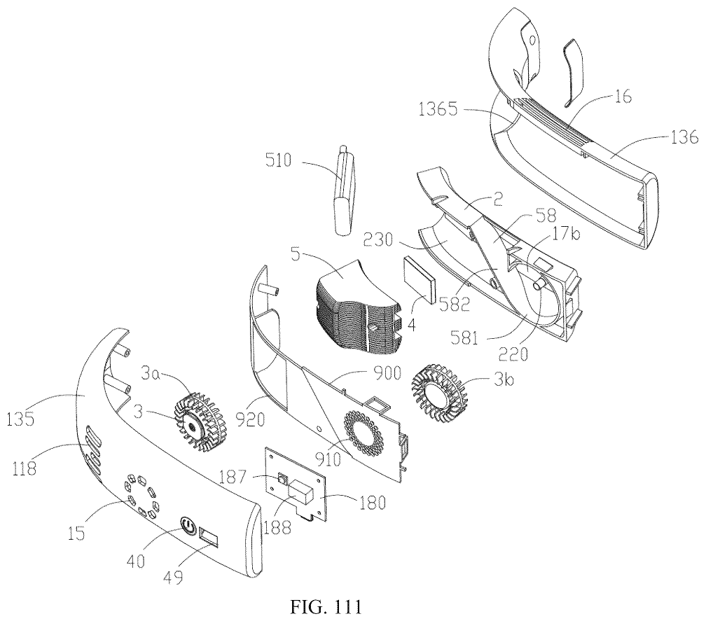

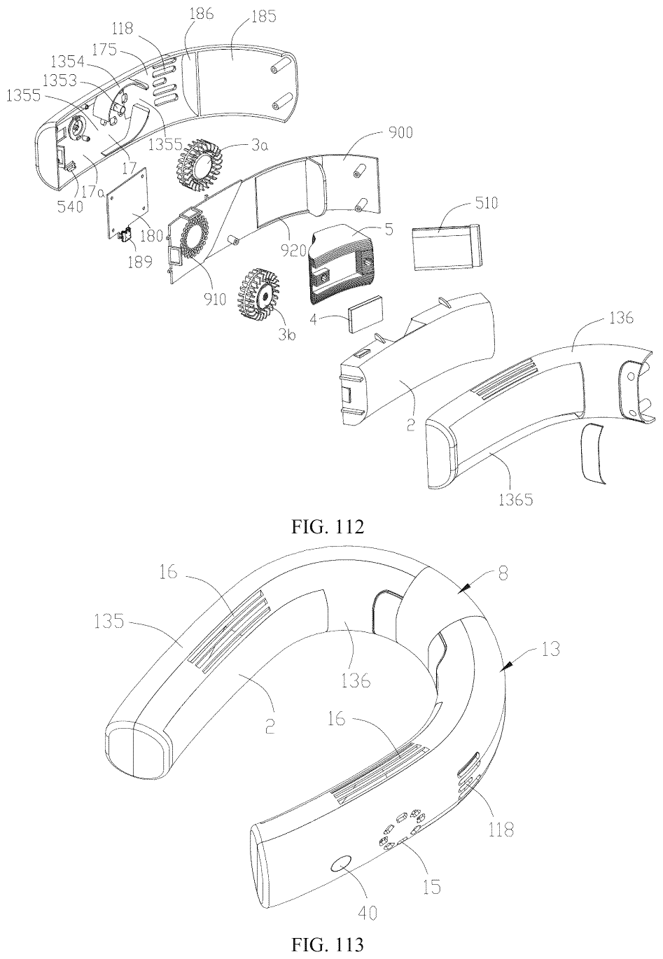

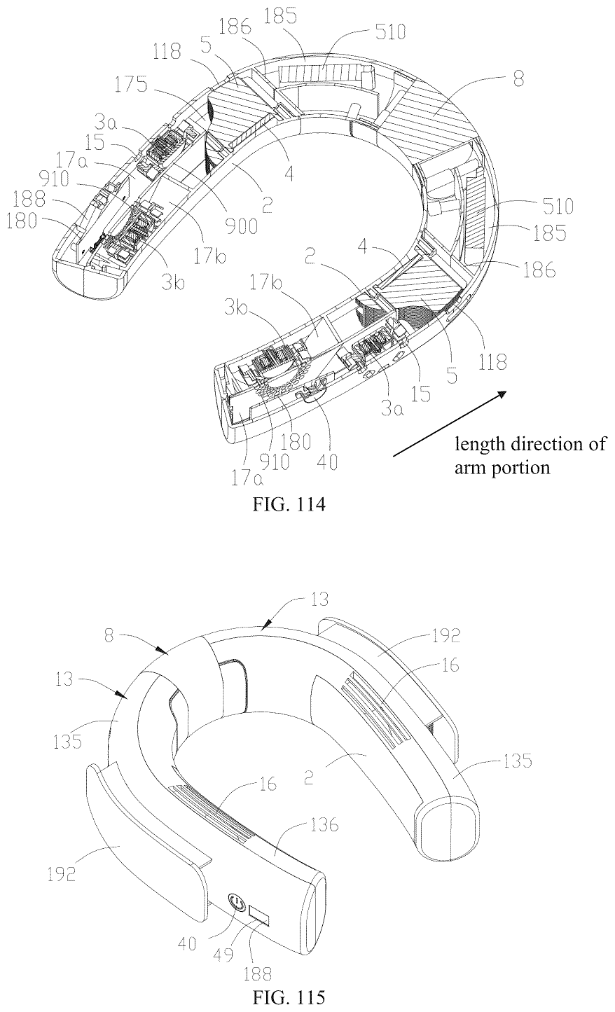

Referring jointly to FIGS. 109-114, a twenty-third embodiment of the application provides a portable temperature regulation device with good air outlet temperature regulation effect, compared with the first embodiment, in the instant embodiment, the portable temperature regulation device comprises arm portions 13 each being provided with an air channel 17. A conducting member 900 is arranged in the air channel 17 to divide the air channel 17 at least into a first air channel 17a and a second air channel 17b, the first air channel 17a and the second air channel 17b communicating with each other. Each arm portion 13 is provided with an air inlet opening 15 at a location corresponding to the first air channel 17a, and an air outlet opening 16 at a location corresponding to the second air channel 17b. A fan 3 is arranged in the air channel 17. A temperature regulation member 4 is arranged in the arm portion 13 and in thermal conduction connection with the conducting member 900.

It should be noted that in the application, “thermal conduction connection” refers to direct contact between two objects to achieve transfer of heat, or indirect contact to achieve transfer of heat, such as indirect contact made with an intermediate heat conducting media, such as thermal silicon grease/silicone to achieve transfer of heat.

The portable temperature regulation device of this embodiment provides cooling or heating to the conducting member 900 through the temperature regulation member 4 and uses the conducting member 900 to divide the air channel 17 into at least the first air channel 17a and the second air channel 17b, so that the airflow flowing in from the air inlet opening 15 is brought into contact with the conducting member 900 when passing through the first air channel 17a and the second air channel 17b to be fully cooled or heated, to finally have the airflow blown out of the air outlet opening 16 become cooler or warmer, thereby providing the portable temperature regulation device with a better temperature regulation effect.

The temperature regulation member 4 is preferably a semiconductor cooling sheet, and when supplied with electrical power, two opposite ends of the temperature regulation member 4 respectively form a cold end and a hot end. The cold end and the hot end may be switched with each other by changing the direction of current supplied to the temperature regulation member 4. When cooling is desired, the cold end of the temperature regulation member 4 is set in thermal conduction connection with a component to be temperature-adjusted (such as the conducting member 900), so as to get cooled when airflow flows through the first air channel 17a and the second air channel 17b to make the airflow blown out of the air outlet opening 16 cooler. Oppositely, when heating is desired, the hot end of the temperature regulation member 4 is set in thermal conduction connection with a component to be temperature-adjusted (such as the conducting member 900), so as to get heated when airflow flows through the first air channel 17a and the second air channel 17b to make the airflow blown out of the air outlet opening 16 warmer.

Optionally, the arm portion 13 comprises an inside casing 136 and an outside casing 135 buckled and mounted together. The air channel 17 is formed between the inside casing 136 and the outside casing 135. Specifically, the first air channel 17a is formed between the outside casing 135 and the conducting member 900, and the first air channel 17a extends along the length direction of the arm portion 13. The air inlet opening 15 is provided on a side wall of the outside casing 135. The second air channel 17b is formed between the inside casing 136 and the conducting member 900, and the second air channel 17b extends along the length direction of the arm portion 13 and is located on the inner side of the first air channel 17a. The air outlet opening 16 is provided on a top wall of the inside casing 136. Optionally, the air outlet opening 16 comprises a plurality of air outlet holes in the form of strips arranged side by side.

Optionally, the air channel 17 further comprises an air channel 175 in communication with the first air channel 17a. The arm portion 13 is provided with a heat dissipation opening 118 at a location corresponding to the air channel 175. Specifically, the heat dissipation opening 118 is provided on the side wall of the outside casing 135. The heat dissipation opening 118 comprises a plurality of heat dissipation holes in the form of strips arranged side by side.

Optionally, the outside casing 135 is provided, in the interior thereof, with a first rotary axle 1353 extending toward the first air channel 17a and an air squeezing member 1354 arranged around the periphery of the first rotary axle 1353. Specifically, the air squeezing member 1354 is arranged in one end of the first air channel 17a, and two opposite ends of the air squeezing member 1354 are each formed with an opening 1355, wherein one of the openings 1355 faces the other end of the first air channel 17a and the other one of the openings 1355 faces the air channel 175.

Optionally, the air inlet opening 15 comprises a plurality of air inlet apertures arranged in a ring shape. The plurality of air inlet apertures is spaced and arranged around the periphery of the first rotary axle 1353 and is located between the first rotary axle 1353 and the air squeezing member 1354.

Optionally, the fan 3 comprises a first fan 3a located in the first air channel 17a and arranged at a location corresponding to the air inlet opening 15. Specifically, the first fan 3a is rotatably mounted on the first rotary axle 1353. Preferably, the fan 3 is a centrifugal fan, and the first fan 3a is arranged in one end of the first air channel 17a close to the air channel 175, and air inlet side of the first fan 3a is arranged to face the air inlet opening 15, and the air outlet side of the first fan 3a faces the other end of the first air channel 17a away from the air channel 175 and the air channel 175. The first fan 3a guides a part of the airflow into the other end of the first air channel 17a away from the air channel 175, and the first fan 3a guides another part of the airflow into the air channel 175, so that to dissipate the heat generated by the temperature regulation member 4 to the outside from the heat dissipation opening 118.

Optionally, the outside casing 135 is further provided with a battery compartment 185 formed in the interior thereof. The battery compartment 185 is formed between the outside casing 135 and the conducting member 900. The battery compartment 185 and the air channel 175 are separated from each other by a separation plate 186. A battery 510 is disposed in the battery compartment 185, and the battery 510 is used to supply electrical power to the fan 3.

Optionally, the conducting member 900 is made of a heat conductive material, such as a metal material. In the instant embodiment, the conducting member 900 is made of an aluminum material to better transfer cold or heat generated by the temperature regulation member 4 to the first air channel 17a and the second air channel 17b for improving temperature regulation efficiency.

Optionally, the conducting member 900 is provided with an air passage opening 910, and the first air channel 17a and the second air channel 17b communicate with each other through the air passage opening 910. Preferably, the air passage opening 910 comprises a plurality of air passage apertures arranged in a ring shape. The second fan 3b is arranged to correspond, in position, to the air passage opening 910. The air inlet side of the second fan 3b is arranged to face the air passage opening 910, so that airflow is driven by the second fan 3b to flow from the first air channel 17a through the air passage opening 910 into the second air channel 17b.

In another embodiment, there may be no air passage opening 910 on the conducting member 900, and under such a circumstance, the air channel 17 may further comprise an air passage channel, and the air passage channel is located at one end of the conducting member 900. The first air channel 17a and the second air channel 17b are located at two opposite ends of the conducting member 900, and the first air channel 17a and the second air channel 17b are set in communication with each other through the air passage channel. The fan 3 is arranged in the air passage channel.

Optionally, the conducting member 900 may be further provided with a through hole 920, and the through hole 920 is arranged to face the heat dissipation opening 118.

Optionally, the first air channel 17a is arranged to extend along the length direction of the arm portion 13. The air inlet opening 15 and the air passage opening 910 are respectively at two ends of the first air channel 17a, so that the air inlet opening 15 and the air passage opening 910 are arrange to shift away from each other in the inside-outside direction of the arm portion 13 so as to increase the length of the first air channel 17a, allowing the airflow to be fully cooled or heated in the first air channel 17a. Of course, in other embodiments, the air inlet opening 15 and the air passage opening 910 can also be arranged at the same end of the first air channel 17a, and under this condition, the air inlet opening 15 is arranged to face the air passage opening 910, and the first fan 3a is located between the air inlet opening 15 and the air passage opening 910.

Optionally, a circuit board 180 is further disposed in the first air channel 17a, and the circuit board 180 is arranged to face the air passage opening 910. The circuit board 180 is electrically connected with the battery 510, the fan 3, and the temperature regulation member 4. Specifically, the circuit board 180 is provided with an ON/OFF button 187, a display screen 188, and a charging terminal 189. The side wall of the outside casing 135 is provided with a pushbutton 40 for cooperating with the ON/OFF button 187 and a display window 49 for displaying the display screen 188. The pushbutton 40 is used to control starting and stopping and speed position of the fan 3 and the temperature regulation member 4. The display screen 188 is used to display the power capacity information of the battery 510, the speed position information of the fan 3, the temperature adjustment information of the temperature regulation member 4. The bottom wall of the outside casing 135 is provided with a charging port 540 into which the charging terminal 189 extends. The charging terminal 189 is used to connect with an external power supply for charging the battery 510.

Optionally, the component to be temperature-adjusted may further include a temperature conducting member 2. In the instant embodiment, the side wall of the inside casing 136 is provided with a first installation opening 1365 and a temperature conducting member 2 mounted in the first installation opening 1365. The temperature conducting member 2 is exposed on the side wall of the inside casing 136 and is set in thermal conduction connection with the conducting member 900. The temperature conducting member 2 is used to contact the user's part to be temperature-adjusted, so as to perform ice or heat compress temperature control.

Optionally, the temperature conducting member 2 and the conducting member 900 are mounted through buckling, and the second air channel 17b is formed between the temperature conducting member 2 and the conducting member 900, so that when the airflow flows through the second air channel 17b, the temperature conducting member 2 and the conducting member 900 can both function to adjust temperature for the airflow so as to enhance the temperature regulation function for the airflow.

Optionally, the temperature conducting member 2 is made of a heat conductive material, such as a metal material. In the instant embodiment, the temperature conducting member 2 is made of an aluminum material to better transfer the cold or heat generated by the temperature regulation member 4 to the user's part to be temperature-adjusted, improving the efficiency of temperature regulation.

Optionally, a second rotary axle 220 is formed on the inner side of the temperature conducting member 2 and extends toward the second air channel 17b. The fan 3 comprises a second fan 3b, and the second fan 3b is disposed in the second air channel 17b. Specifically, the second fan 3b is rotatably mounted on the second rotary axle 220. Preferably, the second fan 3b is a centrifugal fan.

In another embodiment, there may be no temperature conducting member 2 provided on the side wall of the inside casing 136. Correspondingly, the side wall of the inside casing 136 may be provided with an air inlet opening 15, and the air inlet opening 15 comprises a plurality of air inlet apertures arranged in a ring shape. The plurality of air inlet apertures is arranged around the periphery of the second rotary axle 220 to thereby increase the air intake volume of the second fan 3b and increase the wind power so as to have the airflow blown out from the air outlet opening 16 stronger.

Preferably, the fan 3 comprises a first fan 3a and a second fan 3b. The first fan 3a blows air entering from the air inlet opening 15 from one end of the first air channel 17a toward the other end of the first air channel 17a, and then the second fan 3b draws in airflow from the air passage opening 910 to pass through the second air channel 17b to the blow out from the air outlet opening 16, so that even the length of the first air channel 17a and the second air channel 17b is increased, the airflow can still smoothly enter from the air inlet opening 15 to finally blow out from the air outlet opening 16, and the airflow can be fully cooled or heated in the first air channel 17a and the second air channel 17b having an extended length to ensure the airflow blown out from the air outlet opening 16 is cooler or warmer. Of course, in another embodiment, the fan 3 may comprise only the first fan 3a or the second fan 3b.

Optionally, the component to be temperature-adjusted may further include an air guide member 58, and the air guide member 58 is connected to one side of the temperature conducting member 2. In the instant embodiment, the air guide member 58 is arranged in the arm portion 13 and connected to an inner side of the temperature conducting member 2. The air guide member 58 is in thermal conduction connection with the temperature regulation member 4. Preferably, one side of the air guide member 58 is integrally connected to the temperature conducting member 2, and the other side of the air guide member 58 is in contact with one side of the conducting member 900. Of course, the air guide member 58 and the temperature conducting member 2 can be separate structures that are mounted to fixedly connected to each other.

Optionally, the conducting member 900, the temperature conducting member 2, and the air guide member 58 jointly enclose and form the second air channel 17b. When the airflow flows through the second air channel 17b, the conducting member 900, the temperature conducting member 2, and the air guide member 58 can all perform temperature regulation for the airflow, thereby maximizing the temperature regulation effect for the airflow.

Optionally, the air guide member 58 comprises a curved air guide plate 581 and an air guide plate 582 connected to each other. The curved air guide plate 581 is arranged around the periphery of the second fan 3b. The air guide plate 582 is arranged to extend in a direction from one end of the curved air guide plate 581 toward the air outlet opening 16. The curved air guide plate 581 is used to squeeze the wind power generated by the second fan 3b, so as to make the airflow more quickly flow toward the air guide plate 582 and prevent the airflow from flowing backward. The air guide plate 582 is used to guide the airflow to the air outlet opening 16 for blowing out. Preferably, the curved air guide plate 581 and the air guide plate 582 are integrally connected.

Optionally, a mounting chamber 230 is also formed inside the temperature conducting member 2, and the mounting chamber 230 and the second air channel 17b are separated by the air guide plate 582. The temperature regulation member 4 is received in the mounting chamber 230. The temperature regulation member 4 and the temperature conducting member 2 are set in indirect contact with each other by means of thermal silicon grease/silicone to form heat transfer, so that the cold or heat generated by the temperature regulation member 4 can not only transmit to the temperature conducting member 2, but also indirectly transmit through the temperature conducting member 2 to the conducting member 900 and the air guide member 58.

In another embodiment, optionally, it is also feasible that the temperature regulation member 4 and the conducting member 900 are set in indirect contact with each other by means of thermal silicon grease/silicone to form heat transfer, or alternatively, the temperature regulation member 4 and the air guide member 58 are set in indirect contact with each other by means of thermal silicon grease/silicone to form heat transfer, so as to similarly achieve transmitting the cold or heat generated by the temperature regulation member 4 to the temperature conducting member 2, the conducting member 900, and the air guide member 58, namely realizing the temperature conducting member 2, the conducting member 900, and the air guide member 58 area separately in thermal conduction connection with the temperature regulation member 4.

Optionally, the arm portion 13 is further provided with a heat dissipating member 5 therein, and the temperature regulation member 4 is clamped between the heat dissipating member 5 and the temperature conducting member 2. A part of the heat dissipating member 5 is received in the mounting chamber 230 and is in thermal conduction connection with the temperature regulation member 4. Another part of the heat dissipating member 5 extends through the through hole 920 to get into the air channel 175, in order to quickly spread the heat generated by the temperature regulation member 4 to the air channel 175. Preferably, the heat dissipating member 5 and the temperature regulation member 4 are set in indirect contact with each other by means of thermal silicon grease/silicone to form heat transfer.

Optionally, the portable temperature regulation device is a neck-mounted air conditioner, a waist-mounted air conditioner, or a handheld air condition. In the instant embodiment, the portable temperature regulation device is a neck-mounted air conditioner. The portable temperature regulation device comprises the two arm portions 13 and an elastic restoration member 8 connecting the two arm portions 13. The two arm portions 13 are movable in directions away from each other under the action of external forces, so as to cause deformation of the elastic restoration member 8, allowing a user to easily wear the portable temperature regulation device on the neck of the human body, and after the portable temperature regulation device is worn on the neck of the human body, and the external forces acting on the two arm portions 13 are released, the automatic elastic restoration of the elastic restoration member 8 makes the temperature conducting members 2 on the inside walls of the two arm portions 13 both contact with the neck of the human body, so as to perform ice or heat compress temperature regulation for the neck of the human body. When the portable temperature regulation device is worn on the user's neck, the air outlet openings 16 of the two arm portions 13 discharge airflow toward the head of the human body or discharge airflow toward the back of the human body.

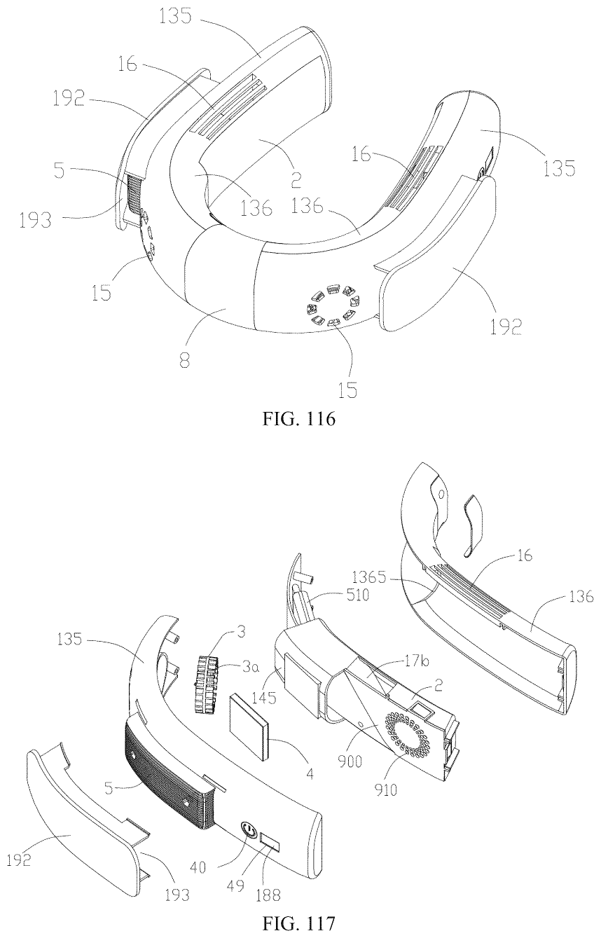

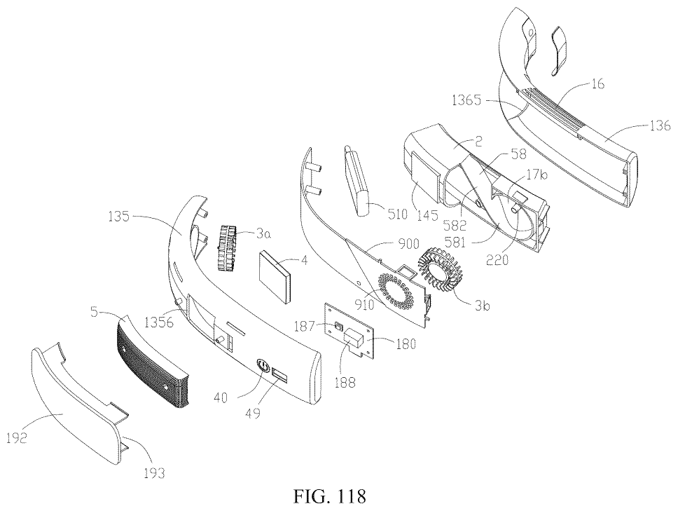

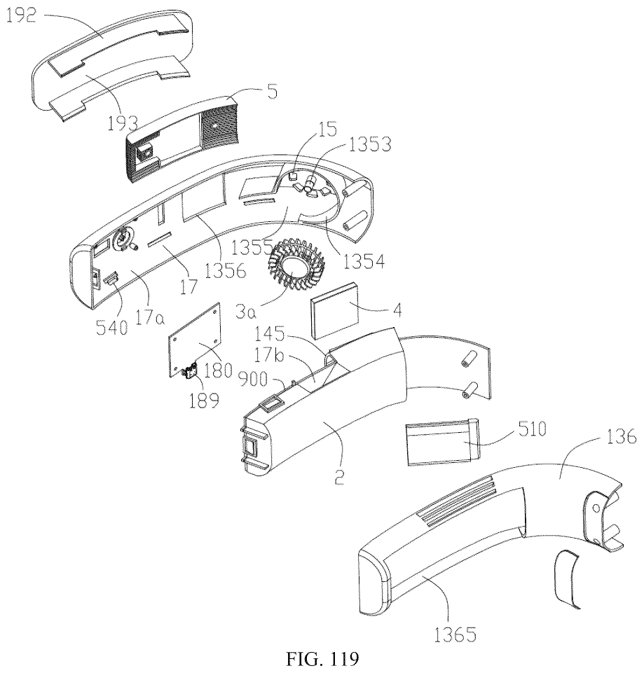

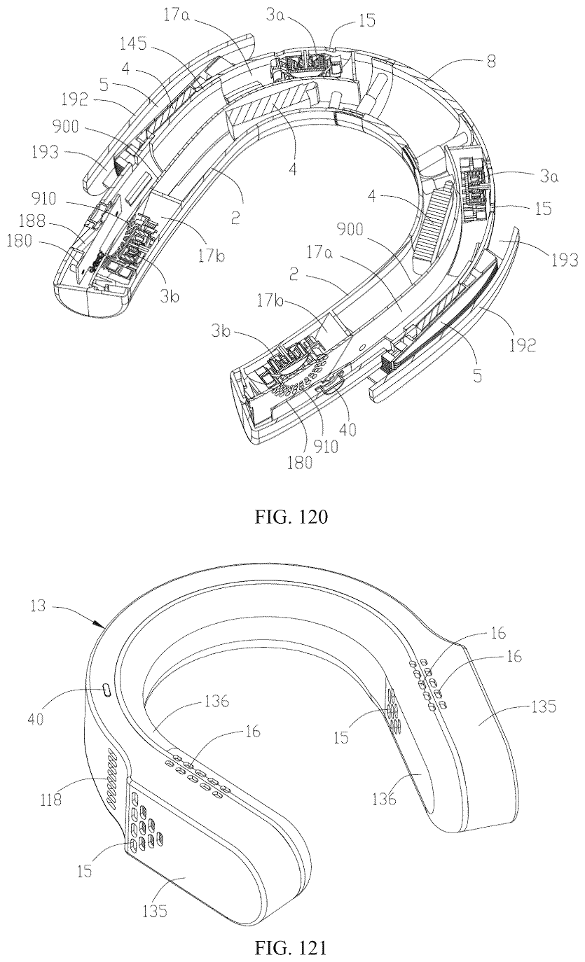

Referring jointly to FIGS. 115-120, a twenty-fourth embodiment of the application provides a portable temperature regulation device, of which the structure that is similar to that of the twenty-third embodiment described above may refer to the related description of the above-described twenty-third embodiment and the description will not be repeated for the instant embodiment. Compared with the twenty-third embodiment, in the instant embodiment, the component to be temperature-adjusted may further include a connecting member 145. The connecting member 145 is located in the first air channel 17a and abuts against the inner side of the outside casing 135. Two opposite ends of the connecting member 145 are respectively integrated with the top wall and the bottom wall of the conducting member 900. One side of the connecting member 145 away from the temperature conducting member 2 is in thermal conduction connection with one side of the temperature regulation member 4. Preferably, the connecting member 145 and the temperature regulation member 4 are set in indirect contact with each other by means of thermal silicon grease/silicone in order to form heat transfer. Correspondingly, the side wall of the outside casing 135 is provided with a second installation opening 1356 through which the temperature regulation member 4 extends. The heat dissipating member 5 is fixedly mounted to the side wall of the outside casing 135 and is in thermal conduction connection with the other side of the temperature regulation member 4. In other words, one side of the heat dissipating member 5 is in thermal conduction connection with the temperature regulation member 4, and the other side of the heat dissipating member 5 is exposed outside the side wall of the outside casing 135. Preferably, the heat dissipating member 5 and the temperature regulation member 4 are set in indirect contact with each other by means of thermal silicon grease/silicone in order to form heat transfer. The arm portions 13 further comprises a cover plate 192, and the cover plate 192 covers the heat dissipating member 5 and is fixedly mounted to the side wall of the outside casing 135. One side of the cover plate 192 facing the outside casing 135 is formed with a heat-dissipating channel 193 extending along the length direction of the outside casing 135. The heat dissipating member 5 is received in the heat-dissipating channel 193, and the heat dissipating member 5 quickly spreads the heat generated by the temperature regulation member 4 into the heat-dissipating channel 193 to be dissipated to the outside from two ends of the heat-dissipating channel 193. In other embodiments, the side wall of the arm portions 13 is provided with an opening, and the heat dissipating member 5 is arranged in the opening and is exposed to the outside of the arm portion 13, or the heat dissipating member 5 is disposed in the arm portion 13 and is exposed through the opening to the outside of the arm portion 13, and correspondingly, the arm portion 13 may not be provided with the cover plate 192, and the heat generated by the temperature regulation member 4 can similarly be quickly spread through the heat dissipating member 5 to outside of the arm portion 13.

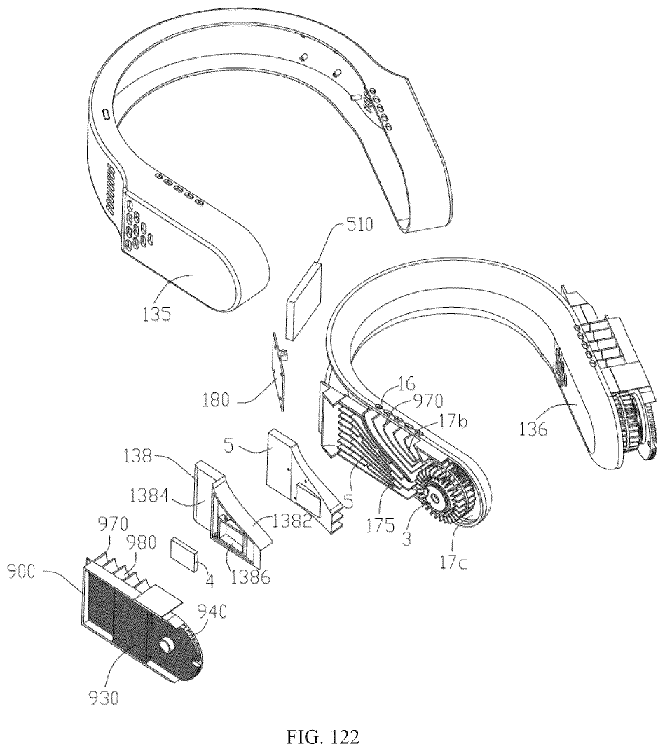

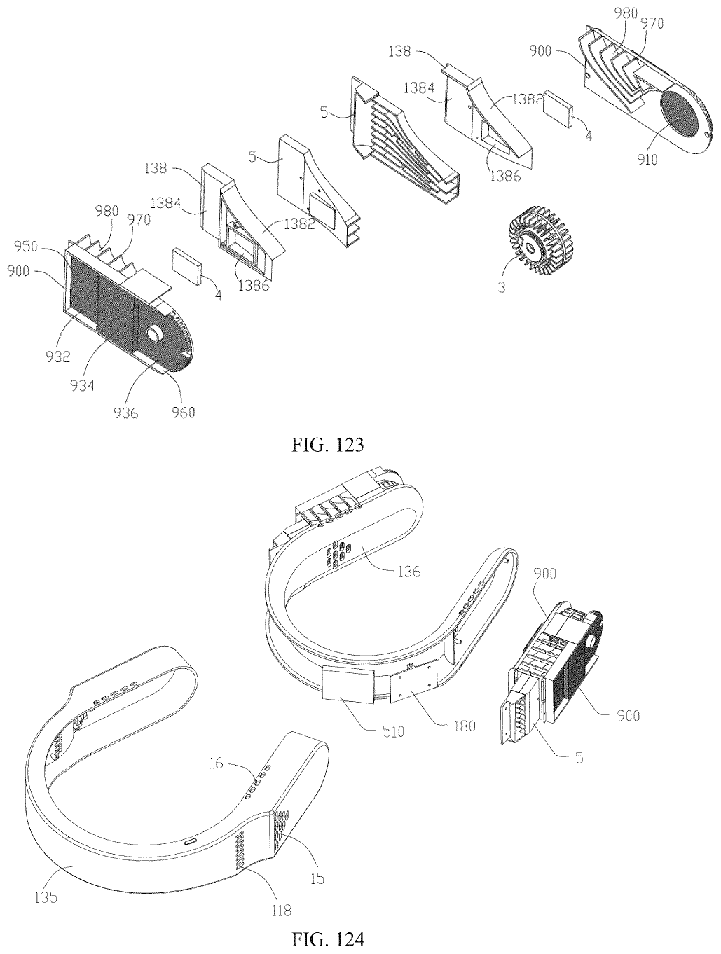

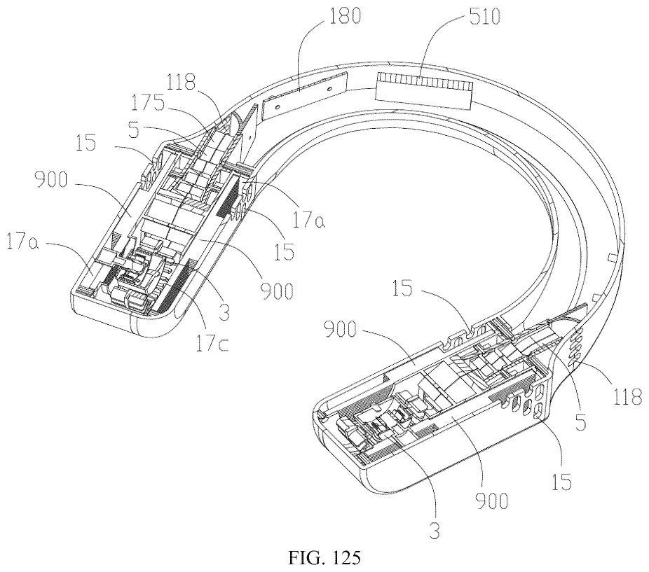

Referring to FIGS. 121-125, a twenty-fifth embodiment of the application provides a portable temperature regulation device, and compared with the twenty-third embodiment described above:

In the instant embodiment, the air channel 17 further comprises an air passage channel 17c and a heat dissipation air channel 175. The fan 3 is arranged in the air passage channel 17c which is in communication with a first air channel 17a, a second air channel 17b, and the heat dissipation air channel 175. The second air channel 17b and the air channel 175 are connected to the same end of the air passage channel 17c. The arm portion 13 comprises a heat dissipation opening 118 formed at a location corresponding to the air channel 175. A heat dissipating member 5 is arranged in the air channel 175, and the heat dissipating member 5 can quickly spread the heat generated by the temperature regulation member 4 into the air channel 175.

Optionally, the arm portion 13 is provided with a partition member 138 which comprises a first separation part 1382 and a second separation part 1384 connected to one side of the first separation part 1382. Specifically, the second separation part 1384 is integrally connected to the outer side of the first separation part 1382.

Optionally, the first separation part 1382 is located between the second air channel 17b and the air channel 175 and separates the second air channel 17b and the air channel 175, so that airflows respectively entering the second air channel 17b and the air channel 175 do not interfere with each other. The second separation part 1384 is located between the conducting member 900 and the air channel 175 and separates the conducting member 900 and the air channel 175 to prevent the airflow entering the air channel 175 from affecting the temperature conducting effect of the conducting member 900. Specifically, the second air channel 17b and the air channel 175 are respectively located at upper and lower sides of the first separation part 1382, and the air channel 175 and the conducting member 900 are respectively located at inner and outer sides of the second separation part 1384.

Optionally, the second separation part 1384 is provided with an installation opening 1386. The installation opening 1386 penetrates through inner and outer sides of the second separation part 1384. The temperature regulation member 4 is mounted in the installation opening 1386. Inner and outer sides of the temperature regulation member 4 are respectively in thermal conduction connection with the conducting member 900 and the heat dissipating member 5.

Optionally, the conducting member 900 is extended toward the interior of the first air channel 17a to form a plurality of first temperature conducting portions 930, and two adjacent first temperature conducting portions 930 form therebetween a first air guide channel 940 to further increase the contact area between the airflow and the conducting member 900, allowing the airflow to be more fully cooled or heated. Specifically, the first temperature conducting portions 930 are in a plate form and extend along the length direction of the arm portion 13. The plurality of first temperature conducting portions 930 are arranged parallel to each other. Preferably, each first temperature conducting portion 930 comprises a first portion 932, a second portion 934, and a third portion 936. The second portion 934 is connected between the first portion 932 and the third portion 936. In the inside-outside direction of the arm portion 13, the size of the second portion 934 is greater than the size of the first portion 932 and the size of the third portion 936. The second portion 934 is connected to the outside wall of the arm portion 13. The first portion 932 is arranged to correspond in position to the air inlet opening 15. The first portion 932 and the outside wall of the arm portion 13 form therebetween a first connection channel 950. The third portion 936 is arranged to correspond in position to an air passage opening 9101. The third portion 936 and the outside wall of the arm portion 13 form therebetween a second connection channel 280. This arrangement not only allows the airflow to smoothly move from the air inlet opening 15 through the first connection channel 950 to enter each first air guide channel 940, but also allows the airflow in the first air guide channel 940 having no air passage opening 910 arranged there to pass through the second connection channel 960 to smoothly flow into the first air guide channel 940 having the air passage opening 910 arranged there and to enter the second air channel 17b from the air passage opening 910. Of course, in another embodiment, the first temperature conducting portion 930 can also be cylindrical or other shapes.

Optionally, the conducting member 900 is extended toward the interior of the second air channel 17b to form a plurality of second temperature conducting portions 970, and two adjacent second temperature conducting portions 970 form therebetween a second air guide channel 980 to further increase the contact area between the airflow and the conducting member 900, allowing the airflow to be more fully cooled or heated. Specifically, the second temperature conducting portions 970 are in a curved form and are arranged to extend toward the air outlet opening 16. The plurality of second temperature conducting portions 970 are arranged parallel to each other. Of course, in another embodiment, the second temperature conducting portions 970 can also be cylindrical or other shapes.

In another embodiment, optionally, the conducting member 900 may also be only provided with the plurality of first temperature conducting portions 930 or the plurality of second temperature conducting portions 970.

Preferably, the portable temperature regulation device is provided with two conducting members 900, two temperature regulation members 4, two partition members 138, and two heat dissipating members 5 corresponding to each fan 3. Specifically, the air channel 175 is formed between two second separation parts 1384 on the two partition members 138. The two heat dissipating members 5 are connected to each other and are located in the air channel 175. Two first separation parts 1382 on the two partition members 138 are connected to each other to jointly separate the second air channel 173 and the air channel 175. The outside casing 135 is provided with a heat dissipation opening 118 at a location corresponding to the air channel 175. Each second separation part 1384 is provided with an installation opening 1386. The two temperature regulation members 4 are respectively mounted in the two installation openings 1386. One of the conducting members 900 and the outside casing 135 form therebetween a first air channel 17a, and another one of the conducting members 900 and the inside casing 136 form therebetween another first air channel 17a. The outside casing 135 and the inside casing 136 are each provided with an air inlet opening 15 at a location corresponding to the first air channel 17a. The two conducting member 900 jointly form therebetween an air passage channel 17c and a second air channel 17b. The fan 3 is arranged in the air passage channel 17c. The plurality of second temperature conducting portions 970 on the two conducting members 900 are connected to each other and are located in the second air channel 17b. The second air guide channel 980 formed in one of the conducting members 900 communicates with the second air guide channel 980 formed in another one of the conducting members 900. The outside casing 135 and the inside casing 136 are each provided with an air outlet opening 16 at a location corresponding to the second air channel 17b.

What is claimed is:

1. A portable temperature regulation device, which defines a wear space, wherein the portable temperature regulation device comprises a main body, a protrusion portion protruding from the main body, and a temperature conducting member at least partly arranged on the protrusion portion, the main body having a first side and a second side opposite to the first side in a height direction thereof, the protrusion portion being arranged on the first side and protruding in a direction away from the second side.

2. The portable temperature regulation device according to claim 1, wherein the temperature conducting member comprises a first portion arranged on a side surface of the protrusion portion facing the wear space.

3. The portable temperature regulation device according to claim 2, wherein the first portion extends to a side of the protrusion portion away from the main body.

4. The portable temperature regulation device according to claim 3, wherein the temperature conducting member further comprises a second portion arranged on a side surface of the main body facing the wear space, and the first portion and the second portion are separate or integrated together.

5. The portable temperature regulation device according to claim 4, wherein a first air outlet opening is formed in one side of the protrusion portion away from the main body; and
the first portion is arranged to avoid the air outlet opening or the first portion defines a through hole corresponding to the air outlet opening.

6. The portable temperature regulation device according to claim 4, wherein the second portion extends to the second side.

7. The portable temperature regulation device according to claim 4, wherein the main body comprises a base portion and two arm portions arranged at opposite ends of the base portion respectively, the base portion and the two arm portions around the wear space; and
the temperature conducting member further comprises a third portion arranged on a side surface of each of the arm portions facing the wear space, the second portion and the third portions being separate or integrated together.

8. The portable temperature regulation device according to claim 7, wherein each third portion has a height which is substantially equal to that of a corresponding one of the arm portions.

9. The portable temperature regulation device according to claim 1, wherein an end of the protrusion portion away from the main body has a curved structure which comprises an inclined surface inclining from an outer edge away from the wear space to an inner edge close to the wear space.

10. The portable temperature regulation device according to claim 5, wherein the portable temperature regulation device further comprises a temperature regulation member arranged on the main body or the protrusion portion for cooling and/or heating, the temperature conducting member being in thermal conduction contact with the temperature regulation member.

11. The portable temperature regulation device according to claim 10, wherein the portable temperature regulation device further comprises a fan arranged in the main body or the protrusion portion and a heat dissipating member arranged in the main body or the protrusion portion, the heat dissipating member being in thermal conduction contact with the temperature regulation member, a heat dissipation opening being arranged on a side of the main body or the protrusion portion away from the wear space such that airflow generated by the fan is capable of passing through the heat dissipating member and then blowing out from the heat dissipation opening.

12. The portable temperature regulation device according to claim 10, wherein the temperature regulation member is semiconductor cooling sheet sandwiched between the temperature conducting member and the heat dissipating member.

13. The portable temperature regulation device according to claim 11, wherein two said heat dissipation openings are arranged on the side of the main body or the protrusion portion away from the wear space and are spaced apart from each other;
the heat dissipating member comprises two parts each comprising a plurality of fins with gaps formed therebetween; and
each of the two said heat dissipation openings faces the gaps of one corresponding part of the heat dissipating member.

14. The portable temperature regulation device according to claim 13, further comprising another fan accommodated in an interior compartment of the main body; wherein
the main body defines a second air outlet opening at the second side thereof, and a first air inlet opening and a second air inlet opening at an outer side thereof opposite to the wear space;
airflow generated by the fan enters into the interior compartment via the first air inlet opening and exit from the interior compartment via the first air outlet opening and one of the two said heat dissipation openings; and
another airflow generated by said another fan enters into the interior compartment via the second air inlet opening and exit from the interior compartment via the second air outlet opening and another of the two said heat dissipation openings.

15. The portable temperature regulation device according to claim 14, wherein the second air outlet opening comprises two sub air outlet openings arranged side by side in a thickness direction of the main body.

16. The portable temperature regulation device according to claim 14, wherein each of the two said heat dissipation openings has an elongated shape with a length direction and a width direction, the length direction being parallel to the height direction of the main body, the fins being parallel with the width direction or forming an acute angle with respect to the width direction.

17. The portable temperature regulation device according to claim 7, further comprising a battery arranged in an end of one of the arm portions away from the base portion.

18. The portable temperature regulation device according to claim 7, wherein the main body further comprises a flexible connecting member connected between the base portion and each of the arm portions.

19. The portable temperature regulation device according to claim 1, wherein an extension distance of the protrusion portion is in a range of 20 mm-50 mm.

20. The portable temperature regulation device according to claim 1, further comprising a display module mounted to the main body and a covering member covered on the main body and the display module; wherein
a part of the covering member covered on the display module is a light-transmitting region so that when the display module does not emit light, the display module cannot be seen through the light-transmitting region, and when the display module emits light for displaying, the light transmits through the light-transmitting region to achieve an effect of displaying.
